SEQUENCE LISTING (18 sequences)

SEQ ID NO: 1 - DNA, 3088 bp, Homo sapiens
cggagagccgcgcagggcgcgggccgcgcggggtggggcagccggagcgcaggcccccgatccccggcgggcgcccccgggcccccgcgcgcgccccggcctccgggagactggcgcatgccacggagcgcccctcgggccgccgccgctcctgcccgggcccctgctgctgctgctgtcgcctgcgcctgctgccccaactcggcgcccgacttcttcatggtgtgcggaggtcatgttcgctccttagcaggcaaacgacttttctcctcgcctcctcgccccgcatgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggccagtgctacacccgccagttcatcagcagctgcccgtgctggctagaggtcatcttcaacagccggtagccgcgtgcggaggggacagagcgtgagctgagcaggccacacttcaaactactttgctgctaatattttcctcctgagtgcttgcttttcatgcaaactctttggtcgttttttttttgtttgttggttggttttcttcttctcgtcctcgtttgtgttctgttttgtttcgctctttgagaaatagcttatgaaaagaattgttgggggtttttttggaagaaggggcaggtatgatcggcaggacaccctgataggaagaggggaagcagaaatccaagcaccaccaaacacagtgtatgaaggggggcggtcatcatttcacttgtcaggagtgtgtgtgagtgtgagtgtgcggctgtgtgtgcacgcgtgtgcaggagcggcagatggggagacaacgtgctctttgttttgtgtctcttatggatgtccccagcagagaggtttgcagtcccaagcggtgtctctcctgccccttggacacgctcagtggggcagaggcagtacctgggcaagctggcggctggggtcccagcagctgccaggagcacggctctgtccccagcctgggaaagcccctgcccctcctctccctcatcaaggacacgggcctgtccacaggcttctgagcagcgagcctgctagtggccgaaccagaaccaattattttcatccttgtcttattcccttcctgccagcccctgccattgtagcgtctttcttttttggccatctgctcctggatctccctgagatgggcttcccaagggctgccggggcagccccctcacagtattgctcacccagtgccctctcccctcagcctctcccctgcctgccctggtgacatcaggtttttcccggacttagaaaaccagctcagcactgcctgctcccatcctgtgtgttaagctctgctattaggccagcaagcggggatgtccctgggagggacatgcttagcagtccccttccctccaagaaggatttggtccgtcataacccaaggtaccatcctaggctgacacctaactcttctttcatttcttctacaactcatacactcgtatgatacttcgacactgttcttagctcaatgagcatgtttagactttaacataagctatttttctaactacaaaggtttaaatgaacaagagaagcattctcattggaaatttagcattgtagtgctttgagagagaaaggactcctgaaaaaaaacctgagatttattaaagaaaaaaatgtattttatgttatatataaatatattattacttgtaaatataaagacgttttataagcatcattatttatgtattgtgcaatgtgtataaacaagaaaaataaagaaaagatgcactttgctttaatataaatgcaaataacaaatgccaaattaaaaaagataaacacaagattggtgtttttttctatgggtgttatcacctagctgaatgtttttctaaaggagtttatgttccattaaacgatttttaaaatgtacacttgaa

SEQ ID NO: 2 - DNA, 21 bp, Artificial Sequence
  Description of Artificial Sequence Synthetic oligonucleotide
gtcgccccttctccccgcagc

SEQ ID NO: 3 - DNA, 20 bp, Artificial Sequence
  Description of Artificial Sequence Synthetic oligonucleotide
  modified_base (3)..(3): 5-methyl-2′-deoxycytidine
  modified_base (16)..(16): 5-methyl-2′-deoxycytidine
gtcgccccttctccccgcag

SEQ ID NO: 4 - DNA, 21 bp, Artificial Sequence
  Description of Artificial Sequence Synthetic oligonucleotide
  modified_base (3)..(3): 5-methyl-2′-deoxycytidine
  modified_base (16)..(16): 5-methyl-2′-deoxycytidine
gtcgccccttctccccgcagc

SEQ ID NO: 5 - DNA, 20 bp, Artificial Sequence
  Description of Artificial Sequence Synthetic oligonucleotide
  modified_base (3)..(3): Nitrogenous base selected from the group consisting of cytosine, 5-methylcytosine or 2′-O-methylcytosine
  modified_base (4)..(4): Nitrogenous base selected from the group consisting of guanine, 5-methylguanine or 2′-O-methylguanine
  modified_base (16)..(16): Nitrogenous base selected from the group consisting of cytosine, 5-methylcytosine or 2′-O-methylcytosine
  modified_base (17)..(17): Nitrogenous base selected from the group consisting of guanine, 5-methylguanine or 2′-O-methylguanine
  See specification as filed for detailed description of substitutions and preferred embodiments
gtcgccccttctccccgcag

SEQ ID NO: 6 - DNA, 1522 bp, Homo sapiens
acaccatgtttggtaaataagtgttttggtgttgtgcaagggtctggtttcagcctgaagccatctcagagctgtctgggtctctggagactggagggacaacctagtctagagcccatttgcatgagaccaaggatcctcctgcaagagacaccatcctgagggaagagggcttctgaaccagcttgacccaataagaaattcttgggtgccgacgcggaagcagattcagagcctagagccgtgcctgcgtccgtagtttccttctagcttcttttgatttcaaatcaagacttacagggagagggagcgataaacacaaactctgcaagatgccacaaggtcctcctttgacatccccaacaaagaggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaa

SEQ ID NO: 7 - DNA, 2471 bp, Homo sapiens
ccccaataaatataggactggagatgtctgaggctcattctgccctcgagcccaccgggaacgaaagagaagctctatctcccctccaggagcccagctatgaactccttctccacaagtaagtgcaggaaatccttagccctggaactgccagcggcggtcgagccctgtgtgagggaggggtgtgtggcccagggagggctggcgggcggccagcagcagaggcaggctcccagctgtgctgtcagctcacccctgcgctcgctcccctccggcacaggcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggtgggtgtgtcctcattccctcaacttggtgtgggggaagacaggctcaaagacagtgtcctggacaactcagggatgcaatgccacttccaaaagagaaggctacacgtaaacaaaagagtctgagaaatagtttctgattgttattgttaaatctttttttgtttgtttggttggttggctctcttctgcaaaggacatcaataactgtattttaaactatatattaactgaggtggattttaacatcaatttttaatagtgcaagagatttaaaaccaaaggcgggggggcgggcagaaaaaagtgcatccaactccagccagtgatccacagaaacaaagaccaaggagcacaaaatgattttaagattttagtcattgccaagtgacattcttctcactgtggttgtttcaattctttttcctaccttttaccagagagttagttcagagaaatggtcagagactcaagggtggaaagaggtaccaaaggctttggccaccagtagctggctattcagacagcagggagtagacttgctggctagcatgtggaggagccaaagctcaataagaaggggcctagaatgaaacccttggtgctgatcctgcctctgccatttctacttaagccagggtttctcatatgttaacatgcatgggaattccctgggcatcttcttgtggtgtggagtctgacttagcaagcctcgggtgggtttgagggtcaaatttctaccaggcttatatccctggtgatgctgcagaattccaggaccacacttggaggtttaaggccttccacaagttacttatcccatatggtgggtctatggaaaggtgtttcccagtcctctttacaccaccggatcagtggtctttcaacagatcctaaagggatggtgagagggaaactggagaaaagtatcagatttagaggccactgaagaacccatattaaaatgcctttaagtatgggctcttcattcatatactaaatatgaactatgtgccaggcattatttcatatgacagaatacaaacaaataagatagtgatgctggtcaggcttggtggctcatgcctgtattccctaaactttgggagcctaaggtgagaactccttgaactcctaaggccaggagttcaagaccagcctggataacatagcaagaccccatctctacaaaaaaccaaaaccaaacaaacaaaaatgatagtggtgcttccctcaggatgcttgtggtctaatgggagacagaacagcaaagggatgattagaagttggttgctgtgagccaggcacagtgctgatataatcccagcgctatgggaggctgaggtgggtggatcatttgaggccaggagtttaagaccagcctggtcaacatggtaaaaccccatctctacttaaaaatacaaaaaagttagccaggcatggtggcatacacctgtaacccagctactcaggaggctgaggcacatgaatcacttgaacccaggaggcagaggttgctgtgcaccactgcactccagcctgggtgacagaacgagaccttgactcaaaaaaaaaaaaaagaagtttgttgctatggaagggtcctactcagagcaggcaccccagttaatctcattcaccccacatttcacatttgaacatcatcccatagcccagagcatccctccactgcaaaggatttattcaacatttaaacaatcctttttactttcattttc

SEQ ID NO: 8 - DNA, 1686 bp, Homo sapiens
cagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaaaaaaaaaaaa

SEQ ID NO: 9 - DNA, 20 bp, Artificial Sequence
  Description of Artificial Sequence Synthetic primer
ccactcacctcttcagaacg

SEQ ID NO: 10 - DNA, 21 bp, Artificial Sequence
  Description of Artificial Sequence Synthetic primer
gcctctttgctgctttcacac

SEQ ID NO: 11 - DNA, 20 bp, Artificial Sequence
  Description of Artificial Sequence Synthetic primer
aggcggtgcttgttcctcag

SEQ ID NO: 12 - DNA, 20 bp, Artificial Sequence
  Description of Artificial Sequence Synthetic primer
ggctacaggcttgtcactcg

SEQ ID NO: 13 - DNA, 27 bp, Artificial Sequence
  Description of Artificial Sequence Synthetic primer
aagatgacccagatcatgtttgagacc

SEQ ID NO: 14 - DNA, 20 bp, Artificial Sequence
  Description of Artificial Sequence Synthetic primer
agccagtccagacgcaggat

SEQ ID NO: 15 - DNA, 20 bp, Artificial Sequence
  Description of Artificial Sequence Synthetic primer
cggatctcaggcattcctcg

SEQ ID NO: 16 - DNA, 20 bp, Artificial Sequence
  Description of Artificial Sequence Synthetic primer
ggcacagcatctggacagtc

SEQ ID NO: 17 - DNA, 27 bp, Artificial Sequence
  Description of Artificial Sequence Synthetic primer
aagatgacccagatcatgtttgagacc

SEQ ID NO: 18 - DNA, 20 bp, Artificial Sequence
  Description of Artificial Sequence Synthetic primer
agccagtccagacgcaggat

(19) United States
(12) Patent Application Publication
(10) Pub. No.: US 2021/0207143 A1
(43) Pub. Date: Jul. 8, 2021

(54) Methods of Treating Celiac Disease Using SMAD7 Inhibition

(71) Applicant: Nogra Pharma Limited, Dublin 2 (IE)

(72) Inventor: Giovanni Monteleone, Grottaferrata (IT)

(73) Assignees: Nogra Pharma Limited, Dublin 2 (IE); Nogra Pharma Limited, Dublin 2 (IE)

(21) Appl. No.: 16/079,232

(22) PCT Filed: Feb. 24, 2017
(86) PCT No.: PCT/US07/54380

§ 371 Date: Aug. 23, 2018

(51) Int. Cl. C12N 15/113; A61K 9/00; A61K 47/34; A61P 37/06

(52) U.S. Cl. CPC C12N 15/1136; A61K 9/0053; C12N 2310/315; A61P 37/06; C12N 2310/11; A61K 47/34

(57) ABSTRACT

The present invention relates to methods of treating, preventing, and/or managing celiac disease by inhibiting SMAD7. The invention is also directed to methods of monitoring effectiveness of treatment or management of celiac disease using a SMAD7 antisense oligonucleotide, as well as methods of regulating SMAD7 antisense oligonucleotide treatment, based on analysis of Transforming Growth Factor-β (TGF-β) signaling activity.

### CROSS REFERENCE TO RELATED APPLICATION

This application claims the benefit of U.S. Provisional Application No. 62/299,543, filed Feb. 24, 2016, and U.S. Provisional Application No. 62/382,461, filed Sep. 1, 2016, the entire contents of each of which are herein incorporated by reference for all purposes.

### FIELD OF THE INVENTION

The present invention relates to methods of treating celiac disease by inhibiting Mothers Against Decapentaplegic Homolog 7 (SMAD7), Tumor Necrosis Factor α (TNFα), and/or Interleukin-6 (IL-6). The invention is also directed to methods of monitoring effectiveness of treatment or management of celiac disease using a SMAD7 antisense oligonucleotide, as well as methods of regulating SMAD7 antisense oligonucleotide treatment, based on analysis of Transforming Growth Factor-β (TGF-β) signaling activity.

### SUMMARY

In one aspect, the invention relates to a method of treating, managing, or preventing celiac disease or enhancing TGF-β signaling in a cell of a patient with celiac disease, by inhibiting one or more of SMAD7, IL-6, and TNFα in a patient suffering from celiac disease. In another aspect the invention relates to a method of treating, managing, or preventing celiac disease or enhancing TGF-β signaling in a cell of a patient with celiac disease, by inhibiting one or more of SMAD7, IL-6, and TNFα in a cell, for example, an intestinal cell. In yet another aspect, the invention relates to a method of treating or managing celiac disease in a patient with celiac disease, by administering to the patient an effective amount of one or more of a specific inhibitor of SMAD7, a specific inhibitor of IL-6, or a specific inhibitor of TNFα. In a further aspect, the invention provides a specific inhibitor of SMAD7, a specific inhibitor of IL-6, or a specific inhibitor of TNFα for use as a medicament. The invention also relates to a specific inhibitor of SMAD7, a specific inhibitor of IL-6, or a specific inhibitor of TNFα for use in treating, preventing, or managing celiac disease, preferably wherein the treatment is via a method as described herein. Use of a specific inhibitor of SMAD7, a specific inhibitor of IL-6, or a specific inhibitor of TNFα in the manufacture of a medicament for the treatment, prevention, or management of celiac disease by a method described herein is also provided herein.

In some embodiments, the specific inhibitor of SMAD7 is a SMAD7 antisense oligonucleotide. In some embodiments, the specific inhibitor of IL-6 is an IL-6 antisense oligonucleotide. In some embodiments, the specific inhibitor of TNFα is a TNFα antisense oligonucleotide.

Embodiments of the invention include methods of enhancing TGF-β signaling in a cell of a patient with celiac disease and/or inhibiting SMAD7, TNFα and/or IL-6 in such a cell. In some embodiments of the invention, the cell is an intestinal cell, for example, a small intestinal cell, a large intestinal cell, or a lamina propria mononuclear cell.

Embodiments of the invention include methods of treating and/or preventing celiac disease in a patient. In some embodiments the patient is not suffering from an inflammatory bowel disease. In some embodiments, the patient is suffering from an inflammatory bowel disease. In some embodiments, the patient has celiac disease, which is preceded by inflammatory bowel disease. In some embodiments, the inflammatory bowel disease is Crohn's disease or ulcerative colitis. In some embodiments, the celiac disease being treated, managed, or prevented is refractory celiac disease.

In some embodiments of the invention the patient is a mammal, for example, a primate, for example, a human.

The invention relates in part to the fact that levels of a TGF-β signaling activity in a patient with celiac disease correlate with celiac disease state and can be used as a means for monitoring disease state and managing responsiveness to celiac disease treatment with an anti-SMAD7 therapy, an anti-IL-6 therapy, and/or an anti-TNFα therapy. One can use monitoring and analysis of TGF-β signaling activity in a patient with celiac disease to determine appropriate levels of SMAD7 antisense oligonucleotide administration and to regulate and adjust SMAD7 antisense oligonucleotide treatment. Similarly, one can use monitoring and analysis of TGF-β signaling activity in a patient with celiac disease to determine appropriate levels of specific inhibitor of IL-6 or TNFα administration and to regulate and adjust specific inhibitor of IL-6 or TNFα treatment.

Assessment of TGF-β signaling activity may include measuring levels of a TGF-β signaling analyte, for example, but not limited to, a level of Transforming Growth Factor-β1 (TGF-β1), Transforming Growth Factor-β2 (TGF-β2), Transforming Growth Factor-β3 (TGF-β3), Mothers Against Decapentaplegic Homolog 2 (SMAD2), Mothers Against Decapentaplegic Homolog 3 (SMAD3), Mothers Against Decapentaplegic Homolog 4 (SMAD4), phosphorylated SMAD2 (p-SMAD2), or phosphorylated SMAD3 (p-SMAD3). Assessment of TGF-β signaling activity may include detecting post-transcriptional modifications, for example, phosphorylation or ubiquitination, of proteins of interest, for example, SMAD3. Assessment of TGF-β signaling activity may include detecting an activity associated with enhancing or promoting TGF-β signaling such as binding of gene promoter regions associated with downstream TGF-β signaling or binding of TGF-β receptors by an activating ligand.

It will be appreciated that it is advantageous to be able to determine shortly after commencing treatment, shortly before stopping treatment, or shortly after stopping treatment, whether a celiac disease patient is responsive to treatment with an anti-IL-6 therapy, an anti-TNFα therapy, or an anti-SMAD7 therapy, in particular, a SMAD7 antisense oligonucleotide. Modulation of TGF-β signaling activity levels in a patient with celiac disease, as described herein, are useful for evaluating the efficacy of and responsiveness to treatment with an anti-IL-6 therapy, an anti-TNFα therapy, or an anti-SMAD7 therapy in a subject having celiac disease. Furthermore, it will be appreciated that it is advantageous to be able to evaluate and modulate administration of an anti-IL-6 therapy, an anti-TNFα therapy, or an anti-SMAD7 therapy in a patient with celiac disease based on levels, or changes in levels, of a biomarker, e.g., p-SMAD3 or TGF-β, or other TGF-β signaling activities that correlate with disease state. Thus, the invention provides methods for analyzing levels of TGF-β signaling activity in a patient being treated with or who has been administered an anti-IL-6 therapy, an anti-TNFα therapy, or an anti-SMAD7 therapy, e.g., a SMAD7 antisense oligonucleotide, and adjusting dosage levels based on TGF-β signaling activity or changes in TGF-β signaling activity determined by an analyzing step, following a dose of the anti-IL-6 therapy, the anti-TNFα therapy, or the anti-SMAD7 therapy. Advantageously, the methods of the invention will ultimately assist physicians in choosing effective therapies and monitoring and adjusting treatment with said therapies. Furthermore, methods of the invention may lead to improvements in celiac disease treatment efficacy for patients, with reduction in overall patient costs.

In a first aspect, the invention provides methods for treating or managing celiac disease in a patient having celiac disease. In one embodiment, the method includes the following steps: (a) administering to the patient an initial dose of a specific inhibitor of IL-6, a specific inhibitor of TNFα, or a SMAD7 antisense oligonucleotide; (b) analyzing the level of a TGF-β signaling activity in the patient; and (c) if the level of TGF-β signaling activity is below normal levels of TGF-β signaling activity, then administering to the patient a subsequent dose that is greater than or equal to the initial dose. Alternatively, if in step (c), the level of TGF-β signaling activity is above normal levels of TGF-β signaling activity as determined in step (b), then step (c) includes administering to the patient a subsequent dose that is equal to or smaller than the initial dose.

In some embodiments, the invention relates to a specific inhibitor of SMAD7, a specific inhibitor of IL-6, or a specific inhibitor of TNFα for use as a medicament. The invention also relates to a specific inhibitor of SMAD7, a specific inhibitor of IL-6, or a specific inhibitor of TNFα for use in treating celiac disease, preferably wherein treatment is by a method as described herein. Use of a specific inhibitor of SMAD7, a specific inhibitor of IL-6, or a specific inhibitor of TNFα in the manufacture of a medicament for the treatment of celiac disease is also described herein. In further embodiments, the invention may include a specific inhibitor of IL-6, a specific inhibitor of TNFα, or a SMAD7 antisense oligonucleotide for use in a method of treating or managing celiac disease. For instance, in some embodiments, the invention includes a specific inhibitor of IL-6, a specific inhibitor of TNFα, or a SMAD7 antisense oligonucleotide for use in a method for treating or managing celiac disease in a patient having celiac disease, wherein the method comprises analyzing the level of a TGF-β signaling activity in the patient to determine appropriate levels of SMAD7 antisense oligonucleotide administration. In some embodiments, the invention includes a specific inhibitor of IL-6, a specific inhibitor of TNFα, or a SMAD7 antisense oligonucleotide for this use, wherein the method comprises the steps of: (a) administering to the patient an initial dose of the specific inhibitor of IL-6, the specific inhibitor of TNFα, or the SMAD7 antisense oligonucleotide; (b) analyzing the level of TGF-β signaling activity in the patient; and (c) if the level of TGF-β signaling activity is below normal levels of TGF-β signaling activity, then administering to the patient a subsequent dose of the specific inhibitor of IL-6, the specific inhibitor of TNFα, or the SMAD7 antisense oligonucleotide that is greater than or equal to the initial dose, or, if the level of TGF-β signaling activity is above normal levels of TGF-β signaling activity, then administering to the patient a subsequent dose of the specific inhibitor of IL-6, the specific inhibitor of TNFα, or the SMAD7 antisense oligonucleotide that is equal to or smaller than the initial dose.

In another aspect of the invention, the invention provides methods for treating or managing celiac disease in a patient having celiac disease with respect to administration of an initial dose of a specific inhibitor of IL-6, a specific inhibitor of TNFα, or a SMAD7 antisense oligonucleotide. In one embodiment, the invention provides a method for treating or managing celiac disease in a patient having celiac disease, where the method includes the following steps: (a) analyzing the level of a TGF-β signaling activity in the patient; and (b) if the level of TGF-β signaling activity is below normal levels of TGF-β signaling activity, then administering to the patient an initial dose of a specific inhibitor of IL-6, a specific inhibitor of TNFα, or a SMAD7 antisense oligonucleotide. In a particular embodiment, the invention provides a method for treating or managing celiac disease in a patient having celiac disease, where the method includes the following steps: (a) analyzing the level of a TGF-β signaling activity in the patient; and (b) if the level of TGF-β signaling activity is below 0.01 pg/ml, 0.1 pg/ml, 1 pg/ml 2 pg/ml, 3 pg/ml, 4 pg/ml, 5 pg/ml, 6 pg/ml, 7 pg/ml, 8 pg/ml, 9 pg/ml, 10 pg/ml, 11 pg/ml, 12 pg/ml, 13 pg/ml, 14 pg/ml, 15 pg/ml, 17.5 pg/ml, 20 pg/ml, 22.5 pg/ml, 25 pg/ml, 30 pg/ml, or 35 pg/ml, then administering to the patient an initial dose of a specific inhibitor of IL-6, a specific inhibitor of TNFα, or a SMAD7 antisense oligonucleotide.

Additionally, the method may further include the steps of: (c) analyzing the level of a TGF-β signaling activity in the patient after said administering step, i.e., step (b); and (d) if the level of TGF-β signaling activity is below normal levels of TGF-β signaling activity, then administering to the patient a subsequent dose that is greater than or equal to the initial dose. Alternatively, if in step (d), the level of TGF-β signaling activity is above normal levels of TGF-β signaling activity, as determined in step (c), then step (d) includes administering to the patient a subsequent dose that is equal to or smaller than the initial dose. In some instances, if the subsequent dose administered in step (d) is equal to or greater than the maximum tolerated dose (MTD), then the method includes the step of terminating the treatment.

In some embodiments, the invention comprises methods of treating or managing celiac disease, dependent upon establishment of a control level of a TGF-β signaling activity. For example, in a particular embodiment, the method for treating or managing celiac disease in a patient having celiac disease includes the steps of (a) establishing a control level of a TGF-β signaling activity for the patient; (b) administering to the patient an initial dose of a specific inhibitor of IL-6, a specific inhibitor of TNFα, or a SMAD7 antisense oligonucleotide; (c) analyzing the level of TGF-β signaling activity in the patient; and (d) if the level of TGF-β signaling activity is higher than the control level, then administering to the patient a subsequent dose that is the same as the initial dose or smaller than the initial dose or terminating the treatment. Alternatively, if the level of TGF-β signaling activity determined in step (c) is unchanged or decreased compared to the control level, then the method includes a step (d) of administering to the patient a subsequent dose that is the same as the initial dose or greater than the initial dose.

In some embodiments, the method comprises the steps of (a) analyzing a first level of a TGF-β signaling activity in the patient; (b) administering to the patient an initial dose of a specific inhibitor of IL-6, a specific inhibitor of TNFα, or a SMAD7 antisense oligonucleotide (AON); and (c) analyzing a second level of TGF-β signaling activity in the patient after the administering step. In an embodiment of the invention, if the second level of TGF-β signaling activity is the same or lower than the first level of TGF-β signaling activity, then: administering to the patient a subsequent dose that is equal to or greater than the initial dose, and/or administering to the patient a subsequent dose at an equal or higher frequency than the initial dose. Alternatively, if the second level of TGF-β signaling activity is higher than the first level of TGF-β signaling activity, then administering to the patient a subsequent dose that is equal to or smaller than the initial dose, and/or administering to the patient a subsequent dose at an equal or lower frequency than the initial dose.

In some embodiments of the invention, the second level of TGF-β signaling activity is higher than the first level of TGF-β signaling activity. For example, in some embodiments, the second level of TGF-β signaling activity is about 10% higher, about 20% higher, about 30% higher, about 40% higher, about 50% higher, about 60% higher, about 70% higher, about 80% higher, about 90% higher, about 100% higher, or more than the first level of TGF-β signaling activity. In some embodiments, the second level of TGF-β signaling activity is about 10% to about 20% higher, about 20% to about 30% higher, about 30% to about 40% higher, about 40% to about 50% higher, about 50% to about 60% higher, about 60% to about 70% higher, about 70% to about 80% higher, about 80% to about 90% higher, or about 90% to about 100% higher than the first level of TGF-β signaling activity. Alternatively, in some embodiments, the second level of TGF-β signaling activity is lower than the first level of TGF-β signaling activity. For example, in some embodiments, the second level of TGF-β signaling activity is about 10% lower, about 20% lower, about 30% lower, about 40% lower, about 50% lower, about 60% lower, about 70% lower, about 80% lower, about 90% lower, or about 100% lower than the first level of TGF-β signaling activity. In some embodiments, the second level of TGF-β signaling activity is about 10% to about 20% lower, about 20% to about 30% lower, about 30% to about 40% lower, about 40% to about 50% lower, about 50% to about 60% lower, about 60% to about 70% lower, about 70% to about 80% lower, about 80% to about 90% lower, or about 90% to about 100% lower than the first level of TGF-β signaling activity.

In some embodiments, the invention comprises a method for treating or managing celiac disease in a patient having celiac disease, wherein the method includes the steps of (a) administering to the patient an initial dose of a specific inhibitor of IL-6, a specific inhibitor of TNFα, or a SMAD7 antisense oligonucleotide; and (b) analyzing the level of a TGF-β signaling activity in the patient after the administering step. In some embodiments, if the level of TGF-β signaling activity is below normal levels of TGF-β signaling activity, then the patient is administered a subsequent dose that is greater than or equal to the initial dose, and/or administering to the patient a subsequent dose at an equal or higher frequency than the initial dose. In some embodiments, if the level of a TGF-β signaling activity is above normal levels of TGF-β signaling activity, then the patient is administered a subsequent dose that is equal to or smaller than the initial dose and/or administering to the patient a subsequent dose at an equal or lower frequency than the initial dose.

In some embodiments of the invention, the level of a TGF-β signaling activity is higher than the normal level of TGF-β signaling activity. For example, in some embodiments, the level of TGF-β signaling activity is about 10% higher, about 20% higher, about 30% higher, about 40% higher, about 50% higher, about 60% higher, about 70% higher, about 80% higher, about 90% higher, about 100% higher, or more than the normal level of TGF-β signaling activity. In some embodiments, the level of TGF-β signaling activity is about 10% to about 20% higher, about 20% to about 30% higher, about 30% to about 40% higher, about 40% to about 50% higher, about 50% to about 60% higher, about 60% to about 70% higher, about 70% to about 80% higher, about 80% to about 90% higher, or about 90% to about 100% higher than the normal level of TGF-β signaling activity. In some embodiments, the level of TGF-β signaling activity is lower than the normal level of TGF-β signaling activity. For example, in some embodiments, the level of TGF-β signaling activity is about 10% lower, about 20% lower, about 30% lower, about 40% lower, about 50% lower, about 60% lower, about 70% lower, about 80% lower, about 90% lower, or about 100% lower than the normal level of TGF-β signaling activity. In some embodiments, the second level of TGF-β signaling activity is about 10% to about 20% lower, about 20% to about 30% lower, about 30% to about 40% lower, about 40% to about 50% lower, about 50% to about 60% lower, about 60% to about 70% lower, about 70% to about 80% lower, about 80% to about 90% lower, or about 90% to about 100% lower than the normal level of TGF-β signaling activity.

In some embodiments, the invention includes a specific inhibitor of IL-6, a specific inhibitor of TNFα, or a SMAD7 antisense oligonucleotide for use in a method for treating or managing celiac disease in a patient having celiac disease, wherein the method includes (a) analyzing the level of a TGF-β signaling activity in the patient; and (b) if the level of TGF-β signaling activity is below normal levels of TGF-β signaling activity, then administering to the patient an initial dose of the specific inhibitor of IL-6, the specific inhibitor of TNFα, or the SMAD7 antisense oligonucleotide.

The level of TGF-β signaling activity may be analyzed at varying time points following an administering step (b). For instance, in some embodiments, following an administering step (b), the level of TGF-β signaling activity is analyzed at least 1 day, at least 3 days, at least 5 days, at least 1 week, at least 2 weeks, at least 3 weeks, at least 1 month, at least 2 months, at least 4 months, or at least 6 months after said administration step. In some embodiments, the level of TGF-β signaling activity is analyzed immediately after said administration step. In yet other embodiments, the level of TGF-β signaling activity is analyzed about 7 days, about 10 days, about 15 days, about 20 days, about 25 days, or about 28 days after said administration step.

Normal levels or a control level of a TGF-β signaling activity may be determined based on numerical reference values or with respect to levels of TGF-β signaling activity in a healthy control group. For instance, in some embodiments, a control level or normal levels of TGF-β signaling activity is analyzed by measuring the concentration of a TGF-β signaling analyte, for example, by measuring the concentration of TGF-β1, TGF-β2, TGF-β3, SMAD2, SMAD3, SMAD4, p-SMAD2, or p-SMAD3. Concentrations of TGF-β, SMAD2, SMAD3, p-SMAD2, or p-SMAD3 used to determine a control level or normal levels of TGF-β signaling activity may be about 0.01 pg/ml, about 0.1 pg/ml, about 1 pg/ml, about 2 pg/ml, about 3 pg/ml, about 4 pg/ml, about 5 pg/ml, about 6 pg/ml, about 7 pg/ml, about 8 pg/ml, about 9 pg/ml, about 10 pg/ml, about 11 pg/ml, about 12 pg/ml, about 13 pg/ml, about 14 pg/ml, about 15 pg/ml, about 16 pg/ml, about 17 pg/ml, about 17.5 pg/ml, about 18 pg/ml, about 19 pg/ml, about 20 pg/ml, about 22.5 pg/ml, about 25 pg/ml, about 30 pg/ml, or about 35 pg/ml. In other embodiments of the invention, a control level or normal levels of TGF-β signaling activity are defined as median levels of TGF-β signaling activity in a healthy control group. A healthy control group may be defined based on various criteria related to genetic background, habits, and physical attributes matched to the same set of criteria in the patient. For instance, in some embodiments, the healthy control group and the patient having celiac disease are matched with respect to age, gender, ethnic origin, smoking habits, dietary habits, body-mass index (BMI), recreational drug use, medical drug use, drug use related to celiac disease, and/or exercise habits. Other factors that can be matched between the patient and control group include, but are not limited to, clinical criteria (e.g., severity of celiac disease-related symptoms), metabolism, celiac disease patient's personal disease history, genetic factors, celiac disease patient's family disease history, exposure to environmental factors (e.g., pollutants, toxins, allergens), and life-style (e.g., urban, suburban, or rural place of work and/or domicile).

In various embodiments of the invention, the initial dose of a specific inhibitor of IL-6, a specific inhibitor of TNFα, or a SMAD7 antisense oligonucleotide administered to a patient having celiac disease may vary. For instance, in some embodiments, the initial dose of a specific inhibitor of IL-6, a specific inhibitor of TNFα, or a SMAD7 antisense oligonucleotide administered to a patient having celiac disease is less than 500 mg/day, less than 400 mg/day, less than 300 mg/day, less than 200 mg/day, less than 100 mg/day, less than 90 mg/day, less than 80 mg/day, less than 70 mg/day, less than 60 mg/day, less than 50 mg/day, less than 40 mg/day, less than 30 mg/day, less than 20 mg/day, or less than 10 mg/day. Alternatively, in other embodiments, the initial dose is at least 1 mg/day, at least 5 mg/day, at least 10 mg/day, at least 20 mg/day, at least 30 mg/day, at least 40 mg/day, at least 50 mg/day, at least 60 mg/day, at least 70 mg/day, at least 80 mg/day, at least 90 mg/day, at least 100 mg/day, at least 200 mg/day, at least 300 mg/day, at least 400 mg/day, or at least 500 mg/day. In yet other embodiments, the initial dose is about 5 mg/day, about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 200 mg/day, about 300 mg/day, about 400 mg/day, or about 500 mg/day. In some embodiments, the initial dose is 5 mg/day, 10 mg/day, 20 mg/day, 30 mg/day, 40 mg/day, 50 mg/day, 60 mg/day, 70 mg/day, 80 mg/day, 90 mg/day, 100 mg/day, 110 mg/day, 120 mg/day, 130 mg/day, 140 mg/day, 150 mg/day, 160 mg/day, 170 mg/day, 180 mg/day, 190 mg/day, or 200 mg/day.

In some embodiments of the invention, after analyzing the level of a TGF-β signaling activity in the patient in a step (b) or (c), if the level of TGF-β signaling activity is below normal levels of TGF-β signaling activity, then the method may include the step of administering to the patient a subsequent dose that is greater than the initial dose. In some embodiments, after analyzing the level of TGF-β signaling activity in the patient in a step (b) or (c), if the level of TGF-β signaling activity is above normal levels of TGF-β signaling activity, then the method may include the step of administering to the patient a subsequent dose that is smaller than the initial dose.

The invention also provides a method for determining the level of a subsequent dose of a specific inhibitor of IL-6, a specific inhibitor of TNFα, or a SMAD7 antisense oligonucleotide with respect to an initial dose of, respectively, a specific inhibitor of IL-6, a specific inhibitor of TNFα, or a SMAD7 antisense oligonucleotide based on levels of TGF-β signaling activity in a patient having celiac disease. For instance, in embodiments of the invention described herein, if TGF-β signaling activity levels in a patient having celiac disease are below normal levels or a control level following an initial administration step (a) or (b), the subsequent dose administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, at least about 160 mg/day, at least about 170 mg/day, at least about 180 mg/day, at least about 190 mg/day, or at least about 200 mg/day greater than the initial dose.

Alternatively, in some embodiments, if TGF-β signaling activity levels in a patient having celiac disease are above a control level or normal levels following an initial administration step (a) or (b), the subsequent dose administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, or at least about 100 mg/day smaller than the initial dose. Furthermore, in some embodiments, the initial dose administered in an initial administration step (a) or (b) is between about 10 mg/day and 100 mg/day, about 5 mg/day and 200 mg/day, about 10 mg/day and 50 mg/day, about 50 mg/day and 100 mg/day, and about 100 mg/day and about 200 mg/day, and the subsequent dose administered in a step (c) or (d) is between about 30 mg/day and 200 mg/day, about 5 mg/day and 30 mg/day, about 20 mg/day and 50 mg/day, about 50 mg/day and 100 mg/day, or about 100 mg/day and 200 mg/day.

The invention also provides methods for modulating treatment with a specific inhibitor of IL-6, a specific inhibitor of TNFα, or a SMAD7 antisense oligonucleotide in a patient with celiac disease based on a comparison of relative levels TGF-β signaling activity in a patient before and after an initial administering step. The method includes the following steps: (a) analyzing the level of TGF-β signaling activity in the patient; and (b) if the level of TGF-β signaling activity is below normal levels of TGF-β signaling activity, then administering to the patient an initial dose of a SMAD7 antisense oligonucleotide; (c) analyzing the level of TGF-β signaling activity in the patient after said administering step; and (d) if the level of TGF-β signaling activity is higher or increased after said administration step than the level of TGF-β signaling activity before said administration step, then administering to the patient a subsequent dose that is the same as the initial dose or smaller than the initial dose. Alternatively, in step (d) if the level of TGF-β signaling activity is unchanged or decreased after said administration step (i.e., step (b)) compared to the level of TGF-β signaling activity before said administration step, then step (d) includes administering to the patient a subsequent dose that is greater than the initial dose or terminating the treatment.

According to methods of the invention, a change in TGF-β signaling activity levels observed after an initial administration step (of a specific inhibitor of IL-6, a specific inhibitor of TNFα, or a SMAD7 antisense oligonucleotide) compared to TGF-β signaling activity levels prior to the administration step can be compared, for example, as a change in percent of TGF-β signaling activity levels, to determine the amount of a subsequent dose of the specific inhibitor of IL-6, the specific inhibitor of TNFα, or the SMAD7 antisense oligonucleotide to be administered to a patient with celiac disease. For example, in some embodiments, if the level of TGF-β signaling activity is at least 10%, at least 20%, at least 30%, at least 40%, at least 50%, at least 60%, at least 70%, at least 80%, at least 90%, or at least 95% increased after said administration step (e.g., an administration step (b)) compared to the level of TGF-β signaling activity before said administration step, then the method includes a step (e.g., an administration step (d)) of administering to the patient a subsequent dose that is the same as the initial dose or smaller than the initial dose.

The invention also relates to the discovery that a change in TGF-β signaling activity levels relative to a TGF-β signaling activity levels before treatment may be used as an indication that the patient is likely to experience full or partial clinical amelioration of celiac disease. An increase in levels of TGF-β signaling activity may also be used to determine the length of time during which the patient is likely to experience clinical amelioration. For example, in some embodiments, the method includes determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of celiac disease for a time period of at least 1 week, at least 2 weeks, at least 3 weeks, at least 4 weeks, at least 6 weeks or at least 8 weeks, if the level of TGF-β signaling activity after said administering step is increased at least 10%, at least 20%, at least 30%, at least 40%, at least 50%, at least 60%, or at least 70% compared to the level of TGF-β signaling activity before the administration step.

In some embodiments, the invention provides a method of treating or managing celiac disease in a patient with below normal levels of TGF-β signaling activity, where the method includes administering to the patient a dose of a specific inhibitor of IL-6, a specific inhibitor of TNFα, or a SMAD7 antisense oligonucleotide. Furthermore, in some embodiments, the invention provides methods for treating or managing celiac disease in a patient who has below normal TGF-β signaling activity levels following administration of a dose of a specific inhibitor of IL-6, a specific inhibitor of TNFα, or a SMAD7 antisense oligonucleotide, where the patient is administered a further dose of the specific inhibitor of IL-6, the specific inhibitor of TNFα, or the SMAD7 antisense oligonucleotide that is greater than or equal to the prior dose. Similarly, in some embodiments, the invention provides methods for treating or managing celiac disease in a patient having celiac disease who has above normal TGF-β signaling activity levels following administration of a dose of a specific inhibitor of IL-6, a specific inhibitor of TNFα, or a SMAD7 antisense oligonucleotide. In the latter case, the method will include administering to the patient a further dose of the specific inhibitor of IL-6, the specific inhibitor of TNFα, or the SMAD7 antisense oligonucleotide that is less than or equal to the prior dose. In some embodiments, administration of the specific inhibitor of IL-6, the specific inhibitor of TNFα, or the SMAD7 antisense oligonucleotide to the patient is repeated until the levels of one or more biomarkers, e.g., p-SMAD3 or TGF-β1, reach normal levels.

The invention also provides methods of treating or managing celiac disease in a patient having below normal levels of TGF-β signaling activity, where the amount of a specific inhibitor of IL-6, a specific inhibitor of TNFα, or a SMAD7 antisense oligonucleotide administered to the patient is increased until TGF-β signaling activity levels in the patient increase. In such embodiments, levels of the specific inhibitor of IL-6, the specific inhibitor of TNFα, or SMAD7 antisense oligonucleotide administered to the patient may be increased until the level of TGF-β signaling activity in the patient increases to about a normal level of TGF-β signaling activity or an above normal level of TGF-β signaling activity.

In some embodiments, the invention provides a method of monitoring the treatment or management of celiac disease in a patient with celiac disease, that includes analyzing TGF-β signaling activity in the patient following each SMAD7 antisense oligonucleotide administration, specific inhibitor of IL-6 administration, or specific inhibitor of TNFα administration. Utilizing these methods, the absence of an increase in TGF-β signaling activity levels indicates that the treatment or management is not effective. In such embodiments, TGF-β signaling activity levels may be analyzed one time or multiple times, for instance, two times, three times, four times, about five times, about 10 times, about 15 times, about 20 times, or about 30 times, after each administration of SMAD7 antisense oligonucleotide, specific inhibitor of IL-6 administration, or specific inhibitor of TNFα administration. Furthermore, the timing of the measurement of TGF-β signaling activity levels may vary with respect to the time of SMAD7 oligonucleotide administration, specific inhibitor of IL-6 administration, or specific inhibitor of TNFα administration such that TGF-β signaling activity levels may be analyzed immediately after, about 1 hour after, about 3 hours after, about 6 hours after, about 12 hours after, about 1 day after, about 3 days after, about 1 week after, about 2 weeks after, and/or about 1 month after SMAD7 antisense oligonucleotide administration, specific inhibitor of IL-6 administration, or specific inhibitor of TNFα administration.

In order to determine levels of a biomarker or analyte, for example, p-SMAD3 or TGF-β, in a patient having celiac disease using the methods described herein, a sample may be obtained from the patient. Therefore, in some embodiments of the invention, the level of TGF-β signaling activity, for example the level of Transforming Growth Factor-β1 (TGF-β1), Transforming Growth Factor-β2 (TGF-β2), Transforming Growth Factor-β3 (TGF-β3), Mothers Against Decapentaplegic Homolog 2 (SMAD2), Mothers Against Decapentaplegic Homolog 3 (SMAD3), Mothers Against Decapentaplegic Homolog 4 (SMAD4), phosphorylated SMAD2 (p-SMAD2), or phosphorylated SMAD3 (p-SMAD3) in the patient having celiac disease is determined in a sample obtained from the patient having celiac disease. In some embodiments, the method also includes determining the level of one or more additional analytes, including inflammatory cytokines, for example Tumor Necrosis Factor α (TNFα), Interleukin-6 (IL-6), Interleukin-25 (IL-25), or Interleukin-15 (IL-15). Thus, in some embodiments of the invention, the method includes determining a level, or multiple levels, of one or more analytes in the patient having celiac disease.

Samples containing analytes of interest, for example, TGF-β1, TGF-β2, TGF-β3, SMAD2, SMAD3, SMAD4, p-SMAD2, p-SMAD3, TNFα, IL-6, IL-25, and/or IL-15, obtained from the patient having celiac disease, may include blood, serum, plasma, or intestinal tissue samples, for example small intestinal tissue samples, for example, duodenal or jejunal samples. Samples may also include tissue samples such as, but not limited to, gastrointestinal, mucosal, submucosal, intestinal, esophageal, ileal, rectal, or lymphatic samples. Samples may also include cellular samples, for examples, mucosal cell samples, for example, lamina propria mononuclear cell samples. Levels of analytes of interest in a sample from a patient having celiac disease may be determined using various assays. For example, in methods of the invention, the level of TGF-β1, TGF-β2, TGF-β3, SMAD2, SMAD3, SMAD4, p-SMAD2, p-SMAD3, TNFα, IL-6, IL-25, and/or IL-15 and/or another analyte may be determined by immunochemistry, for example, by an enzyme-linked immunosorbent assay (ELISA), or by nucleotide analysis.

It will be appreciated that the SMAD7 antisense oligonucleotide, the IL-6 antisense oligonucleotide, and/or the TNFα antisense oligonucleotide administered to the patient having celiac disease in methods of the invention described herein, may be administered by various administration routes. In various embodiments, the SMAD7 antisense oligonucleotide, the IL-6 antisense oligonucleotide, and/or the TNFα antisense oligonucleotide may be administered by one or several routes, including orally, topically, parenterally, e.g., by subcutaneous injection, by inhalation spray, or rectally. The term parenteral as used herein includes subcutaneous injections, intrapancreatic administration, and intravenous, intramuscular, intraperitoneal, and intrasternal injection or infusion techniques. In a preferred embodiment, the SMAD7 antisense oligonucleotide may be administered orally to the patient having celiac disease.

The contemplated invention provides methods that include administration of a SMAD7 antisense oligonucleotide capable of targeting SMAD7 RNA for degradation, interfering with RNA splicing or preventing SMAD7 gene expression or protein translation. The contemplated SMAD7 antisense oligonucleotide of the invention may target various regions of the human SMAD7 mRNA for binding. For example, the SMAD7 antisense oligonucleotide may target nucleotides 108-128 of human SMAD7 mRNA (SEQ ID NO: 1). In some embodiments, the SMAD7 antisense oligonucleotide may target nucleotides 403, 233, 294, 295, 296, 298, 299 or 533 of the human SMAD7 sequence (SEQ ID NO: 1). The human SMAD7 mRNA sequence is the sequence of NCBI Reference Sequence: NM_005904.3 (SEQ ID NO: 1).

The sequence of the contemplated SMAD7 antisense oligonucleotide may be selected from multiple sequences capable of targeting SMAD7 RNA. For example, in some embodiments of the invention, the SMAD7 antisense oligonucleotide comprises the nucleotide sequence of SEQ ID NO: 2 (5′-GTCGCCCCTTCTCCCCGCAGC-3′). In some embodiments of the invention, the antisense oligonucleotide is a phosphorothioate antisense oligonucleotide, i.e., an oligonucleotide where at least some of the internucleotide linkages are phosphorothioate linkages, suitable for delivery to cells of a patient. Additionally, antisense oligonucleotides of the invention may include modified nucleotides, for example, nucleotides containing modified bases, for example, 5-methyl-2′-deoxycytidine. For example, in some embodiments, the SMAD7 antisense oligonucleotide is a SMAD7 phosphorothioate antisense oligonucleotide comprising the following sequence: 5′-GTXGCCCCTTCTCCCXGCAG-3′ (SEQ ID NO: 3) wherein X is a nucleotide comprising 5-methyl-2′-deoxycytidine and wherein the internucleotide linkages are phosphorothioate linkages. In some embodiments, the SMAD7 antisense oligonucleotide is a SMAD7 phosphorothioate antisense oligonucleotide comprising the following sequence: 5′-GTXGCCCCTTCTCCCXGCAGC-3′ (SEQ ID NO: 4) wherein X is a nucleotide comprising 5-methyl-2′-deoxycytidine and wherein the internucleotide linkages are phosphorothioate linkages. In a particular embodiment, the contemplated antisense oligonucleotide is an antisense oligonucleotide (referred to herein as “Mongersen”) comprising the free acid form, the salt form, or the anionic form without a counterion of SEQ ID NO: 4, wherein each of the 20 internucleotide linkages is an O,O-linked phosphorothioate linkage and shown in FIG. 1. In some embodiments, the phosphorothioate backbone of SEQ ID NO: 4 can be fully or partially protonated to form an acidic form of SEQ ID NO: 4. In some embodiments, the contemplated salts of SEQ ID NO: 4 include those that are fully neutralized, e.g., each phosphorothioate linkage is associated with an ion such as Na⁺. In some embodiments the salts of SEQ ID NO: 4 are only partially neutralized, e.g., less than all phosphorothioate linkages are associated with an ion (e.g., less than 99%, less than 95%, less than 90%, less than 85%, less than 80%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 3%, or less than 1% are neutralized).

In some embodiments of the invention, a method of treating celiac disease, preventing celiac disease, or preventing collagen deposition in a patient includes administering a pharmaceutical composition, for example, a pharmaceutical composition comprising a specific inhibitor of SMAD7, a specific inhibitor of IL-6, or a specific inhibitor of TNFα (for example, a SMAD7 antisense oligonucleotide, an IL-6 antisense oligonucleotide, or a TNFα antisense oligonucleotide), and a pharmaceutically acceptable carrier. In some embodiments, the pharmaceutical composition is administered parenterally. In some embodiments, the pharmaceutical composition is administered orally. In some embodiments, the pharmaceutical composition includes an enteric coating, for example, an enteric coating comprising an ethylacrylate-methacrylic acid copolymer.

In embodiments of the invention, methods of treating celiac disease, preventing celiac disease, or preventing collagen deposition in a patient include administering varying amounts of a SMAD7 antisense oligonucleotide, an IL-6 antisense oligonucleotide, or a TNFα antisense oligonucleotide or a pharmaceutical composition comprising a SMAD7 antisense oligonucleotide, an IL-6 antisense oligonucleotide, or a TNFα antisense oligonucleotide. In some embodiments, methods of the invention include administering at least 1 μg, at least 5 μg, at least 10 μg, at least, 20 μg, at least, 30 μg, at least, 40 μg, at least, 50 μg, at least, 60 μg, at least, 70 μg, at least, 80 μg, at least, 90 or at least 100 μg of the antisense oligonucleotide. In some embodiments, methods of the invention include administering from 35 mg to 500 mg, from 1 mg to 10 mg, from 10 mg to 20 mg, from 20 mg to 30 mg, from 30 mg to 40 mg, from 40 mg to 50 mg, from 50 mg to 60 mg, form 60 mg to 70 mg, from 70 mg to 80 mg, from 80 mg to 90 mg, from 90 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 600 mg, from 600 mg to 700 mg, from 700 mg to 800 mg, from 800 mg to 900 mg, from 900 mg to 1 g, from 1 mg to 50 mg, from 20 mg to 40 mg, or from 1 mg to 500 mg of the antisense oligonucleotide.

The invention also relates in part to the fact that levels of one or more inhibitors of TGF-β signaling activity and levels of one or more inflammatory cytokines in a patient with celiac disease correlate with celiac disease state and can be used as a means for monitoring disease state and managing responsiveness to celiac disease treatment with an anti-SMAD7 therapy, an anti-IL6 therapy, and/or an anti-TNFα therapy. One can use monitoring and analysis of levels of SMAD7, IL-6, and/or TNFα in a patient with celiac disease to determine appropriate levels of anti-SMAD7 therapy, anti-IL6 therapy, and/or an anti-TNFα therapy administration and to regulate and adjust anti-SMAD7 therapy treatment, anti-IL6 therapy treatment, and/or an anti-TNFα therapy treatment. As such, the invention also relates to methods of preventing, managing, or treating celiac disease in a patient by administering a therapy, for example, an anti-SMAD7 therapy (for example, a SMAD7 antisense oligonucleotide), an anti-IL-6 therapy (for example, a specific inhibitor of IL-6), or an anti-TNFα therapy (for example, a specific inhibitor of TNFα), and adjusting administration of the anti-SMAD7 therapy, anti-IL-6 therapy, or anti-TNFα therapy based on levels of one or more analytes. Such analytes include factors that inhibit TGF-β signaling, e.g., SMAD7, and, inflammatory cytokines associated with celiac disease, for example, IL-6, and TNFα.

For example, in some embodiments, the invention relates to a method for treating or managing celiac disease in a patient having celiac disease, wherein the method comprises (a) administering to the patient an initial dose of a SMAD7 antisense oligonucleotide, a specific inhibitor of IL-6, or a specific inhibitor of TNFα; (b) analyzing the level of SMAD7, IL-6, and/or TNFα in the patient; and (c) if the level of SMAD7, IL-6, and/or TNFα is above normal levels of SMAD7, IL-6, and/or TNFα, then administering to the patient a subsequent dose that is greater than or equal to the initial dose, or, if the level SMAD7, IL-6, and/or TNFα is below normal levels of SMAD7, IL-6, and/or TNFα, then administering to the patient a subsequent dose that is equal to or smaller than the initial dose.

In some embodiments the invention relates to a method for treating or managing celiac disease in a patient having celiac disease, wherein the method comprises (a) analyzing the level of SMAD7, IL-6, and/or TNFα in the patient; and (b) if the level of SMAD7, IL-6, and/or TNFα is above normal levels of SMAD7, IL-6, and/or TNFα, then administering to the patient an initial dose of a SMAD7 antisense oligonucleotide, a specific inhibitor of IL-6, or a specific inhibitor of TNFα. In some embodiments the method further comprises the steps of (c) analyzing the level of SMAD7, IL-6, and/or TNFα in the patient after said administering step; and (d) if the level of SMAD7, IL-6, and/or TNFα is above normal levels of SMAD7, IL-6, and/or TNFα then administering to the patient a subsequent dose of the SMAD7 antisense oligonucleotide, specific inhibitor of IL-6, or specific inhibitor of TNFα that is greater than or equal to the initial dose, or, if the level of SMAD7, IL-6, and/or TNFα is below normal levels of SMAD7, IL-6, and/or TNFα then administering to the patient a subsequent dose of the SMAD7 antisense oligonucleotide, specific inhibitor of IL-6, or specific inhibitor of TNFα that is equal to or smaller than the initial dose. In yet other embodiments, the method comprising steps (a) and (b) further comprises a step (c) analyzing the level of SMAD7, IL-6, and/or TNFα in the patient after said administering step and (d) if the level of SMAD7, IL-6, and/or TNFα is decreased after said administration step compared to the level of SMAD7, IL-6, and/or TNFα before said administration step, then administering to the patient a subsequent dose that is the same as the initial dose or smaller than the initial dose, or, if the level of SMAD7, IL-6, and/or TNFα is unchanged or increased after said administration step compared to the level of SMAD7, IL-6, and/or TNFα before said administration step, then administering to the patient a subsequent dose that is the same as the initial dose or greater than the initial dose or terminating the treatment.

In other embodiments, the invention relates to a method for treating or managing celiac disease in a patient having celiac disease, wherein the method comprises: (a) establishing a control level of SMAD7, IL-6, and/or TNFα for the patient; (b) administering to the patient an initial dose of a SMAD7 antisense oligonucleotide, a specific inhibitor of IL-6, and/or a specific inhibitor of TNFα; (c) analyzing the level of SMAD7, IL-6, and/or TNFα in the patient; and (d) if the level of SMAD7, IL-6, and/or TNFα is lower than the control level, then administering to the patient a subsequent dose that is the same as the initial dose or smaller than the initial dose, or, if the level of SMAD7, IL-6, and/or TNFα is unchanged or increased compared to the control level, then administering to the patient a subsequent dose that is the same as the initial dose or greater than the initial dose or terminating the treatment.

In some embodiments of the invention, the second level of SMAD7, IL-6, and/or TNFα is higher than the first level of SMAD7, IL-6, and/or TNFα. For example, in some embodiments, the second level of SMAD7, IL-6, and/or TNFα is about 10% higher, about 20% higher, about 30% higher, about 40% higher, about 50% higher, about 60% higher, about 70% higher, about 80% higher, about 90% higher, about 100% higher, or more than the first level of SMAD7, IL-6, and/or TNFα. In some embodiments, the second level of SMAD7, IL-6, and/or TNFα is about 10% to about 20% higher, about 20% to about 30% higher, about 30% to about 40% higher, about 40% to about 50% higher, about 50% to about 60% higher, about 60% to about 70% higher, about 70% to about 80% higher, about 80% to about 90% higher, or about 90% to about 100% higher than the first level of SMAD7, IL-6, and/or TNFα. Alternatively, in some embodiments, the second level of SMAD7, IL-6, and/or TNFα is lower than the first level of SMAD7, IL-6, and/or TNFα. For example, in some embodiments, the second level of SMAD7, IL-6, and/or TNFα is about 10% lower, about 20% lower, about 30% lower, about 40% lower, about 50% lower, about 60% lower, about 70% lower, about 80% lower, about 90% lower, or about 100% lower than the first level of SMAD7, IL-6, and/or TNFα. In some embodiments, the second level of SMAD7, IL-6, and/or TNFα is about 10% to about 20% lower, about 20% to about 30% lower, about 30% to about 40% lower, about 40% to about 50% lower, about 50% to about 60% lower, about 60% to about 70% lower, about 70% to about 80% lower, about 80% to about 90% lower, or about 90% to about 100% lower than the first level of SMAD7, IL-6, and/or TNFα.

In some embodiments of the invention, the level of SMAD7, IL-6, and/or TNFα is higher than the normal level of SMAD7, IL-6, and/or TNFα. For example, in some embodiments, the level of SMAD7, IL-6, and/or TNFα is about 10% higher, about 20% higher, about 30% higher, about 40% higher, about 50% higher, about 60% higher, about 70% higher, about 80% higher, about 90% higher, about 100% higher, or more than the normal level of SMAD7, IL-6, and/or TNFα. In some embodiments, the level of SMAD7, IL-6, and/or TNFα is about 10% to about 20% higher, about 20% to about 30% higher, about 30% to about 40% higher, about 40% to about 50% higher, about 50% to about 60% higher, about 60% to about 70% higher, about 70% to about 80% higher, about 80% to about 90% higher, or about 90% to about 100% higher than the normal level of SMAD7, IL-6, and/or TNFα. In some embodiments, the level of SMAD7, IL-6, and/or TNFα is lower than the normal level of SMAD7, IL-6, and/or TNFα. For example, in some embodiments, the level of SMAD7, IL-6, and/or TNFα is about 10% lower, about 20% lower, about 30% lower, about 40% lower, about 50% lower, about 60% lower, about 70% lower, about 80% lower, about 90% lower, or about 100% lower than the normal level of SMAD7, IL-6, and/or TNFα. In some embodiments, the second level of SMAD7, IL-6, and/or TNFα is about 10% to about 20% lower, about 20% to about 30% lower, about 30% to about 40% lower, about 40% to about 50% lower, about 50% to about 60% lower, about 60% to about 70% lower, about 70% to about 80% lower, about 80% to about 90% lower, or about 90% to about 100% lower than the normal level of SMAD7, IL-6, and/or TNFα.

In some embodiments, treatment is adjusted based on a change in the level of SMAD7, IL-6, and/or TNFα, for instance, a decrease in the level of SMAD7, IL-6, and/or TNFα following administration of an anti-SMAD7 therapy, an anti-IL6 therapy, and/or an anti-TNFα therapy. For example, in some embodiments, if the level of SMAD7, IL-6, and/or TNFα is at least 10%, at least 20%, at least 30%, at least 40%, at least 50%, or at least 70% decreased after an administration step compared to the level of SMAD7, IL-6, and/or TNFα before the administration step, then the method calls for administering to the patient a subsequent dose that is the same as the initial dose or smaller than the initial dose.

In embodiments of the invention described herein, if SMAD7, IL-6, and/or TNFα levels in a patient having celiac disease are above normal levels or a control level following an initial administration step (a) or (b), the subsequent dose administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, at least about 160 mg/day, at least about 170 mg/day, at least about 180 mg/day, at least about 190 mg/day, or at least about 200 mg/day greater than the initial dose.

Alternatively, in some embodiments, if SMAD7, IL-6, and/or TNFα levels in a patient having celiac disease are below a control level or normal levels following an initial administration step (a) or (b), the subsequent dose administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, or at least about 100 mg/day smaller than the initial dose.

In embodiments of the invention the amount of the initial dose may vary. For instance, in some embodiments, the initial dose is less than 100 mg/day, less than 90 mg/day, less than 80 mg/day, less than 70 mg/day, less than 60 mg/day, less than 50 mg/day, less than 40 mg/day or less than 30 mg/day. In some embodiments, the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day. In some embodiments, the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day. In some embodiments, the initial dose is 10 mg/day, 40 mg/day, 80 mg/day, or 160 mg/day. In some embodiments, the initial dose administered in an initial administration step (a) or (b) is between about 10 mg/day and 100 mg/day, about 5 mg/day and 200 mg/day, about 10 mg/day and 50 mg/day, about 50 mg/day and 100 mg/day, and about 100 mg/day and about 200 mg/day, and the subsequent dose administered in a step (c) or (d) is between about 30 mg/day and 200 mg/day, about 5 mg/day and 30 mg/day, about 20 mg/day and 50 mg/day, about 50 mg/day and 100 mg/day, or about 100 mg/day and 200 mg/day.

According to methods of the invention, a change in SMAD7, IL-6, and/or TNFα levels observed after an initial administration step (of a specific inhibitor of IL-6, a specific inhibitor of TNFα, or a SMAD7 antisense oligonucleotide) compared to SMAD7, IL-6, and/or TNFα levels prior to the administration step can be compared, for example, as a change in percent of SMAD7, IL-6, and/or TNFα levels, to determine the amount of a subsequent dose of the specific inhibitor of IL-6, the specific inhibitor of TNFα, or the SMAD7 antisense oligonucleotide to be administered to a patient with celiac disease. For example, in some embodiments, if the level of SMAD7, IL-6, and/or TNFα is at least 10%, at least 20%, at least 30%, at least 40%, at least 50%, at least 60%, at least 70%, at least 80%, at least 90%, or at least 95% increased after said administration step (e.g., an administration step (b)) compared to the level of TGF-β signaling activity before said administration step, then the method includes a step (e.g., an administration step (d)) of administering to the patient a subsequent dose that is the same as the initial dose or greater than the initial dose.

The invention also relates to the discovery that a decrease in the level of SMAD7, IL-6, and/or TNFα relative to a level of SMAD7, IL-6, and/or TNFα before treatment may be used as an indication that the patient is likely to experience full or partial clinical amelioration of celiac disease. A decrease in levels of SMAD7, IL-6, and/or TNFα may also be used to determine the length of time during which the patient is likely to experience clinical amelioration. For example, in some embodiments, the method includes determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of celiac disease for a time period of at least 1 week, at least 2 weeks, at least 3 weeks, at least 4 weeks, at least 6 weeks or at least 8 weeks, if the level of SMAD7, IL-6, and/or TNFα after said administering step is decreased at least 10%, at least 20%, at least 30%, at least 40%, at least 50%, at least 60%, or at least 70% compared to the level of SMAD7, IL-6, and/or TNFα before the administration step.

In some embodiments of the invention, administration of any of an anti-SMAD7 therapy, an anti-IL6 therapy, and/or an anti-TNFα therapy is increased if levels of SMAD7, IL-6, and/or TNFα in a patient are above normal levels of SMAD7, IL-6, and/or TNFα. For example, in some embodiments, if the level of SMAD7, IL-6, and/or TNFα is above normal levels of SMAD7, IL-6, and/or TNFα, then the method includes administering to the patient a subsequent dose that is greater than the initial dose. Alternatively, in some embodiments, if the level of SMAD7, IL-6, and/or TNFα is below normal levels of SMAD7, IL-6, and/or TNFα then the method includes the step of administering to the patient a subsequent dose that is smaller than the initial dose of an anti-SMAD7 therapy, an anti-IL6 therapy, and/or an anti-TNFα therapy.

In some embodiments, treatment may be terminated if the level of anti-SMAD7 therapy, anti-IL6 therapy, and/or anti-TNFα therapy for administration equals or surpasses a dose that results in unacceptable side effects, for example, a dose that is clinically effective but results in unacceptable levels of toxicity. In some embodiments, if the subsequent dose of an anti-SMAD7 therapy, an anti-IL6 therapy, and/or an anti-TNFα therapy is equal to or greater than the maximum tolerated dose (MTD), then the method includes the step of terminating the treatment.

In some embodiments, the level of an inhibitor of TGF-β signaling, or an inflammatory cytokine associated with celiac disease will be measured at one or more time points following administration of an anti-SMAD7 therapy, an anti-IL6 therapy, and/or an anti-TNFα therapy. For example, in some embodiments the level of SMAD7, IL-6, and/or TNFα is analyzed at least 1 day, at least 3 days, at least 5 days, at least 1 week, at least 2 weeks, at least 3 weeks, at least 1 month, at least 2 months, at least 4 months, or at least 6 months after an administration step of the method. In some embodiments, the level of SMAD7, IL-6, and/or TNFα is analyzed immediately after an administration step. In some embodiments, the level of SMAD7, IL-6, and/or TNFα is analyzed about 15 days or about 28 days after said administration step.

Normal levels or a control level of an inhibitor of TGF-β signaling activity (e.g., SMAD7) or an inflammatory cytokine associated with celiac disease (e.g., IL-6 and TNFα) may be determined based on numerical reference values or with respect to levels of those analytes in a healthy control group. For instance, in some embodiments, a control level or normal levels of SMAD7, IL-6, and/or TNFα is analyzed by measuring the concentration of SMAD7, IL-6, and/or TNFα. Concentrations of SMAD7, IL-6, and/or TNFα used to determine a control level or normal levels of SMAD7, IL-6, and/or TNFα may be about 0.01 pg/ml, about 0.1 pg/ml, about 0.2 pg/ml, about 0.3 pg/ml, about 0.4 pg/ml, about 0.5 pg/ml, about 0.6 pg/ml, about 0.7 pg/ml, about 0.8 pg/ml, about 0.9 pg/ml, about 1.0 pg/ml, about 1.1 pg/ml, about 1.2 pg/ml, about 1.3 pg/ml, about 1.4 pg/ml, about 1.5 pg/ml, about 1.6 pg/ml, about 1.7 pg/ml, about 1.8 pg/ml, about 1.9 pg/ml, about 2 pg/ml, about 3 pg/ml, about 4 pg/ml, about 5 pg/ml, about 6 pg/ml, about 7 pg/ml, about 8 pg/ml, about 9 pg/ml, about 10 pg/ml, about 11 pg/ml, about 12 pg/ml, about 13 pg/ml, about 14 pg/ml, about 15 pg/ml, about 16 pg/ml, about 17 pg/ml, about 17.5 pg/ml, about 18 pg/ml, about 19 pg/ml, about 20 pg/ml, about 22.5 pg/ml, about 25 pg/ml, about 30 pg/ml, or about 35 pg/ml. In other embodiments of the invention, a control level or normal levels of SMAD7, IL-6, and/or TNFα are defined as median levels of SMAD7, IL-6, and/or TNFα in a healthy control group. A healthy control group may be defined based on various criteria related to genetic background, habits, and physical attributes matched to the same set of criteria in the patient. For instance, in some embodiments, the healthy control group and the patient having celiac disease are matched with respect to age, gender, ethnic origin, smoking habits, dietary habits, body-mass index (BMI), recreational drug use, medical drug use, drug use related to celiac disease, and/or exercise habits. Other factors that can be matched between the patient and control group include, but are not limited to, clinical criteria (e.g., severity of celiac disease-related symptoms), metabolism, celiac disease patient's personal disease history, genetic factors, celiac disease patient's family disease history, exposure to environmental factors (e.g., pollutants, toxins, allergens), and life-style (e.g., urban, suburban, or rural place of work and/or domicile). For example, in some embodiments, normal levels of SMAD7, IL-6, and/or TNFα are median levels of SMAD7, IL-6, and/or TNFα in a healthy control group. In some embodiments, the control level of SMAD7, IL-6, and/or TNFα is a median level of SMAD7, IL-6, and/or TNFα in a healthy control group.

In some embodiments of the invention, the amount of a subsequent dose administered to a patient in a method of the invention may be adjusted based on the level of SMAD7, IL-6, and/or TNFα compared to a normal level or a control level of SMAD7, IL-6, and/or TNFα. A subsequence dose may be administered with respect to the amount of an initial dose. For example, in some embodiments, if SMAD7, IL-6, and/or TNFα levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose. In some embodiments, if SMAD7, IL-6, and/or TNFα levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

Similarly, in some embodiments, if SMAD7, IL-6, and/or TNFα levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose. In some embodiments, if SMAD7, IL-6, and/or TNFα levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose. In some embodiments, the initial dose is between about 10 mg/day and 100 mg/day and the subsequent dose is between about 30 mg/day and 200 mg/day.

In some embodiments, the invention is drawn to a method for treating or managing celiac disease in a patient with celiac disease having above normal SMAD7, IL-6, and/or TNFα levels following administration of a dose of a SMAD7 antisense oligonucleotide, a specific inhibitor of IL-6, or a specific inhibitor of TNFα, wherein the method includes administering to the patient a further dose of the oligonucleotide or specific inhibitor that is greater than or equal to the prior dose. Alternatively, in some embodiments, the invention is drawn to a method for treating or managing celiac disease in a patient with celiac disease having below normal SMAD7, IL-6, and/or TNFα levels following administration of a dose of SMAD7 antisense oligonucleotide, a specific inhibitor of IL-6, or a specific inhibitor of TNFα, wherein the method includes administering to the patient a further dose of the oligonucleotide or specific inhibitor that is less than or equal to the prior dose.

In some embodiments, the invention is drawn to a method of treating or managing celiac disease in a patient with celiac disease having above normal SMAD7, IL-6, and/or TNFα levels, wherein the method includes administering to the patient a dose of a SMAD7 antisense oligonucleotide, a specific inhibitor of IL-6, or a specific inhibitor of TNFα. In some embodiments, the administering is repeated until a SMAD7, IL-6, and/or TNFα level reaches a normal level.

In some embodiments, the invention is drawn to a method of monitoring the treatment or management of celiac disease in a patient with celiac disease, wherein the method includes analyzing SMAD7, IL-6, and/or TNFα levels in the patient following each administration of SMAD7 antisense oligonucleotide, specific inhibitor of IL-6, or specific inhibitor of TNFα, wherein the absence of an decrease in SMAD7, IL-6, and/or TNFα levels indicates that the treatment or management is not effective. In such embodiments, analyte levels may be analyzed multiple times and/or at different time points following administration of a SMAD7 antisense oligonucleotide, specific inhibitor of IL-6, or specific inhibitor of TNFα. For example, in some embodiments, SMAD7, IL-6, and/or TNFα levels are analyzed one time, two times, three times, four times, about five times, about 10 times, about 15 times, about 20 times, or about 30 times after each administration of SMAD7 antisense oligonucleotide, specific inhibitor of IL-6, or specific inhibitor of TNFα. In some embodiments, SMAD7, IL-6, and/or TNFα levels are analyzed immediately after, about 1 hour after, about 3 hours after, about 6 hours after, about 12 hours after, about 1 day after, about 3 days after, about 1 week after, about 2 weeks after, and/or about 1 month after administration of the SMAD7 antisense oligonucleotide, specific inhibitor of IL-6, or specific inhibitor of TNFα.

In some embodiments, the invention relates to a method of treating or managing celiac disease in a patient with celiac disease having above normal levels of SMAD7, IL-6, and/or TNFα, that includes increasing the amount of a SMAD7 antisense oligonucleotide, specific inhibitor of IL-6, or specific inhibitor of TNFα administered to the patient until SMAD7, IL-6, and/or TNFα levels in the patient decrease. In some embodiments, the level of SMAD7, IL-6, and/or TNFα decreases to about a normal level of SMAD7, IL-6, and/or TNFα or a below normal level of SMAD7, IL-6, and/or TNFα.

Embodiments of the invention also relate to use of SMAD7 antisense oligonucleotides, specific inhibitors of IL-6, and specific inhibitors of TNFα. For example, some embodiments relate to a SMAD7 antisense oligonucleotide, a specific inhibitor of IL-6, or a specific inhibitor of TNFα for use in a method for treating or managing celiac disease in a patient having celiac disease, wherein the method includes analyzing the level of SMAD7, IL-6, and/or TNFα in the patient to determine appropriate levels of SMAD7 antisense oligonucleotide, specific inhibitor of IL-6, or specific inhibitor of TNFα administration. In some embodiments, the invention relates to a method comprising the steps of: (a) administering to the patient an initial dose of the SMAD7 antisense oligonucleotide, specific inhibitor of IL-6, or specific inhibitor of TNFα; (b) analyzing the level of a SMAD7, IL-6, and/or TNFα in the patient; and (c) if the level of SMAD7, IL-6, and/or TNFα is above normal levels of SMAD7, IL-6, and/or TNFα, then administering to the patient a subsequent dose of the SMAD7 antisense oligonucleotide, specific inhibitor of IL-6, or specific inhibitor of TNFα that is greater than or equal to the initial dose. Alternatively, if the level of SMAD7, IL-6, and/or TNFα is below normal levels of SMAD7, IL-6, and/or TNFα then the method includes the step of administering to the patient a subsequent dose of the SMAD7 antisense oligonucleotide, specific inhibitor of IL-6, or specific inhibitor of TNFα that is equal to or smaller than the initial dose. In yet other embodiments, the invention relates to a SMAD7 antisense oligonucleotide, specific inhibitor of IL-6, or specific inhibitor of TNFα for use in a method for treating or managing celiac disease in a patient having celiac disease, wherein the method comprises (a) analyzing the level of SMAD7, IL-6, and/or TNFα in the patient; and (b) if the level of SMAD7, IL-6, and/or TNFα is above normal levels of SMAD7, IL-6, and/or TNFα, then administering to the patient an initial dose of the SMAD7 antisense oligonucleotide, the specific inhibitor of IL-6, or the specific inhibitor of TNFα. The SMAD7 antisense oligonucleotides, specific inhibitors of IL-6, and specific inhibitors of TNFα for use as medicaments are also provided. In particular, the SMAD7 antisense oligonucleotides, specific inhibitors of IL-6, and specific inhibitors of TNFα are provided for use in the treatment of celiac disease, preferably wherein treatment is by a method as described herein. Furthermore, the use of the SMAD7 antisense oligonucleotides, specific inhibitors of IL-6, and specific inhibitors of TNFα in the manufacture of medicaments for use in the methods described herein is provided.

Levels of SMAD7, IL-6, and/or TNFα may be analyzed by measuring gene products of the SMAD7, IL-6, and/or TNFα genes, including mRNA and protein levels of the SMAD7, IL-6, and/or TNFα genes. For example, in some embodiments, SMAD7, IL-6, and/or TNFα is analyzed by measuring the concentration of SMAD7 protein or SMAD7 mRNA, IL-6 protein or IL-6 mRNA, and/or TNFα protein or TNFα mRNA. Levels of SMAD7, IL-6, and/or TNFα mRNA or protein may be determined using various detection methods. For example, in some embodiments, the level of SMAD7, IL-6, and/or TNFα is determined by immunochemistry or by nucleotide analysis. In some embodiments, the level of SMAD7, IL-6, and/or TNFα is determined by performing an enzyme-linked immunosorbent assay (ELISA). In some embodiments, methods of the disclosure include analyzing the level of SMAD7, IL-6, and/or TNFα, where the level of SMAD7, IL-6, and/or TNFα is analyzed by measuring the concentration of SMAD7 protein, IL-6 protein, and/or TNFα protein. For example, in some embodiments, methods of the disclosure include analyzing the level of SMAD7, where SMAD7 is analyzed by measuring the concentration of SMAD7 protein. In some embodiments, the disclosure includes a compound for use in a method for treating or managing celiac disease in a patient having celiac disease (for example, a SMAD7 antisense oligonucleotide, specific inhibitor of IL-6, or specific inhibitor of TNFα for use in a method for treating or managing celiac disease in a patient having celiac disease), where the method includes analyzing the level of SMAD7 by measuring the concentration of SMAD7 protein.

In addition, methods of the invention may also include a step of analyzing the level of another analyte beyond SMAD7, IL-6, and/or TNFα. For example, in some embodiments, the method includes the step of determining a level of one or more additional analytes in the patient having celiac disease, for example, IL-25 and/or IL-15.

The level of SMAD7, IL-6, and/or TNFα in a patient having celiac disease may be determined by obtaining a sample from the patient. For example, in some embodiments, the level of SMAD7, IL-6, and/or TNFα in the patient having celiac disease is determined in a sample obtained from the patient having celiac disease. In some embodiments, the sample is a blood, serum, plasma, or intestinal tissue sample.

In some embodiments, the anti-SMAD7 therapy, anti-IL-6 therapy, or the anti-TNFα therapy includes, respectively, administration of a SMAD7 antisense oligonucleotide, an IL-6 antisense oligonucleotide, or a TNFα antisense oligonucleotide. In some embodiments, the SMAD7 antisense oligonucleotide, the IL-6 antisense oligonucleotide, or the TNFα antisense oligonucleotide is administered orally to the patient having celiac disease.

The SMAD7 antisense oligonucleotide may comprise any of several SMAD7 antisense oligonucleotides that target various regions of the human SMAD7 mRNA sequence. For example, in some embodiments, the SMAD7 antisense oligonucleotide targets region 108-128 of human SMAD7 (SEQ ID NO: 1). In some embodiments, the SMAD7 antisense oligonucleotide targets nucleotides 403, 233, 294, 295, 296, 298, 299 or 533 of human SMAD7 (SEQ ID NO: 1). In some embodiments, the SMAD7 antisense oligonucleotide comprises the nucleotide sequence of SEQ ID NO: 2 (5′-GTCGCCCCTTCTCCCCGCAGC-3′). In some embodiments, the antisense oligonucleotide is a SMAD7 phosphorothioate antisense oligonucleotide comprising the following sequence: 5′-GTXGCCCCTTCTCCCXGCAG-3′ (SEQ ID NO: 3) wherein X is a nucleotide comprising 5-methyl-2′-deoxycytidine and wherein the internucleotide linkages are phosphorothioate linkages. In some embodiments, the antisense oligonucleotide is a SMAD7 phosphorothioate antisense oligonucleotide comprising the following sequence: 5′-GTXGCCCCTTCTCCCXGCAGC-3′ (SEQ ID NO: 4) wherein X is a nucleotide comprising 5-methyl-2′-deoxycytidine and wherein the internucleotide linkages are phosphorothioate linkages.

### BRIEF DESCRIPTION OF THE DRAWINGS

FIG. 1 shows the molecular structure of Mongersen.

FIG. 2A shows Western blots of SMAD7 and β-actin expression in duodenal tissue protein extracts of a control patient (CTR), a patient with inactive celiac disease (ICD), a patient with active celiac disease (ACD), and a patient with refractory celiac disease (RCD). FIG. 2B shows quantification of SMAD7 signal by densitometric analysis in each sample relative to β-actin expression. FIG. 2C shows quantification of relative SMAD7 mRNA expression in 11 CTR, 9 ICD, 11 ACD, and 7 RCD patients.

FIG. 3A shows epithelial and lamina propria gastrointestinal tissue from patients with Refractory Celiac Disease Type I (RCDI), Refractory Celiac Disease Type II (RCDII), active celiac disease (ACD), inactive celiac disease (ICD), and healthy control (CTR) patients immunostained for SMAD7. FIG. 3A also shows gastrointestinal tissue stained for isotype control (Isotype).

FIG. 3B shows quantification of SMAD7-positive cells in CTR, ICD, ACD, and RCD tissue samples.

FIG. 3C shows quantification of SMAD7-positive cells in RCDI and RCDII tissue samples.

FIG. 4 shows quantification of active TGF-β1 protein levels in gastrointestinal tissue of CTR, ICD, ACD, and RCD patients. N is the number of patients per group.

FIG. 5A shows epithelial and lamina propria gastrointestinal tissue from patients with RCDI, RCDII, ACD, ICD, and CTR patients immunostained for active SMAD2/3 (pSMAD2/3). FIG. 5A also shows gastrointestinal tissue stained for isotype control.

FIG. 5B shows quantification of pSMAD2/3-positive cells in CTR, ICD, ACD, and RCD tissue samples.

FIG. 5C shows quantification of pSMAD2/3-positive cells in RCDI and RCDII tissue samples.

FIG. 5D shows the number of SMAD7-positive and pSMAD2/3-positive cells in individual RCD patient gastrointestinal tissue samples.

FIG. 6A shows three representative sets of Western blots of tissue extract from individual normal duodenal mucosa exposed to either IL-6, TNFα, or IL-15, or left unstimulated. Western blots show expression of SMAD7 or β-actin signal.

FIG. 6B shows quantification of SMAD7 protein signal relative to β-actin control signal in the Western blots of FIG. 6A.

### DETAILED DESCRIPTION

The invention provides methods that are generally useful for treating and managing celiac disease in a patient having celiac disease. The method is particularly useful in terms of managing treatment in a patient being treated with an anti-SMAD7 therapy, such as a SMAD7 antisense oligonucleotide therapy. A SMAD7 antisense oligonucleotide therapy may be any therapy that includes an oligonucleotide that is capable of binding to a SMAD7 mRNA transcript and inducing degradation of the SMAD7 mRNA transcript, preventing splicing of the SMAD7 mRNA transcript, or preventing protein translation of the SMAD7 mRNA transcript.

The method is also useful in terms of managing treatment in a patient being treated with an specific inhibitor of IL-6, such as an IL-6 antisense oligonucleotide therapy, or a specific inhibitor of TNFα, such as a TNFα antisense oligonucleotide therapy. An IL-6 antisense oligonucleotide may be any therapy that includes an oligonucleotide that is capable of binding to an IL-6 mRNA transcript and inducing degradation of the IL-6 mRNA transcript, preventing splicing of the IL-6 mRNA transcript, or preventing protein translation of the IL-6 mRNA transcript. A TNFα antisense oligonucleotide may be any therapy that includes an oligonucleotide that is capable of binding to a TNFα mRNA transcript and inducing degradation of the TNFα mRNA transcript, preventing splicing of the TNFα mRNA transcript, or preventing protein translation of the TNFα mRNA transcript.

Methods of the invention are useful for predicting and determining responsiveness of patients having celiac disease to treatment with a SMAD7 antisense oligonucleotide, a specific inhibitor of IL-6, or a specific inhibitor of TNFα. Thus, methods of the invention can be used to identify patients that are likely to respond to SMAD7 antisense oligonucleotide treatment, specific inhibitor of IL-6 treatment, or specific inhibitor of TNFα treatment as well as patients that are unlikely to respond to SMAD7 antisense oligonucleotide treatment, specific inhibitor of IL-6 treatment, or specific inhibitor of TNFα treatment. The methods described herein are also useful for determining whether a patient is or is not responsive to celiac disease treatment. Generally, methods of the invention can also be used to determine the level or likely level of responsiveness in a patient having celiac disease being treated with a SMAD7 antisense oligonucleotide, a specific inhibitor of IL-6, or a specific inhibitor of TNFα. Based upon a determination of a level of responsiveness or a likely level of responsiveness, administration of the SMAD7 antisense oligonucleotide, the specific inhibitor of IL-6, or the specific inhibitor of TNFα may be initiated, repeated, maintained, increased, decreased, or terminated. Responsiveness may be determined using a number of factors including, but not limited to: analysis of levels or changes in TGF-β signaling activity levels, including levels of biomarkers and/or other analytes (e.g., TGF-β and p-SMAD3), or assessment of symptoms of celiac disease (e.g., weight loss, steatorrhoea, diarrhea, abdominal pain, cramping, bloatedness, abdominal distension, and mouth ulcers).

Similarly, the methods are useful for evaluating efficacy and safety of treatment with a SMAD7 antisense oligonucleotide, a specific inhibitor of IL-6, or a specific inhibitor of TNFα in a patient having celiac disease. For example, methods of the invention may include determining changes in levels of biomarker expression or other indicators or manifestations of disease state that can indicate that treatment with the SMAD7 antisense oligonucleotide, the specific inhibitor of IL-6, or the specific inhibitor of TNFα is effective or not effective to cause partial or complete amelioration of celiac disease. Determining levels or changes in levels of biomarker expression, disease symptoms, tissue, cellular, blood, or systemic levels of the SMAD7 antisense oligonucleotide, the specific inhibitor of IL-6, or the specific inhibitor of TNFα, or indicators of general health may also indicate a worsening of disease state or unsafe drug levels. Assessment of multiple indicators before, during, between, and/or after treatment(s) may be used to monitor disease stage, progression, and severity.

The invention is based in part on the discovery of a relationship between celiac disease state and TGF-β signaling activity and inflammatory cytokine levels. Specifically, the inventors have discovered that TGF-β signaling activity levels, including levels of TGF-β1, TGF-β2, TGF-μ3, SMAD2, SMAD3, SMAD4, p-SMAD2, p-SMAD3, as well as levels of the inflammatory cytokines such as TNFα, IL-6, IL-25, and IL-15 are useful for determining whether a patient is responsive to, likely to be responsive to, not responsive to, or likely not responsive to treatment of celiac disease using a SMAD7 antisense oligonucleotide, a specific inhibitor of IL-6, or a specific inhibitor of TNFα.

Furthermore, TGF-β signaling activity levels can be used to manage disease treatment using a SMAD7 antisense oligonucleotide, a specific inhibitor of IL-6, or a specific inhibitor of TNFα, specifically with respect to dose amount of the SMAD7 antisense oligonucleotide, the specific inhibitor of IL-6, or the specific inhibitor of TNFα. For example, levels of TGF-β signaling activity may be used to determine whether a patient having celiac disease should be given a specific dose amount, for example, a higher dose or a lower dose, of SMAD7 antisense oligonucleotide, a specific inhibitor of IL-6, or a specific inhibitor of TNFα, for example in a subsequent dose, with respect to, for example, a previously administered dose, for example, an initial dose, of SMAD7 antisense oligonucleotide, specific inhibitor of IL-6, or specific inhibitor of TNFα. Thus, administration of a SMAD7 antisense oligonucleotide, a specific inhibitor of IL-6, or a specific inhibitor of TNFα may be adjusted in terms of, for example, dose amount or frequency, with respect to absolute levels of TGF-β signaling activity or relative levels of TGF-β signaling activity in a patient having celiac disease. For instance, administration of a SMAD7 antisense oligonucleotide, specific inhibitor of IL-6, or specific inhibitor of TNFα may be adjusted based on absolute levels of TGF-β signaling activity by comparing absolute levels of TGF-β signaling activity measured in a sample from a patient having celiac disease with a normal level of TGF-β signaling activity, where the normal level of TGF-β signaling activity is, for instance, either a benchmark value or a median level of TGF-β signaling activity in a healthy control group matched to the patient having celiac disease. In some embodiments of the invention, administration of a SMAD7 antisense oligonucleotide, a specific inhibitor of IL-6, or a specific inhibitor of TNFα may be adjusted based on relative levels of TGF-β signaling activity, for instance, based on a comparison of TGF-β signaling activity levels before and after SMAD7 antisense oligonucleotide administration, specific inhibitor of IL-6 administration, or specific inhibitor of TNFα administration, immediately after and later after SMAD7 antisense oligonucleotide administration, specific inhibitor of IL-6 administration, or specific inhibitor of TNFα administration, or during and after SMAD7 antisense oligonucleotide administration specific inhibitor of IL-6 administration, or specific inhibitor of TNFα administration. In some embodiments, the SMAD7 antisense oligonucleotide, specific inhibitor of IL-6, or specific inhibitor of TNFα may be administered multiple times between an initial detection of TGF-β signaling activity levels and a later detection of TGF-β signaling activity levels used to generate the comparison of TGF-β signaling activity levels in the patient sample.

In some embodiments of the invention the celiac disease patient being treated is a patient with below-normal TGF-β signaling activity levels. In some embodiments, a patient is known to have low TGF-β signaling activity levels before treatment. In some embodiments, TGF-β signaling activity levels in the celiac disease patient are determined before treatment, after treatment, before administration of an initial dose of a SMAD7 antisense oligonucleotide, a specific inhibitor of IL-6, or a specific inhibitor of TNFα, after administration of an initial dose of a SMAD7 antisense oligonucleotide, a specific inhibitor of IL-6, or a specific inhibitor of TNFα, before administration of a subsequent dose of a SMAD7 antisense oligonucleotide, a specific inhibitor of IL-6, or a specific inhibitor of TNFα, and/or after administration of a subsequent dose of a SMAD7 antisense oligonucleotide, a specific inhibitor of IL-6, or a specific inhibitor of TNFα.

### Control Levels and Control Samples

A control level of TGF-β signaling activity may be determined by determining the level of TGF-β signaling activity, for example, levels of an analyte such as pSMAD3 or TGF-β1, in a sample (e.g., a blood sample or a small intestinal tissue sample) obtained from the subject prior to treatment with an anti-SMAD7 therapy, an anti-IL-6 therapy, or an anti-TNFα therapy. The control level of TGF-β signaling activity may provide a baseline for monitoring a subject's response to treatment. A control sample may be obtained from the subject on the day the anti-SMAD7 therapy, the anti-IL-6 therapy, or the anti-TNFα therapy is first administered (e.g., Day 1 of a treatment regimen), for example, immediately after administration of at least one anti-SMAD7 therapy, anti-IL-6 therapy, or anti-TNFα therapy. In other embodiments, a control sample may be obtained from a subject one day prior to the start of an anti-SMAD7 therapy, an anti-IL-6 therapy, or an anti-TNFα therapy (e.g., Day 0 of a treatment regimen). Alternatively, a control sample may be obtained from a subject 2, 3, 4, 5, 6, 7 or more days prior to the start of an anti-SMAD7 therapy, an anti-IL-6 therapy, or an anti-TNFα therapy. For example, the increase or decrease in TGF-β signaling activity may be measured prior to treatment (e.g., in a control sample), during treatment, and/or after treatment to monitor a subject's response to therapy, e.g., an anti-SMAD7 therapy, an anti-IL-6 therapy, or an anti-TNFα therapy.

In some embodiments, a control level may be established for a subject based on long-term monitoring of TGF-β signaling activity, for example, of circulating p-SMAD3 or TGF-β1 concentration in the subject. In such instances, it is contemplated that a subject may undergo multiple rounds of treatment with an anti-SMAD7 therapy, an anti-IL-6 therapy, or an anti-TNFα therapy. The circulating analyte concentration detected following multiple rounds of treatment may be compared to a prior control level of TGF-β signaling activity for the subject to determine whether the subject has responded to therapy and/or is likely to respond to further treatment with an anti-SMAD7 therapy, an anti-IL-6 therapy, or an anti-TNFα therapy. In other embodiments, a control or baseline level for a subject may be established based on an average measurement of a circulating analyte concentration determined from multiple baseline samples obtained over time (e.g., obtained over the course of days, weeks, months, or years). Accordingly, any test or assay conducted as disclosed herein may be compared with a previous or established control level and it may not be necessary to obtain a new control sample from the subject for comparison, e.g., if the subject is receiving more than one round of treatment with an anti-SMAD7 therapy, an anti-IL-6 therapy, or an anti-TNFα therapy.

Normal levels of TGF-β signaling activity may be determined based on numerical reference values or with respect to levels of TGF-β signaling activity in a healthy control group.

In other embodiments of the invention, normal levels of TGF-β signaling activity are defined as median levels of TGF-β signaling activity in a healthy control group.

A healthy control group may be defined based on various criteria related to genetic background, habits, and physical attributes matched to the same set of criteria in the patient. For instance, in some embodiments, the healthy control group and the patient having celiac disease are matched with respect to age, gender, ethnic origin, smoking habits, dietary habits, body-mass index (BMI), recreational drug use, medical drug use, drug use related to celiac disease, and/or exercise habits. Other factors that can be matched between the patient and control group include, but are not limited to, clinical criteria (e.g., severity of celiac disease-related symptoms), metabolism, celiac disease patient's personal disease history, genetic factors, celiac disease patient's family disease history, exposure to environmental factors (e.g., pollutants, toxins, allergens), and life-style (e.g., urban, suburban, or rural place of work and/or domicile).

In some embodiments, the control group is the patient receiving a treatment with an SMAD7 antisense oligonucleotide, specific inhibitor of IL-6, or specific inhibitor of TNFα prior to receiving an initial dose of the SMAD7 antisense oligonucleotide, specific inhibitor of IL-6, or specific inhibitor of TNFα. In some embodiments, the patient is a treatment naive patient.

### Data Interpretation

In some embodiments, prior to initial administration of an anti-SMAD7 therapy, an anti-IL-6 therapy, or an anti-TNFα therapy, the level of TGF-β signaling activity in a patient having celiac disease is analyzed and compared to a threshold level. As described herein, a threshold level may be established based on TGF-β signaling activity levels in a healthy control group or a group of celiac disease patients. In general, a threshold level will be elevated with respect to normal TGF-β signaling activity levels, for example median TGF-β signaling activity levels in a healthy control group, or it may fall within the spectrum of TGF-β signaling activity levels in a control group, for example a control group comprised of celiac disease patients.

A subject's responsiveness to treatment with an anti-SMAD7 therapy, an anti-IL-6 therapy, or an anti-TNFα therapy can be interpreted with respect to the control level of TGF-β signaling activity in a sample obtained from the subject prior to treatment. A subject may be identified as sensitive to treatment (e.g., responsive or likely to respond to treatment) with an anti-SMAD7 therapy if there is a decrease in the concentration of TGF-β signaling activity in the sample obtained from the subject compared to the control sample. In some embodiments the sample may be obtained while the subject is receiving an anti-SMAD7 therapy treatment, an anti-IL-6 therapy treatment, or an anti-TNFα therapy treatment. In other embodiments, the sample may be obtained after the subject has stopped receiving treatment, for example, about 1 day, about 7 days (i.e., about 1 week), about 14 days (i.e., about 2 weeks), about 28 days, about 56 days, about 70 days and/or longer, after stopping treatment. In a preferred embodiment, the sample may be obtained about one day after stopping anti-SMAD7 therapy treatment, anti-IL-6 therapy treatment, or anti-TNFα therapy treatment.

In some embodiments, patients receiving an anti-SMAD7 therapy, an anti-IL-6 therapy, or an anti-TNFα therapy, such as, respectively, a SMAD7 antisense oligonucleotide, a specific inhibitor of, or a specific inhibitor of also receive one or more additional celiac disease therapies. In some embodiments, patients receiving the anti-SMAD7 therapy, the anti-IL-6 therapy, or the anti-TNFα therapy and the one or more additional celiac disease therapies can taper the one or more additional celiac disease therapies if they respond to the anti-SMAD7 therapy, anti-IL-6 therapy, or anti-TNFα therapy, e.g., as indicated by increasing TGF-β signaling activity.

Alternatively, a subject may be identified as resistant to treatment (e.g., non-responsive or unlikely to respond) with an anti-SMAD7 therapy, an anti-IL-6 therapy, or an anti-TNFα therapy if there is no change or a decrease in TGF-β signaling activity levels in the sample obtained from the subject, compared to the control level. In one embodiment, the sample may be obtained while the subject is receiving an anti-SMAD7 therapy treatment, an anti-IL-6 therapy treatment, or an anti-TNFα therapy treatment. In other embodiments, the sample may be obtained after the subject has stopped receiving treatment, for example, about 1 day, about 7 days (i.e., about 1 week), about 14 days (i.e., about 2 weeks), about 28 days, about 56 days, about 70 days, and/or longer after stopping treatment. In a preferred embodiment, the sample may be obtained about one day after stopping anti-SMAD7 therapy treatment, anti-IL-6 therapy treatment, or anti-TNFα therapy treatment.

In some embodiments, one or more rescue therapies (e.g., a biologic such as a TNFα inhibitor and/or an immunosuppressive drug) is administered to patients experiencing a worsening of disease during a course of treatment with an anti-SMAD7 therapy, an anti-IL-6 therapy treatment, or an anti-TNFα therapy treatment, e.g., as indicated by decreasing p-SMAD3 or TGF-β1 levels (e.g., >50% decrease in p-SMAD3 or TGF-β1 levels).

Differences in patient TGF-β signaling activity levels and threshold TGF-β signaling activity levels are indicative of a patient's potential responsiveness to anti-SMAD7 therapy, anti-IL-6 therapy treatment, or anti-TNFα therapy treatment. For example, patient TGF-β signaling activity levels that are elevated relative to a threshold TGF-β signaling activity level indicate that a patient may be responsive to anti-SMAD7 therapy, anti-IL-6 therapy treatment, or anti-TNFα therapy treatment. Threshold levels of TGF-β signaling activity can be established using different criteria. In some embodiments, the threshold level of TGF-β signaling activity is determined with respect to normal TGF-β signaling activity levels, for example median TGF-β signaling activity levels, in a control group. Control groups may be comprised of healthy/normal subjects (e.g., a healthy control group) or groups of celiac disease patients.

For instance, in some embodiments, a TGF-β signaling activity threshold level is at least 2-fold, at least 3-fold, at least 5-fold, at least 8-fold, at least 10-fold, at least 20-fold, at least 30-fold, at least 50-fold, at least 80-fold, or at least 100-fold above normal levels. In other embodiments, the TGF-β signaling activity threshold level is in the 50^{th} percentile, 60^{th} percentile, 70^{th} percentile, 80^{th} percentile or 90^{th} percentile of TGF-β signaling activity levels with respect to TGF-β signaling activity levels, for example median TGF-β signaling activity levels, in a group of celiac disease patients. Additionally, in some embodiments, the threshold level of TGF-β signaling activity, for example a level of TGF-β1 or p-SMAD3 is at least or about 1 pg/ml, at least or about 2.5 pg/ml, at least or about 5 pg/ml, at least or about 7.5 pg/ml, at least or about 10 pg/ml, at least or about 12.5 pg/ml, at least or about 15 pg/ml, at least or about 17.5 pg/ml, at least or about 20 pg/ml, at least or about 25 pg/ml, at least or about 30 pg/ml, at least or about 35 pg/ml, at least or about 40.0 mg/L, or at least or about 45.0 mg/L (e.g., as measured in blood serum).

In some embodiments, the control group may consist of the patient receiving an initial dose of a SMAD7 antisense oligonucleotide, a specific inhibitor of IL-6, or a specific inhibitor of TNFα. In some embodiments, normal TGF-β signaling activity levels, or TGF-β signaling activity threshold levels, may be the TGF-β signaling activity baseline levels that are observed in a patient prior to administration of an initial dose of SMAD7 antisense oligonucleotide, specific inhibitor of IL-6, or specific inhibitor of TNFα. TGF-β signaling activity levels can subsequently be monitored in a patient over time, following the administration of the initial dose or of subsequent doses of SMAD7 antisense oligonucleotide, specific inhibitor of IL-6, or specific inhibitor of TNFα to the patient. TGF-β signaling activity levels in the patient following one or more administrations of a SMAD7 antisense oligonucleotide, specific inhibitor of IL-6, or specific inhibitor of TNFα can be compared to the TGF-β signaling activity baseline level in the patient. Dosing regimens for the SMAD7 antisense oligonucleotide, specific inhibitor of IL-6, or specific inhibitor of TNFα can be adjusted, depending on whether TGF-β signaling activity levels in the patient increase, decrease or remain constant relative to the patient's TGF-β signaling activity baseline level.

### Anti-SMAD7, Anti-IL-6, and Anti-TNFα Therapies

The present disclosure is directed in part to methods of treating celiac disease in a patient with an anti-SMAD7 therapy comprising a SMAD7 inhibitor. SMAD7 inhibitors for use as medicaments and for use in treating celiac disease are also disclosed, preferably wherein treatment is by a method as described herein. Furthermore, the use of SMAD7 inhibitors in the manufacture of medicaments for use in the methods described herein is disclosed. SMAD7 inhibitors may include, for example, small binding molecules, e.g., natural and synthetic compounds, antibodies, aptamers, intramers, RNAi (double stranded RNA, siRNA) and SMAD7 antisense oligonucleotides that bind, degrade, or otherwise interfere with SMAD7 stability, production, or function. SMAD7 inhibitors may also comprise truncated and/or mutated SMAD7 molecules which interfere with SMAD7 activity, binding partners, or substrates and which, thereby, inhibit SMAD7 function.

The present disclosure is also directed in part to methods of treating celiac disease in a patient with an anti-IL-6 therapy comprising a specific inhibitor of IL-6. IL-6 inhibitors for use as medicaments and for use in treating celiac disease are also disclosed, preferably wherein treatment is by a method as described herein. Furthermore, the use of IL-6 inhibitors in the manufacture of medicaments for use in the methods described herein is disclosed. Specific inhibitors of IL-6 may include, for example, small binding molecules, e.g., natural and synthetic compounds, antibodies, aptamers, intramers, RNAi (double stranded RNA, siRNA) and IL-6 antisense oligonucleotides that bind, degrade, or otherwise interfere with IL-6 stability, production, or function. IL-6 inhibitors may also comprise truncated and/or mutated IL-6 molecules which interfere with IL-6 activity, binding partners, or substrates and which, thereby, inhibit IL-6 function. Specific inhibitors of IL-6 may inhibit binding of IL-6 to the IL-6 receptor, or inhibit activity or expression of IL-6 signaling cascade components, for example, JAK1, JAK2, JAK3, TYK2, SHC, HCK, STAT1, STAT3, VAV1, GAB1, GRB2, Ras, PI3K, Akt/PKB, IKK, IκB, and NF-κB. Specific inhibitors of IL-6 include tocilizumab (Actemra® and RoActemra®, Hoffmann-La Roche and Chugai, targeting the IL-6 receptor), siltuximab (Sylvant®, Janssen Biotech, Inc.), sarilumab (Regeneron and Sanofi, targeting the IL-6 receptor), olokizumab (CDP6038), elsilimomab (also known as B-E8), clazakizumab (Alder Biopharmaceuticals, Inc.), sirukumab (CNTO 136, Centocor), ALX-0061 (targeting the IL-6 receptor), olokizumab, ARGX-109 (arGEN-X), and FM101 (Femta Pharmaceuticals).

The present disclosure is also directed in part to methods of treating celiac disease in a patient with an anti-TNFα therapy comprising a specific inhibitor of TNFα. TNFα inhibitors for use as medicaments and for use in treating celiac disease are also disclosed, preferably wherein treatment is by a method as described herein. Furthermore, the use of TNFα inhibitors in the manufacture of medicaments for use in the methods described herein is disclosed. Specific inhibitors of TNFα may include, for example, small binding molecules, e.g., natural and synthetic compounds, antibodies, aptamers, intramers, RNAi (double stranded RNA, siRNA) and TNFα antisense oligonucleotides that bind, degrade, or otherwise interfere with TNFα stability, production, or function. Specific inhibitors of TNFα may inhibit binding of TNFα to the TNF receptor type 1 (TNFR1) and/or the TNF receptor type 2 (TNFR2) or inhibit activity or expression of TNFα signaling cascade components. TNFα inhibitors may also comprise truncated and/or mutated TNFα molecules which interfere with TNFα activity, binding partners, or substrates and which, thereby, inhibit TNFα function. Specific inhibitors of TNFα include infliximab (Remicade®, Johnson & Johnson), adalimumab (Humira®, AbbVie), certolizumab pegol (Cimzia®, UCB), golimumab (Simponi®, Janssen), etanercept (Enbrel®, Amgen), thalidomide (Immunoprin®) and its derivatives (e.g., lenalidomide (Revlimid®)), xanthine derivatives (e.g., pentoxifylline), bupropion, and 5-HT_{2A} agonist hallucinogens (e.g., (R)-DOI, TCB-2, LSD and LA-SS-Az).

The present disclosure is also directed in part to methods of treating celiac disease in a patient with a SMAD7 antisense oligonucleotide, an IL-6 antisense oligonucleotide, or a TNFα antisense oligonucleotide. Antisense oligonucleotides are short synthetic oligonucleotide sequences complementary to the messenger RNA (mRNA), which encodes for the target protein (e.g., SMAD7, IL-6, or TNFα). In general, antisense oligonucleotide sequences hybridize to the mRNA producing a double-strand hybrid that can lead to the activation of ubiquitary catalytic enzymes, such as RNase H, which degrades DNA/RNA hybrid strands thus preventing protein translation.

Contemplated IL-6 antisense oligonucleotides may target any region of the IL-6 mRNA, for example, any region of the human IL-6 mRNA (e.g., of SEQ ID NO: 6, NCBI Reference Sequence XM_011515390.2 or SEQ ID NO: 7, NCBI Reference Sequence XM_005249745.4).

Contemplated TNFα antisense oligonucleotides may target any region of the TNFα mRNA, for example, any region of the human TNFα mRNA (e.g., of SEQ ID NO: 8, NCBI Reference Sequence NM_000594.3).

The contemplated SMAD7 antisense oligonucleotide may target any region of the SMAD7 mRNA. In certain embodiments, an anti-SMAD7 antisense oligonucleotide may target site 403, 233, 294, 295, 296, 298, 299, and/or 533 (i.e., nucleotides 403, 233, 294, 295, 296, 298, 299, and 533, respectively) of the human SMAD7 mRNA (e.g., of SEQ ID NO: 1; NCBI Reference Sequence NM_005904.3).

In certain embodiments, an antisense oligonucleotide may be derived from the following anti-SMAD7 antisense oligonucleotide 5′-GTCGCCCCTTCTCCCCGCAGC-3′ (SEQ ID NO: 2).

It is contemplated herein that an antisense oligonucleotide targeting SMAD7 may comprise a mixed-backbone wherein the cytosine residues in a CpG pair are replaced by 5′-methylcytosine (abbreviated as Me-dC). Methylphosphonate linkages may also be placed at the 5′ and/or 3′ ends of an antisense oligonucleotide (abbreviated as MeP).

Exemplary antisense oligonucleotide therapies that target SMAD7 include, but are not limited to, 5′-GTXYCCCCTTCTCCCXYCAG-3′ (SEQ ID NO: 5), wherein X is a nucleotide comprising a nitrogenous base selected from the group consisting of cytosine and 5-methylcytosine or a 2′-O-methylcytosine nucleoside, and wherein Y is a nucleotide comprising a nitrogenous base selected from the group consisting of guanine and 5-methylguanine or a 2′-O-methylguanine nucleoside, provided that at least one of the nucleotides X or Y comprises a methylated nitrogenous base;

5′-GTXGCCCCTTCTCCCXGCAG-3′ (SEQ ID NO: 3), wherein X is 5-methyl 2′-deoxycytidine; and

5′-GTXGCCCCTTCTCCCXGCAGC-3′ (SEQ ID NO: 4), wherein X is 5-methyl 2′-deoxycytidine.

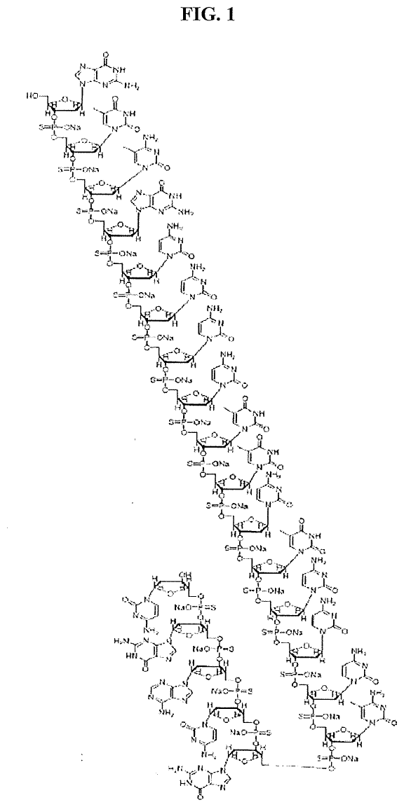

Contemplated antisense oligonucleotides include those comprising SEQ ID NO: 4: 5′-GTX GCC CCT TCT CCC XGC AGC-3′, where X represents 5-methyl-2′-deoxycytidine. In some embodiments, at least one of the internucleotide linkages of a contemplated antisense oligonucleotide is an O,O-linked phosphorothioate, for example, each of the 20 internucleotide linkages of SEQ ID NO: 4 may be an O,O-linked phosphorothioate. In a particular embodiment, the contemplated antisense oligonucleotide is an antisense oligonucleotide (referred to herein as “Mongersen”) comprising the free acid form, the salt form, or the anionic form without a counterion of SEQ ID NO: 4, wherein each of the 20 internucleotide linkages is an O,O-linked phosphorothioate linkage and shown in FIG. 1. In some embodiments, the phosphorothioate backbone of SEQ ID NO: 4 can be fully or partially protonated to form an acidic form of SEQ ID NO: 4. In some embodiments, contemplated compositions disclosed herein may include a pharmaceutically acceptable salt, e.g., a sodium salt of the antisense oligonucleotide of SEQ ID NO: 4, that optionally may include 1 to 20 O,O-linked phosphorothioate internucleotide linkages. Contemplated salts of antisense oligonucleotides include those that are fully neutralized, e.g., each phosphorothioate linkage is associated with an ion such as Na⁺. In some embodiments the contemplated salts of antisense oligonucleotides are only partially neutralized, e.g., less than all phosphorothioate linkages are associated with an ion (e.g., less than 99%, less than 95%, less than 90%, less than 85%, less than 80%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 3%, or less than 1% are neutralized). Oligonucleotides may include naturally occurring nucleobases, sugars, and covalent internucleotide (backbone) linkages as well as non-naturally occurring portions. In varying embodiments, the antisense oligonucleotides described herein, for example, the antisense oligonucleotides of SEQ ID NO: 2, SEQ ID NO: 3, and SEQ ID NO: 4, may include nucleotides comprising deoxycytidine and/or 5-methyl 2′-deoxycytidine, including, but not limited to, 5-methyl-2′-deoxycytidine 5′-monophosphate and 5-methyl-2′-deoxycytidine 5′ monophosphorothioate.

Contemplated SMAD7, IL-6, and TNFα antisense oligonucleotides include oligonucleotides that act against SMAD7, IL-6, and TNFα, respectively, and may be administered orally. Disclosed therapies may, when administered orally to a subject suffering from celiac disease, deliver an effective amount of an antisense oligonucleotide to the intestinal system of a patient, e.g., deliver an effective amount of an antisense oligonucleotide to the intestine, for example, the small intestine of a patient.

In some embodiments of the invention, the anti-SMAD7 therapy (i.e., a therapy comprising a SMAD7 antisense oligonucleotide), the anti-IL-6 therapy (i.e., a therapy comprising a IL-6 antisense oligonucleotide), or the anti-TNFα therapy (i.e., a therapy comprising a TNFα antisense oligonucleotide) may be suitable for oral delivery of an antisense oligonucleotide, e.g., tablets, that include an enteric coating, e.g., a gastro-resistant coating, such that the compositions may deliver the antisense compound to, e.g., the small intestine of a patient. For example, such administration may result in a topical effect, substantially topically applying the antisense compound directly to an affected portion of the intestine of a subject. Such administration, may, in some embodiments, substantially avoid unwanted systemic absorption of the antisense compound.

For example, a tablet for oral administration may comprise granules (e.g., is at least partially formed from granules) that include a disclosed antisense compound and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a contemplated antisense compound or a pharmaceutically acceptable salt and a pharmaceutically acceptable filler. For example, Mongersen and a filler may be blended together, with optionally other excipients, and formed into granules. In some embodiments, the intragranular phase may be formed using wet granulation, e.g., a liquid (e.g., water) is added to the blended antisense compound and filler, and then combination is dried, milled and/or sieved to produce granules. One of skill in the art would understand that other processes may be used to achieve an intragranular phase.

In some embodiments, contemplated formulations include an extra-granular phase, which may include one or more pharmaceutically acceptable excipients, and which may be blended with the intragranular phase to form a disclosed formulation.

An anti-SMAD7 therapy formulation, an anti-IL-6 therapy formulation, or an anti-TNFα therapy formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pregelatinized starch, calcium carbonate, and others including combinations thereof.

In some embodiments, an anti-SMAD7 therapy formulation, an anti-IL-6 therapy formulation, or an anti-TNFα therapy formulation may include an intragranular phase and/or an extragranular phase that includes a binder, which may generally function to hold the ingredients of the pharmaceutical formulation together. Exemplary binders include invention may be, but are not limited to, the following: starches, sugars, cellulose or modified cellulose such as hydroxypropyl cellulose, lactose, pregelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Contemplated anti-SMAD7 therapy formulations, anti-IL-6 therapy formulations, and anti-TNFα therapy formulations, e.g., that include an intragranular phase and/or an extragranular phase, may include a disintegrant such as but are not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a contemplated anti-SMAD7 therapy formulation, anti-IL-6 therapy formulation, or anti-TNFα therapy formulation includes an intra-granular phase comprising a disclosed antisense compound and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof.

In some embodiments, a contemplated anti-SMAD7 therapy formulation, anti-IL-6 therapy formulation, or anti-TNFα therapy formulation may include a lubricant, e.g., an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive tract. Enteric coatings may include a polymer that disintegrates a different rates according to pH. Enteric coatings may include for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

In some embodiments, the enteric coating includes an anionic, cationic, or neutral copolymer based on methacrylic acid, methacrylic/acrylic esters or their derivatives. In some embodiments, the enteric coating includes an ethylacrylate-methacrylic acid copolymer. Commercially available enteric coatings include Opadry® AMB, Acryl-EZE®, Eudragit® grades. In some embodiments, the enteric coating makes up about 5% to about 10%, about 5% to about 20%, about 8 to about 15%, about 8% to about 18%, about 10% to about 12%, or about 12% to about 16%, of a contemplated tablet by weight.

For example, each of an anti-SMAD7 therapy, an anti-IL-6 therapy, and an anti-TNFα therapy in the form of a tablet is provided that comprises or consists essentially of about 0.5% to about 70%, e.g., about 0.5% to about 10%, or about 1% to about 20%, by weight of an antisense oligonucleotide or a pharmaceutically acceptable salt thereof (e.g., Mongersen). Such a tablet may include for example, about 0.5% to about 60% by weight of mannitol, e.g., about 30% to about 50% by weight mannitol, e.g., about 40% by weight mannitol; and/or about 20% to about 40% by weight of microcrystalline cellulose, or about 10% to about 30% by weight of microcrystalline cellulose. For example, a contemplated tablet may comprise an intragranular phase that includes about 30% to about 60%, e.g., about 45% to about 65% by weight, or alternatively, about 5 to about 10% by weight of a SMAD7 antisense oligonucleotide (e.g., Mongersen), an IL-6 antisense oligonucleotide, or a TNFα antisense oligonucleotide, about 30% to about 50%, or alternatively, about 5% to about 15% by weight mannitol, about 5% to about 15% microcrystalline cellulose, about 0% to about 4%, or about 1% to about 7% hydroxypropylmethyl cellulose, and about 0% to about 4%, e.g., about 2% to about 4% sodium starch glycolate by weight.

Exemplary anti-SMAD7 therapy formulations, anti-IL-6 therapy formulations, or anti-TNFα therapy formulations include dosage forms that include or consist essentially of about 10 mg to about 500 mg of a SMAD7 antisense oligonucleotide (e.g., Mongersen), an IL-6 antisense oligonucleotide, or a TNFα antisense oligonucleotide, for example, tablets that include about 10 mg, about 15 mg, about 20 mg, about 25 mg, about 30 mg, about 35 mg, about 40 mg, about 50 mg, about 60 mg, about 70 mg, about 80 mg, about 90 mg, about 100 mg, about 150 mg, about 200 mg, or about 250 mg of a SMAD7 antisense oligonucleotide (e.g., Mongersen), an IL-6 antisense oligonucleotide, or a TNFα antisense oligonucleotide are contemplated herein. In one embodiment, the anti-SMAD7 therapy may be a tablet for oral use comprising: about 0.5% to about 10% by weight of an antisense oligonucleotide represented by SEQ ID NO: 4 or a pharmaceutically acceptable salt thereof; about 30% to about 50% by weight mannitol; and about 10% to about 30% by weight microcrystalline cellulose.

In an exemplary embodiment of the invention, a pharmaceutically acceptable tablet for oral administration is provided that includes an intra-granular phase that may comprise about 50% by weight of a SMAD7 antisense oligonucleotide (e.g., Mongersen, or a salt thereof), an IL-6 antisense oligonucleotide, or a TNFα antisense oligonucleotide, about 11.5% by weight mannitol, about 10% by weight microcrystalline cellulose, about 3% by weight hydroxypropylmethyl cellulose, and about 2.5% by weight sodium starch glycolate; and an extra-granular phase that may comprise about 20% by weight microcrystalline cellulose, about 2.5% by weight sodium starch glycolate, and about 0.5% by weight magnesium stearate. The tablet may also include an enteric coating.

In another exemplary embodiment, a pharmaceutically acceptable tablet for oral administration is provided that includes or consists essentially of: an intra-granular phase that may comprise or consist essentially of about 5% to about 10%, e.g., about 8% by weight of a SMAD7 antisense oligonucleotide (e.g., Mongersen, wherein the internucleotide linkages are each O,O-linked phophorothioates, and/or a salt thereof, e.g., a sodium salt), an IL-6 antisense oligonucleotide, or a TNFα antisense oligonucleotide, about 40% by weight mannitol, about 8% by weight microcrystalline cellulose, about 5% by weight hydroxypropylmethyl cellulose, and about 2% by weight sodium starch glycolate; and an extra-granular phase that may comprise about 17% by weight microcrystalline cellulose, about 2% by weight sodium starch glycolate, and about 0.4% by weight magnesium stearate.

Contemplated tablets may also include an enteric coating, e.g., a disclosed tablet may include about 13%, about 14%, about 15%, about 16%, or about 17% by weight of an enteric coating, e.g., ethylacrylate-methacrylic acid copolymers (e.g., AcrylEZE®).

For example, the anti-SMAD7 therapy, the anti-IL-6 therapy, or the anti-TNFα therapy may be in the form of a pharmaceutically acceptable tablet for oral use comprising an intra-granular phase and extra-granular phase, wherein for example, the intra-granular phase comprises about 5% to about 10%, by weight (for example about 8% by weight) of a SMAD7 antisense oligonucleotide (e.g., an antisense oligonucleotide represented by SEQ ID NO: 4 or a pharmaceutically acceptable salt thereof), an IL-6 antisense oligonucleotide, or a TNFα antisense oligonucleotide, about 40% by weight mannitol, about 8% by weight microcrystalline cellulose, about 5% by weight hydroxypropylmethyl cellulose, and about 2% by weight sodium starch glycolate, and for example, the extra-granular phase comprises about 17% by weight microcrystalline cellulose, about 2% by weight sodium starch glycolate, and about 0.4% by weight magnesium stearate, where the tablet may further comprise an enteric coating.

Contemplated formulations, e.g., tablets, in some embodiments, when orally administered to the patient may result in minimal plasma concentration of the oligonucleotide in the patient. In another embodiment, contemplated formulations, when orally administered to a patient, topically deliver to the terminal ileum and/or right colon of a patient, e.g., to an affected or diseased intestinal site of a patient. Formulations suitable for oral administration may be in the form of capsules, cachets, pills, tablets, lozenges (using, e.g., a flavored basis such as sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

### Celiac Disease

Celiac disease is a gluten-driven enteropathy affecting approximately 1% of the human population. In individuals with celiac disease, exposure to gluten is thought to stimulate expression of high levels of inflammatory cytokines, including interferon-γ, IL-21, IL-15, and IL-17. Celiac disease is also associated with high levels of IL-25 expression in the gut. Celiac disease can promote development of other diseases when left untreated, including Type I diabetes, multiple sclerosis, dermatitis herpetiformis, anemia, osteoporosis, infertility, miscarriage, epilepsy, migraines, short stature, and intestinal cancers. Celiac disease is associated with symptoms, including: abdominal bloating and pain, chronic diarrhea, vomiting, constipation, pale, foul-smelling, or fatty stool, weight loss, fatigue, irritability and behavioral issues, delayed growth and puberty, short stature, failure to thrive, and Attention Deficit Hyperactivity Disorder (ADHD).

In some embodiments of the invention, the modified Marsh classification system may be used to diagnose, treat, prevent, or manage celiac disease in a patient with celiac disease. For instance, celiac disease in a patient may be classified as type 0, type 1, type 2, type 3a, type 3b, or type 3c according to the modified Marsh classification scheme (Oberhuber et al. (1999) “The histopathology of coeliac disease: time for a standardized report scheme for pathologists” Eur J Gastroenterol Hepatol 11:1185-1194). For example, in embodiments of the invention, scoring disease state according to the Marsh classification scheme may be used to analyze the level of a TGF-β signaling activity and to determine whether a patient is a candidate for treatment with a SMAD7 antisense oligonucleotide. In some embodiments, a change in Marsh classification scheme score may be used to to determine whether a patient receiving a SMAD7 antisense oligonucleotide, a specific inhibitor of IL-6, or a specific inhibitor of TNFα should receive a higher dose or a lower dose of the SMAD7 antisense oligonucleotide, the specific inhibitor of IL-6, or the specific inhibitor of TNFα. For example, in one embodiment, the method includes administering a SMAD7 antisense oligonucleotide, a specific inhibitor of IL-6, or a specific inhibitor of TNFα to a patient with celiac disease, analyzing the patient's Marsh classification scheme score, and, if the Marsh classification scheme score is 3a, 3b, or 3c, administering a further dose of SMAD7 antisense oligonucleotide, a specific inhibitor of IL-6, or a specific inhibitor of TNFα, for example, an equal dose or a higher dose. For example, in one embodiment, the method includes administering to the patient a dose of a SMAD7 antisense oligonucleotide, a specific inhibitor of IL-6, or a specific inhibitor of TNFα to a patient with a Marsh classification scheme score of 3a, 3b, or 3c.

Refractory celiac disease is defined by persistent or recurrent malabsorptive symptoms and villous atrophy despite strict adherence to a gluten-free diet for at least 6-12 months in the absence of other causes of non-responsive treated celiac disease and overt malignancy. Refractory celiac disease affects approximately 2 to 3 times as many women as men. Patients with refractory celiac disease experience persistent diarrhea, abdominal pain, involuntary weight loss, vitamin deficiencies, anemia, fatigue, and malaise. Many patients suffering from refractory celiac disease also suffer from other autoimmune disorders. Approximately 1% of patients with Crohn's disease experience refractory celiac disease as a result of persistent overproduction of TNFα, IL-6, and IL-21. Refractory celiac disease is correlated with enhanced risk of T-cell lymphoma and a high mortality rate. For instance, within 5-6 years of diagnosis with Type II Refractory Celiac Disease (see below), half of patients will die due to development of an enteropathy-associated T cell lymphoma.

Refractory celiac disease may be further classified as Type I Refractory Celiac Disease (Type I) and Type II Refractory Celiac Disease (Type II) based on analysis of abnormal intraepithelial lymphocyte phenotype (Rubio-Tapia and Murray, (2010) “Classification and Management of Refractory Celiac Disease” Gut 59:547-557). Type I and Type II can be distinguished by the presence of abnormal clonal intraepithelial lymphocytes exclusive to Type II. In order to diagnose for the presence of abnormal clonal intraepithelial lymphocytes two criteria can be used. Abnormal lymphocytes may be classified by the loss of normal surface markers, CD3, CD4, and CD8, with preserved intracytoplasmic expression of CD3 in >50% of intraepithelial lymphocytes as determined by immunohistochemistry. Duodenal biopsies of Type II patients reveal enhanced numbers of aberrant intraepithelial lymphocytes characterized as TcRβ+, CD3−, CD4−, CD7−. The presence of clonal lymphocytes may be determined by detecting clonal rearrangements of T-cell receptor chains γ or δ via polymerase chain reaction.

In some embodiments, a patient with celiac disease may receive a combination therapy. For instance, in some embodiments a SMAD7 antisense oligonucleotide, a specific inhibitor of IL-6, and/or a specific inhibitor of TNFα is administered in combination with any of Prednisone, budesonide, azathioprine, steroids, cyclosporin, infliximab, alemtuzumab, cladribine, pentostatin, or a combination thereof, to a patient with celiac disease.

### Inflammatory Bowel Disease

“Inflammatory bowel disease” or “IBD,” as used herein, may refer to a number of chronic inflammatory diseases including Crohn's disease (CD), gastroduodenal Crohn's disease, Crohn's (granulomatous) colitis, ulcerative colitis (UC), collagenous colitis, lymphocytic colitis, ischaemic colitis, diversion colitis, Behcet's disease, microscopic colitis, ulcerative proctitis, proctosigmoiditis, jejunoileitis, left-sided colitis, pancolitis, ileocolitis, ileitis, and indeterminate colitis. CD and UC are the two most common forms of IBD. IBD is an autoimmune disease of the digestive system. CD may be localized to any portion of the gastrointestinal tract, including the terminal ileum, and may impact all cell types of the gastrointestinal tract. UC is localized to the colon and rectum, and affects cells of the mucosa only.

### Treatment and Management

A “patient,” as described herein, refers to any animal suffering from or diagnosed for celiac disease, including, but not limited to, mammals, primates, and humans. In certain embodiments, the subject may be a non-human mammal such as, for example, a cat, a dog, or a horse. In a preferred embodiment, the subject is a human subject.

The terms “treat,” “treatment,” “treating,” and the like are used herein to generally mean obtaining a desired pharmacological and/or physiological effect. The effect may be therapeutic in terms of partially or completely curing a disease and/or adverse effect attributed to the disease. The term “treatment” as used herein covers any treatment of a disease in a mammal, particularly a human, and includes: (a) inhibiting the disease, i.e., preventing the disease from increasing in severity or scope; (b) relieving the disease, i.e., causing partial or complete amelioration of the disease; or (c) preventing relapse of the disease, i.e., preventing the disease from returning to an active state following previous successful treatment of symptoms of the disease or treatment of the disease.

The terms “manage,” “management,” “managing,” and the like are used herein to generally mean controlling the severity or manifestation of symptoms of a disease, or the means of treating the disease. Generally, management is used to obtaining a desired pharmacological and/or physiological effect. The effect may be therapeutic in terms of partially or completely curing a disease and/or adverse effect attributed to the disease or ensuring that a particular symptom or manifestation of the disease does not occur or reoccur in a patient or does not rise to an undesirable or intolerable level in a patient. The term “management” as used herein covers any management of a disease in a mammal, particularly a human, and includes: (a) inhibiting the disease, i.e., preventing the disease from increasing in severity or scope; (b) relieving the disease, i.e., causing partial or complete amelioration of the disease; or (c) preventing relapse of the disease, i.e., preventing the disease from returning to an active state following previous successful treatment of symptoms of the disease or treatment of the disease. “Management” as used herein may also be used with reference to administration of a specific treatment for the disease, for example, a SMAD7 antisense oligonucleotide, a specific inhibitor of IL-6, or a specific inhibitor of TNFα.

In some embodiments of the invention, a patient having celiac disease will be administered an initial dose of an anti-SMAD7 therapy, for instance, an anti-SMAD7 oligonucleotide (e.g., a SMAD7 antisense oligonucleotide, SMAD7 RNAi, or SMAD7 miRNA), or an anti-IL-6 therapy, or an anti-TNFα therapy. As used herein, “initial dose” refers to a dose of an anti-SMAD7 therapy, an anti-IL-6 therapy, or an anti-TNFα therapy administered to a patient having celiac disease, in a series of doses. A series of doses may include one or more doses. For instance, a series of doses may comprise a single dose of an anti-SMAD7 therapy, an anti-IL-6 therapy, or an anti-TNFα therapy or more than a single dose of an anti-SMAD7 therapy, an anti-IL-6 therapy, or an anti-TNFα therapy. An initial dose may be a dose of an anti-SMAD7 therapy, an anti-IL-6 therapy, or an anti-TNFα therapy administered to a patient prior to any later dose administered to the patient. For instance, an initial dose may be, but is not limited to, the first dose of an anti-SMAD7 therapy, an anti-IL-6 therapy, or an anti-TNFα therapy administered to a treatment-naïve patient. An initial dose may also be a first dose in any treatment cycle of the anti-SMAD7 therapy, the anti-IL-6 therapy, or the anti-TNFα therapy. For example, an initial dose may be the first dose of a first treatment cycle, of a second treatment cycle, or of any subsequent treatment cycles. Alternatively, an “initial dose” may be the first dose administered to a patient after analyzing TGF-β signaling activity levels and/or another biomarker or biomarkers in a patient, or may be the most recently administered dose before a determination of the levels of TGF-β signaling activity and/or another biomarker or biomarkers in a patient.

In some embodiments of the invention, a patient having celiac disease will be administered a subsequent dose of an anti-SMAD7 therapy, for instance, a SMAD7 antisense oligonucleotide, an anti-IL-6 therapy, or an anti-TNFα therapy. As used herein, “subsequent dose” refers to a dose of an anti-SMAD7 therapy, an anti-IL-6 therapy, or an anti-TNFα therapy administered to a patient having celiac disease, after administration of a prior dose, for example, an initial dose. Thus, a subsequent dose may be administered to a patient having celiac disease in a series of doses comprising two or more doses. Furthermore, in some instances, the amount of a subsequent dose may be calibrated with respect to an initial dose or a prior dose, such that a subsequent dose is greater, equal to, or lesser than a prior dose. Calibration of the amount of a subsequent dose may be based on levels or changes in levels of TGF-β signaling activity and/or another biomarker or biomarkers in a patient having celiac disease, for instance: levels of TGF-β signaling activity in a patient having celiac disease analyzed prior to or after a prior dose, for instance, an initial dose; or changes in TGF-β signaling activity levels in a patient having celiac disease before and after a prior dose, for instance, an initial dose. A subsequent dose may be a dose administered to a patient having celiac disease after a first dose, for instance, an initial dose, of an anti-SMAD7 therapy, an anti-IL-6 therapy, or an anti-TNFα therapy administered to a patient having celiac disease. A subsequent dose may also be a dose administered after a prior dose of an anti-SMAD7 therapy, an anti-IL-6 therapy, or an anti-TNFα therapy administered to a patient having celiac disease, for instance, a dose administered after a prior dose in the same round of treatment or a different round of treatment, for instance, a previous round of treatment. A subsequent dose may be a subsequent dose with respect to any prior dose, for instance, a prior dose immediately preceding the subsequent dose or a prior dose followed by one or more doses administered prior to administration of the subsequent dose.

Patients treated using an above method may or may not have detectable fibrosis. In some embodiments, the patient has at least about a 5%, 10%, 20%, 30%, 40% or even 50% or more reduction in the amount of fibrosis present in the patient after administering a specific inhibitor of SMAD7, a specific inhibitor of IL-6, or a specific inhibitor of TNFα, after e.g., 1 day, 2 days, 1 week, 1 month, or 6 months, or more. Administering an inhibitor of SMAD7, a specific inhibitor of IL-6, or a specific inhibitor of TNFα may be on, e.g., at least a daily basis. The delay of clinical manifestation of fibrosis in a patient as a consequence of administering an inhibitor of SMAD7, a specific inhibitor of IL-6, or a specific inhibitor of TNFα may be at least e.g., 6 months, 1 year, 18 months or even 2 years or more as compared to a patient who is not administered an inhibitor of SMAD7.

A patient at risk of celiac disease may include those patients with ulcerative colitis, inflammatory bowel disease, or Crohn's disease. A patient at risk may also include those patients with an early age at diagnosis of Crohn's or colitis, extensive and/or severe colonic disease, patients with dermatitis herpetiformis, Type 1 diabetes, Down syndrome, or Turner syndrome, autoimmune thyroid disease, Sjogren's syndrome, or microscopic colitis (i.e., lymphocytic or collagenous colitis).

As used herein, “anti-SMAD7 oligonucleotide” refers to an oligonucleotide comprising a nucleic acid sequence that is complementary to a nucleic acid sequence in the coding or noncoding region of SMAD7. In some embodiments, the anti-SMAD7 oligonucleotde comprises a SMAD7 antisense oligonucleotide, SMAD7 RNAi, or SMAD7 miRNA. In some embodiments, the anti-SMAD7 oligonucleotide can reduce the expression of a gene that comprises a complementary nucleic acid sequence when the anti-SMAD7 oligonucleotide is introduced into a cell (e.g., an immune cell, such as PBMC, pDC, or B-cell). In some embodiments, the anti-SMAD7 oligonucleotide can reduce expression of an mRNA transcribed from the gene. In some embodiments, the anti-SMAD7 oligonucleotide can reduce expression of a protein encoded by the gene. In some embodiments, the anti-SMAD7 oligonucleotide can reduce secretion of a protein encoded by the gene from the cell into which the anti-SMAD7 oligonucleotide was introduced.

As used herein, a “SMAD7 antisense oligonucleotide” is an oligonucleotide comprising a nucleic acid sequence that is complementary to the nucleic acid sequence of a SMAD7 mRNA, SMAD7 cDNA, or the coding strand of a SMAD7 DNA.

As used herein, “SMAD7” (also known as CRCS3, FLJ16482, MADH7, MADH8, MAD (mothers against decapentaplegic, Drosophila) homolog 7, MAD homolog 8, SMAD, mothers against DPP homolog 7, mothers against DPP homolog 8) means the human protein or any of the mRNA transcripts encoded by the gene identified by Entrez GeneID No. 4092 and allelic variants thereof.

As used herein, “TGF-β1” (also known as CED; LAP; DPD1; TGFB; and TGFbeta) means the human protein or any of the mRNA transcripts encoded by the gene identified by Entrez GeneID No. 7040 and allelic variants thereof.

As used herein, “TGF-β2” (also known as LDS4 and TGF-beta2) means the human protein or any of the mRNA transcripts encoded by the gene identified by Entrez GeneID No. 7042 and allelic variants thereof.

As used herein, “TGF-β3” (also known as ARVD; LDS5; RNHF; ARVD1; and TGF-beta3) means the human protein or any of the mRNA transcripts encoded by the gene identified by Entrez GeneID No. 7043 and allelic variants thereof.

As used herein, “SMAD2” (also known as JV18; MADH2; MADR2; JV18-1; hMAD-2; and hSMAD2) means the human protein or any of the mRNA transcripts encoded by the gene identified by Entrez GeneID No. 4087 and allelic variants thereof.

As used herein, “SMAD3” (also known as LDS3; LDS1C; MADH3; JV15-2; HSPC193; and HsT17436) means the human protein or any of the mRNA transcripts encoded by the gene identified by Entrez GeneID No. 4088 and allelic variants thereof.

As used herein, “SMAD4” (also known as JIP; DPC4; MADH4; and MYHRS) means the human protein or any of the mRNA transcripts encoded by the gene identified by Entrez GeneID No. 4089 and allelic variants thereof.

As used herein, “TNFα” (also known as DIF; TNFA; TNFSF2; TNLG1F; and TNF-alpha) means the human protein or any of the mRNA transcripts encoded by the gene identified by Entrez GeneID No. 7124 and allelic variants thereof.

As used herein, “IL-6” (also known as CDF; HGF; HSF; BSF2; IL-6; BSF-2; IFNB2; and IFN-beta-2) means the human protein or any of the mRNA transcripts encoded by the gene identified by Entrez GeneID No. 3569 and allelic variants thereof.

As used herein, “IL-25” (also known as IL17E) means the human protein or any of the mRNA transcripts encoded by the gene identified by Entrez GeneID No. 64806 and allelic variants thereof.

As used herein, “IL-15” (also known as Interleukin 15) means the human protein or any of the mRNA transcripts encoded by the gene identified by Entrez GeneID No. 3600 and allelic variants thereof.

Methods of Monitoring Treatment

In some embodiments, the methods described herein entail monitoring the treatment, disease state, or biomarkers associated with a disease state of a patient having celiac disease. Monitoring treatment may be useful in terms of assessing treatment efficacy and safety, as well as evaluating the need to modulate treatment. Monitoring treatment may also be useful for evaluating whether the amount of SMAD7 antisense oligonucleotide, specific inhibitor of IL-6, or specific inhibitor of TNFα being administered to a patient or which will be administered to a patient should be increased or decreased. Furthermore, monitoring treatment may be useful in terms of determining the amount or relative amount by which a dose of SMAD7 antisense oligonucleotide, specific inhibitor of IL-6, or specific inhibitor of TNFα should be modulated, i.e., increased or decreased.

Monitoring, for example, monitoring of TGF-β signaling activity levels in a patient having celiac disease, may commence prior to, during, or after an initial dose of a SMAD7 antisense oligonucleotide a specific inhibitor of IL-6, or a specific inhibitor of TNFα. Furthermore, monitoring may continue after an initial dose. For example monitoring may be performed after administration of an initial dose. Monitoring may also be performed before, during, or after a subsequent dose of SMAD7 antisense oligonucleotide, specific inhibitor of IL-6, or specific inhibitor of TNFα. Monitoring may be continuous or discontinuous such that monitoring may be performed at regular intervals, for example, after each dose of a SMAD7 antisense oligonucleotide, specific inhibitor of IL-6, or specific inhibitor of TNFα is administered to a patient, before each dose of a SMAD7 antisense oligonucleotide, a specific inhibitor of IL-6, or a specific inhibitor of TNFα is administered to a patient, or before and after each dose of a SMAD7 antisense oligonucleotide, a specific inhibitor of IL-6, or a specific inhibitor of TNFα is administered to a patient. Monitoring may be performed multiple times in a single day (for instance, 2 times, 3 times, 4 times, about five times, or about 10 times in a single day), once a day, multiple times in a single week (for instance, 2 times, 3 times, 4 times, about five times, or about 10 times in a single week), once a week, multiple times in a single month (for instance, 2 times, 3 times, 4 times, about five times, or about 10 times in a single month), or once a month. In methods of the invention, monitoring may be performed at various times relative to an administering step. For instance, in some embodiments, monitoring may be performed immediately after, or at least 1 day, at least 3 days, at least 5 days, at least 1 week, at least 2 weeks, at least 3 weeks, at least 1 month, at least 2 months, at least 4 months, or at least 6 months after an administration step. In some embodiments, monitoring is performed about 15 days or about 28 days after an administration step.

As described above, the invention is based in part on the discovery that levels of TGF-β signaling activity can be used to evaluate and modify management and treatment with a SMAD7 antisense oligonucleotide, a specific inhibitor of IL-6, or a specific inhibitor of TNFα in a patient having celiac disease. Thus, in embodiments of the invention, it is useful to know, determine, analyze, or compare levels of TGF-β signaling activity in a patient or a sample from a patient having celiac disease. For example, in some instances it will be useful to know a threshold value for normal or abnormal levels of TGF-β signaling activity in order to determine whether levels of the SMAD7 antisense oligonucleotide, the specific inhibitor of IL-6, or the specific inhibitor of TNFα should be increased, decreased, or left untouched. In the methods described herein, a normal level of TGF-β signaling activity may be tied to a specific value for a concentration of an analyte, for example, TGF-β1 or p-SMAD3, in a patient sample such as a tissue, cellular, or serum sample, for instance, a value of 0.01 pg/ml, about 0.1 pg/ml, about 1 pg/ml, about 2 pg/ml, about 3 pg/ml, about 4 pg/ml, about 5 pg/ml, about 6 pg/ml, about 7 pg/ml, about 8 pg/ml, about 9 pg/ml, about 10 pg/ml, about 11 pg/ml, about 12 pg/ml, about 13 pg/ml, about 14 pg/ml, about 15 pg/ml, about 17.5 pg/ml, about 20 pg/ml, about 22.5 pg/ml, about 25 pg/ml, about 30 pg/ml, or about 35 pg/ml, about 12.5 pg/ml, about 16 pg/ml, about 17 pg/ml, about 18 pg/ml, about 19 pg/ml, about 40 pg/ml, about 50 pg/ml, about 60 pg/ml, about 70 pg/ml, about 80 pg/ml, about 90 pg/ml, or about 100 pg/ml. In some embodiments, a normal level of TGF-β signaling activity, for example, TGF-β1 or p-SMAD3, in a sample, for example may be determined by comparison to median levels of TGF-β signaling activity in a healthy control group that is matched to the patient with respect to various factors, for example, age, gender, ethnic origin, smoking habits, dietary habits, body-mass index (BMI), and/or exercise habits.

Levels of TGF-β signaling activity may be determined by obtaining a sample from the patient. According to the methods described herein, a sample may be a tissue sample (e.g., an intestinal tissue sample, a duodenal tissue sample, or a jejunal tissue sample) or a bodily fluid sample (e.g., a saliva sample, a stool, or a urine sample). A sample can be a sample obtained from a patient tissue biopsy, for example, a mucosal tissue biopsy, for example, an intestinal mucosal tissue biopsy, for example a small intestinal mucosal tissue biopsy. Furthermore, the sample may be a blood, serum, or plasma sample. A blood sample from a subject may be obtained using techniques well-known in the art. Blood samples may include peripheral blood mononuclear cells (PMBCs), RBC-depleted whole blood, or blood serum. PBMCs can be separated from whole blood samples using different density gradient (e.g., Ficoll density gradient) centrifugation procedures. For example, whole blood (e.g., anticoagulated whole blood) is layered over the separating medium and centrifuged. At the end of the centrifugation step, the following layers are visually observed from top to bottom: plasma/platelets, PBMC, separating medium and erythrocytes/granulocytes.

Methods of monitoring treatment may also include methods of monitoring factors, including, but not limited to levels of analytes (e.g., TGF-β1, TGF-β2, TGF-β3, SMAD2, SMAD3, SMAD4, p-SMAD2, p-SMAD3, TNFα, IL-6, IL-25, SMAD7, and/or IL-15) and the presence or severity of celiac disease symptoms.

In embodiments of the invention where levels of an analyte (e.g., TGF-β1, TGF-β2, TGF-β3, SMAD2, SMAD3, SMAD4, p-SMAD2, p-SMAD3, TNFα, IL-6, IL-25, SMAD7, or IL-15) are measured, various methods may be used to measure the analyte. For example, the level of an analyte, for example, TGF-β1, TGF-β2, TGF-β3, SMAD2, SMAD3, SMAD4, p-SMAD2, p-SMAD3, TNFα, IL-6, IL-25, SMAD7, or IL-15, may be determined by immunochemistry and/or by nucleotide analysis. Methods of determining analyte concentration by immunochemistry include, but are not limited to, Western blotting, ELISA, and immunostaining methods. In some embodiments, a method of determining analyte concentration by immunochemistry is performed using an antibody that can bind to the analyte of interest, for instance, an anti-p-SMAD3 antibody or an anti-TGF-β1 antibody. Methods of determining analyte concentration by immunochemistry may also involve the use of buffers, blocking reagents, unconjugated primary antibodies, and primary and/or secondary antibodies conjugated to tags that allow for antibody detection, such as fluorescent probes or substrate-specific enzymes.

Methods of determining analyte concentration by nucleotide analysis include, but are not limited to, methods of analyzing analyte mRNA transcript levels such as Northern blotting and polymerase chain reaction methods, for example, quantitative polymerase chain reaction methods. Nucleotide analysis may be performed using an oligonucleotide probe that binds an analyte nucleotide sequence (e.g., a TGF-β1, TGF-β2, TGF-β3, SMAD2, SMAD3, SMAD4, TNFα, IL-6, IL-25, SMAD7, or IL-15 nucleotide sequence) or a pair of oligonucleotide primers capable of amplifying an analyte nucleotide sequence via a polymerase chain reaction, for example, by a quantitative polymerase chain reaction. Oligonucleotide probes and oligonucleotide primers may be linked to a detectable tag, such as, for example, a fluorescent tag. In determining analyte concentration by nucleotide analysis, the practitioner may evaluate a particular analyte's mRNA transcript concentration in a sample. Alternatively, in determining analyte concentration by nucleotide analysis, the practitioner may establish a correlation between a particular analyte's mRNA transcript abundance and the particular analyte's protein abundance in order to extrapolate analyte protein concentration based on a measure of analyte mRNA transcript abundance.

Methods of the claimed invention include steps that may be carried out in vitro. For instance, it is contemplated that the steps of measuring TGF-β signaling activity levels in the subject may involve determining the levels of TGF-β signaling activity in a sample. For example, the level of TGF-β signaling activity in a sample may be determined by performing immunochemistry or nucleotide analysis on the sample in vitro. Alternatively, in some embodiments of the invention, the steps of determining and analyzing the TGF-β signaling activity levels in a patient having celiac disease or determining and analyzing the TGF-β signaling activity levels in a sample may be carried out in vivo.

### Enzyme-Linked Immunosorbent Assay

In some embodiments, TGF-β signaling activity as determined by analyte concentration may be measured by Enzyme-linked immunosorbent assay (ELISA). Specifically, levels of TGF-β signaling activity as determined by measuring analyte concentration in a sample, especially a blood sample, for example, a blood serum sample, can be determined by ELISA. Assaying analyte concentration by ELISA requires at least one antibody against the analyte protein, e.g., at least one anti-p-SMAD3 antibody or anti-TGF-β1 antibody and/or at least one secondary antibody, e.g., at least one labeled secondary antibody. In some embodiments, the primary antibody is labeled with, e.g., a fluorescent label. In certain embodiments, the primary antibody is not labeled and a secondary antibody capable of binding the species isotype of the primary antibody is labeled, e.g., with a fluorescent probe or enzyme capable of reacting with a specific substrate, thereby providing a detectable signal.

Performing an ELISA requires at least one capture antibody, at least one detection antibody, and/or at least one enzyme-linked or fluorescent labeled secondary antibody. For example, assaying TGF-β signaling activity levels by ELISA may require an anti-p-SMAD3 antibody or anti-TGF-β1 antibody, respectively, as the capture antibody. The antibody is immobilized on a solid support such as a polystyrene microtiter plate. A sample, for example, a blood serum sample is then added and allowed to complex with the bound antibody. Unbound serum components are removed with a wash. A detection antibody, e.g., a different anti-p-SMAD3 antibody or anti-TGF-β1 antibody, e.g., an anti-p-SMAD3 antibody or anti-TGF-β1 antibody, that binds to a different portion of the p-SMAD3 or TGF-β1 protein, respectively, than the capture antibody, is added and is allowed to bind to the captured p-SMAD3 or TGF-β1. The detection antibody is linked to a detectable tag, such as an enzyme, either directly or indirectly, e.g., through a secondary antibody that specifically recognizes the detection antibody. Typically between each step, the plate, with bound protein, is washed with a wash buffer, e.g., a mild detergent solution. Typical ELISA protocols also include one or more blocking steps, which involve use of a non-specifically-binding protein such as bovine serum albumin to block unwanted non-specific binding of protein reagents to the plate. After a final wash step, the plate is developed by addition of an appropriate enzyme substrate, to produce a visible signal, which indicates the amount of p-SMAD3 or TGF-β1 protein in the sample. The substrate can be, e.g., a chromogenic substrate or a fluorogenic substrate. ELISA methods, reagents and equipment are well-known in the art and commercially available.

### Nucleotide Analysis

In some embodiments, levels of TGF-β signaling activity as determined by measuring levels of analytes may be determined by performing a “nucleotide analysis.” A nucleotide analysis may include analysis of analyte nucleotide transcript levels (e.g., SMAD3 or TGF-β1 mRNA transcript levels) in a sample, for example, a blood sample. Analyte transcript levels may be determined by Northern blot, for example, a quantitative Northern blot; or polymerase chain reaction, for example, a quantitative polymerase chain reaction. Reagents necessary to perform Northern blot include oligonucleotide probes, for example, oligonucleotide probes linked to a detectable label. Detectable labels may include fluorescent labels or enzymes capable of reacting with a specific substrate. Reagents necessary to perform polymerase chain reaction include oligonucleotide primers capable of specifically binding to a particular analyte mRNA transcript and amplifying the number of analyte mRNA transcripts by polymerase chain reaction. Oligonucleotide primers may be linked to a detectable label to enable, for example, quantitative polymerase chain reaction. Other reagents necessary to perform quantitative polymerase chain reaction include, but are not limited to, primers capable of amplifying a control transcript signal, for instance, a beta tubulin transcript signal. Buffers, reagents (including oligonucleotide primers and probes), techniques, and equipment necessary for performing Northern blotting and polymerase chain reactions are readily available and are well-known in the art.

### Methods of Selecting Patients

The invention described herein provides methods of treating patients in part by selecting patients that show some likelihood of responsiveness to SMAD7-antisense therapy, anti-IL-6 therapy, or anti-TNFα therapy. Likelihood of responsiveness to anti-SMAD7 therapy, anti-IL-6 therapy, or anti-TNFα therapy is premised in part on determining levels of TGF-β signaling activity in a patient with celiac disease, for example, preexisting levels of TGF-β signaling activity (e.g., levels of p-SMAD3 or TGF-β1 in a patient prior to administration of an initial dose of a SMAD7 antisense oligonucleotide, a specific inhibitor of IL-6, or a specific inhibitor of TNFα) or levels of p-SMAD3 or TGF-β1 determined after an initial dose or one or more subsequent doses of SMAD7 antisense oligonucleotide, a specific inhibitor of IL-6, or a specific inhibitor of TNFα. For instance, in some embodiments of the invention, a patient will be selected for treatment or further treatment with a SMAD7 antisense oligonucleotide, a specific inhibitor of IL-6, or a specific inhibitor of TNFα after detecting or analyzing absolute or relative TGF-β signaling activity levels or changes in TGF-β signaling activity levels. Levels of TGF-β signaling activity in a patient with celiac disease may be compared to a normal level of TGF-β signaling activity, for example, normal levels of TGF-β signaling activity as defined by median TGF-β signaling activity levels in a matched control group or absolute levels of TGF-β signaling activity.

In some embodiments, a patient will be selected for treatment or further treatment with a SMAD7 antisense oligonucleotide, a specific inhibitor of IL-6, or a specific inhibitor of TNFα if the levels of TGF-β signaling activity in the patient are more than 10%, 20%, 30%, 40%, 50%, 60%, 70%, 80%, 90%, or 100% depressed relative to the average, median or mean levels of TGF-β signaling activity in a matched control group.

In some embodiments, a patient will be selected for treatment or further treatment with a SMAD7 antisense oligonucleotide, a specific inhibitor of IL-6, or a specific inhibitor of TNFα if the level of TGF-β signaling activity in the patient is more than 2-fold, more than 3-fold, more than 4-fold, more than 5-fold, more than 6-fold, more than 7-fold, more than 8-fold, more than 9-fold or more than 10-fold depressed relative to the average, median or mean levels of TGF-β signaling activity in a matched control group.

In some instances, TGF-β signaling activity levels will be measured in terms of a concentration, for instance, mass of p-SMAD3 or TGF-β1 protein, peptide, or RNA per volume of sample, for example, volume of blood or tissue. Thus selection of patients for initial or continued treatment is tied to TGF-β signaling activity levels in the patient, such that, for example, low initial levels of TGF-β signaling activity may indicate a potential for responsiveness to SMAD7 antisense oligonucleotide treatment, specific inhibitor of IL-6 treatment, or specific inhibitor of TNFα treatment. Furthermore, low levels of TGF-β signaling activity (i.e., below normal levels of TGF-β signaling activity) may indicate a need for increased doses of SMAD7 antisense oligonucleotide, specific inhibitor of IL-6, or specific inhibitor of TNFα whereas normal or above normal levels of TGF-β signaling activity may indicate a need for decreased or unchanged doses of SMAD7 antisense oligonucleotide, specific inhibitor of IL-6, or specific inhibitor of TNFα, especially following one or more doses. Alternatively, continued below normal levels of TGF-β signaling activity after repeated doses may indicate that the patient is not responsive to treatment.

Thus, if levels of TGF-β signaling activity are below normal levels of TGF-β signaling activity, a patient may be administered an initial and/or subsequent dose of SMAD7 antisense oligonucleotide, specific inhibitor of IL-6, or specific inhibitor of TNFα. In some embodiments, TGF-β signaling activity levels are already known to be below normal TGF-β signaling activity levels prior to administration of an initial dose. In some embodiments, TGF-β signaling activity levels in a patient with celiac disease will be determined prior to administration of an initial dose. In some embodiments, after an initial dose of SMAD7 antisense oligonucleotide, a specific inhibitor of IL-6, or a specific inhibitor of TNFα, if TGF-β signaling activity levels are analyzed and determined to be below normal levels of TGF-β signaling activity, the patient will be administered a subsequent dose of SMAD7 antisense oligonucleotide, a specific inhibitor of IL-6, or a specific inhibitor of TNFα, respectively, for instance a greater dose than the initial dose or a dose equal to the initial dose. Alternatively, if TGF-β signaling activity levels are analyzed and determined to be above normal levels of TGF-β signaling activity, the patient may be administered a subsequent dose of SMAD7 antisense oligonucleotide, specific inhibitor of IL-6, or specific inhibitor of TNFα, for instance an equal or smaller dose than the initial dose.

In yet other embodiments, TGF-β signaling activity levels may be analyzed and determined in a patient with celiac disease, and then an initial dose of SMAD7 antisense oligonucleotide, specific inhibitor of IL-6, or specific inhibitor of TNFα may be administered to the patient if the TGF-β signaling activity levels are below normal levels of TGF-β signaling activity. Furthermore, in some embodiments, after an initial dose of SMAD7 antisense oligonucleotide, specific inhibitor of IL-6, or specific inhibitor of TNFα, levels of TGF-β signaling activity may be determined, and if the levels of TGF-β signaling activity are below normal levels of TGF-β signaling activity then a subsequent dose of, respectively, SMAD7 antisense oligonucleotide, specific inhibitor of IL-6, or specific inhibitor of TNFα that is greater than or equal to the initial dose may be administered to the patient. Alternatively, after an initial dose of SMAD7 antisense oligonucleotide, specific inhibitor of IL-6, or specific inhibitor of TNFα, levels of TGF-β signaling activity may be determined, and if the levels of TGF-β signaling activity are above normal levels of TGF-β signaling activity then a subsequent dose of, respectively, SMAD7 antisense oligonucleotide, specific inhibitor of IL-6, or specific inhibitor of TNFα that is smaller than or equal to the initial dose may be administered to the patient.

In yet other embodiments, the invention provides methods whereby: TGF-β signaling activity levels may be analyzed and determined in a patient with celiac disease; an initial dose of SMAD7 antisense oligonucleotide, specific inhibitor of IL-6, or specific inhibitor of TNFα may be administered to the patient if the TGF-β signaling activity levels are below normal levels of TGF-β signaling activity; the levels of TGF-β signaling activity are analyzed after the initial administration; and if the level of TGF-β signaling activity after the initial dose is administered is greater than the level of TGF-β signaling activity before the initial dose is administered, then the patient is administered a subsequent dose that is the same as the initial dose or smaller than the initial dose or treatment is terminated. Alternatively, if the level of TGF-β signaling activity is unchanged or decreased after the initial dose is administered compared to the level of TGF-β signaling activity before the initial dose is administered, then the patient is administered a subsequent dose that is the same as the initial dose or greater than the initial dose.

Thus, the contemplated invention provides different methods for treating and managing celiac disease in a patient by accounting for multiple treatment scenarios based on analysis and determination of TGF-β signaling activity levels and patient responsiveness to SMAD7 antisense oligonucleotide, specific inhibitor of IL-6, or specific inhibitor of TNFα administration.

For instance, if after administration of a SMAD7 antisense oligonucleotide, a specific inhibitor of IL-6, or a specific inhibitor of TNFα, TGF-β signaling activity levels in a patient are below normal TGF-β signaling activity levels, treatment can continue at the same dose or at an increased dose of the SMAD7 antisense oligonucleotide, specific inhibitor of IL-6, or specific inhibitor of TNFα.

If after administration of a SMAD7 antisense oligonucleotide, a specific inhibitor of IL-6, or a specific inhibitor of TNFα TGF-β signaling activity levels in a patient are above normal TGF-β signaling activity levels, treatment can continue at the same dose or at a decreased dose of the SMAD7 antisense oligonucleotide, specific inhibitor of IL-6, or specific inhibitor of TNFα.

If after an initial dose and one or more subsequent doses of a SMAD7 antisense oligonucleotide, specific inhibitor of IL-6, or specific inhibitor of TNFα TGF-β signaling activity levels continue to be below normal TGF-β signaling activity levels, the treatment may be terminated. For example, treatment may be terminated because the patient is not responsive to treatment or the patient has been administered the maximum tolerated dose.

In some instances, if TGF-β signaling activity levels increase in a patient following one or more doses of the SMAD7 antisense oligonucleotide, the specific inhibitor of IL-6, or the specific inhibitor of TNFα, this may indicate that a patient is responsive to treatment. In these patients, subsequent doses of the SMAD7 antisense oligonucleotide, the specific inhibitor of IL-6, or the specific inhibitor of TNFα may be administered but at the same dose or a smaller dose compared to the previous dose(s).

In some instances, if TGF-β signaling activity levels are stable or decrease following an initial or one or more subsequent doses of the SMAD7 antisense oligonucleotide, the specific inhibitor of IL-6, or the specific inhibitor of TNFα, this may indicate that a patient is not responsive to treatment. In these patients, subsequent doses of the SMAD7 antisense oligonucleotide, the specific inhibitor of IL-6, or the specific inhibitor of TNFα may be administered but at a greater dose compared to the previous dose(s). Alternatively, the treatment can be discontinued, for example, if the dose approaches the maximum tolerated dose.

### EXAMPLES

The invention is further illustrated by the following example. The example is provided for illustrative purposes only, and is not to be construed as limiting the scope or content of the invention in any way.

### Example 1: Patient Study Population

43 patients undergoing upper endoscopy at the Gastrointestinal Unit of Tor Vergata University Hospital (Rome, Italy) or S. Matteo Hospital, University of Pavia, were enrolled in a study directed to the analysis of TGF-β signaling pathway component expression in refractory celiac disease.

The study population included 12 patients on a gluten-containing diet diagnosed as active Crohn's disease (“CD”) patients. These patients expressed serum anti-endomysium IgA antibody (EMA) and anti-tissue transglutaminase-2 (TG-2) antibody and displayed villous atrophy on histological examination according to Marsh classification (grade 3 B-C).

The study population also included 10 patients who were asymptomatic, negative for both EMA and anti-TG-2 antibodies, and had normal duodenal histology while receiving a strict gluten-free diet, but who were previously diagnosed with CD. These patients were deemed inactive CD patients (ICD) for purposes of the study.

The study population included 11 patients who were negative for both EMA and anti-TG-2 antibodies while receiving a gluten-containing diet and who displayed no villous atrophy. These patients were deemed disease-free control patients for purposes of the study.

The study population included 10 patients previously diagnosed with CD, who suffered from persistent malabsorption symptoms and displayed histological evidence of villous atrophy (Marsh, grade 3 A-C) despite a long-term gluten-free diet. This group of patients was negative for both EMA and anti-TG-2 antibodies, and a complete diagnostic work-up for other disorders was negative. These 10 patients were diagnosed with RCD. 5 of these patients were classified as suffering from RCDI, and 4 of the RCDI patients received steroid treatment while the single remaining patient did not receive drug treatment. The other 5 RCD patients were classified as suffering from RCDII. 3 of the RCDII patients had ulcerative jejunoileitis and received steroid treatment, while the remaining 2 patients received no drug treatment.

Duodenal biopsies were taken from the distal duodenum of each patient. Biopsies were either fixed in formalin and then embedded in paraffin or prepared for RNA and protein extraction.

### Example 2: SMAD7 Protein Expression is Increased in Refractory Celiac Disease

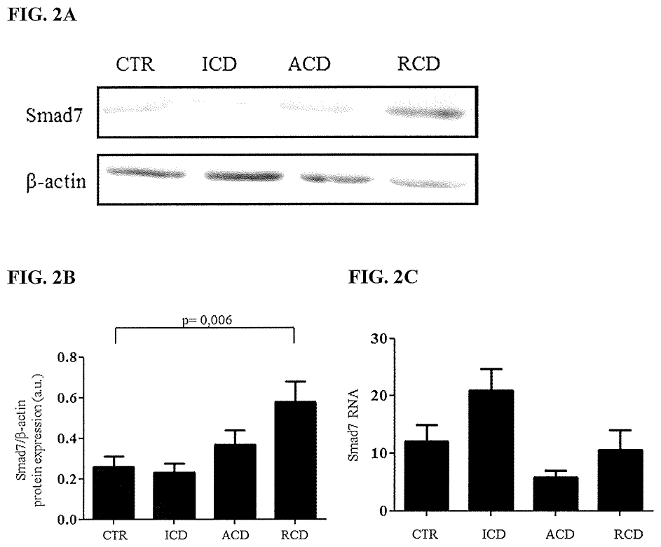

SMAD7 protein and mRNA expression in duodena of patients with refractory celiac disease were analyzed in order to determine whether changes in SMAD7 expression are associated with refractory celiac disease. Total protein extracts were collected from duodenal biopsy tissue samples of 10 disease-free patients (CTR), 10 patients with inactive celiac disease (ICD), 10 patients with active celiac disease (ACD), and 10 patients with refractory celiac disease (RCD; 5 RCDI patients and 5 RCDII patients), and probed for SMAD7 expression by Western blot (RCD; FIG. 2A). Briefly, biopsied tissue samples were lysed on ice with a buffer containing 10 mM HEPES (ph 7.9), 10 mM KCl, 0.1 mM EDTA and 0.5% Nonidet p40, supplemented with 1 mM dithiothreitol, 10 mg/ml aprotinin, 10 mg/ml leupeptin, 1 mM phenyl-methylsulfonyl fluoride, 1 mM Na3VO4 and 1 mM NaF. Lysates were clarified by centrifugation at 12,000 g for 30 min at 4° C., separated on a 10% sodium dodecyl sulphate (SDS)-polyacrylamide gel by electrophoresis, and transferred to blots. Smad7 was detected using a monoclonal mouse anti-human antibody (1 μg/ml, R&D Systems) followed by a horseradish peroxidase-conjugated rabbit anti-mouse IgG monoclonal antibody (Dako, Milan, Italy). After probing for Smad7 expression, blots were stripped and incubated with a mouse anti-human β-actin antibody followed by a rabbit anti-mouse antibody conjugated to horseradish peroxidase. The relative Smad7 signal level appeared stronger in the RCD patient samples (representative example shown in FIG. 2A). Quantification of SMAD7 protein levels by densitometric analysis in each group of patients relative to β-actin protein levels demonstrated that duodenal tissue of RCD patients expressed significantly higher levels of SMAD7 in comparison to disease-free control patient samples (p=0.006, CTR v. RCD; FIG. 2B).

Levels of SMAD7 mRNA were also analyzed in total RNA extracts prepared from duodenal tissue biopsies of 11 CTR, 9 ICD, 11 ACD, and 7 RCD (4 RCDI and 3 RCDII) patients. Total RNA was extracted using a Pure Link mRNA mini kit according to the manufacturer's instructions (Life Technologies, Milan, Italy). 1 μg/sample of RNA was retro-transcribed into complementary DNA (cDNA), and then 1 μl of cDNA/sample was amplified using a Sybergreen mastermix (Biorad, Milan, Italy). PCR was performed using the following conditions: denaturation 1 minute at 95° C.; annealing 30 seconds at 62° C. for Smad7 and at 60° C. for β-Actin. The following primer sequences were used: Smad7 forward, 5′-CGGATCTCAGGCATTCCTCG-3′; Smad7 reverse, 5′-GGCACAGCATCTGGACAGTC-3′; β-actin forward, 5′-AAGATGACCCAGATCATGTTTGAGACC-3′, and β-actin reverse, 5′-AGCCAGTCCAGACGCAGGAT-3′ was used as internal control. SMAD7 mRNA levels were not significantly different in any of the groups analyzed (FIG. 2C). These results demonstrate that while levels of SMAD7 protein in duodenal tissue of individuals with refractory celiac disease were significantly elevated relative to levels of SMAD7 protein in duodenal tissue of active celiac disease patients, inactive celiac disease patients, and healthy control patients, SMAD7 mRNA levels were not significantly different between these populations.

SMAD7 immunostaining of duodenal tissue also revealed relatively high levels of SMAD7 expression in RCD patients. Paraffin-embedded duodenal sections of 4 ACD patients, 4 ICD patients, 7 RCD patients (4 RCDI patients and 3 RCDII patients), and 4 CTR patients were analyzed for Smad7 using anti-human Smad7 mouse monoclonal antibody (R&D Systems, Minneapolis, Minn., USA, 1:50 final dilution). Isotype control IgG-stained sections were prepared under identical conditions, but were probed with a purified mouse normal IgG control antibody (R&D systems, Minneapolis, Minn., USA) rather than a Smad7 antibody.

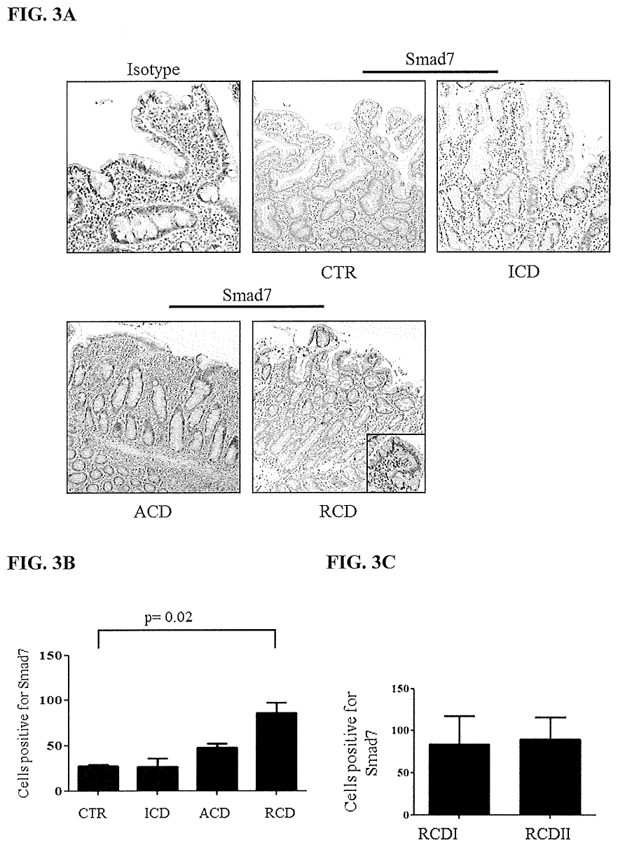

As shown in FIG. 3A, SMAD7 immunostaining intensity of lamina propria and epithelial gastrointestinal tissue appeared higher in tissue samples from RCD patients compared to ICD, ACD, and CTR patients. Isotype control staining of tissue revealed no appreciable signal. Furthermore, quantification of SMAD7-positive cells in samples from CTR, ICD, ACD, and RCD patients revealed a significantly higher number of SMAD7-positive cells in RCD tissue samples compared to CTR tissue samples (FIG. 3B, p=0.02, CTR v. RCD). No significant difference in the number of SMAD7-positive cells between RCDI and RCDII patient tissue samples was found (FIG. 3C). These results also demonstrate that SMAD7 protein expression was elevated in RCDI and RCDII patients relative to healthy control patients and patients with ICD and ACD, and that the number of cells expressing detectable SMAD7 was significantly elevated in gastrointestinal tissue from RCD patients relative to healthy control patients and patients with ICD and ACD.

### Example 3: TGF-β1 Smad-Mediated Signaling is Impaired in Refractory Celiac Disease

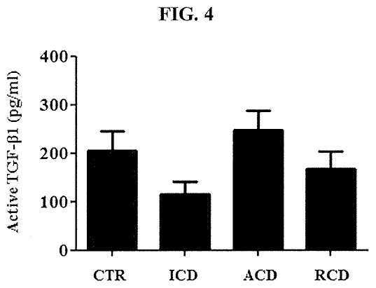

Expression of TGF-β1 and the Smad2/3 signaling components of the TGF-β pathway in healthy control patients and patients with celiac disease was also investigated. Quantification by ELISA of active TGF-β1 (i.e., cleaved, mature TGF-β1) protein levels in total protein extracts of duodenal biopsy tissue from 11 CTR, 8 ICD, 12 ACD, and 8 RCD (5 RCDI and 3 RCDII) patients revealed no significant difference between levels of active TGF-β1 between CTR and RCD samples (FIG. 4). These results demonstrate that levels of active TGF-β 1 protein in gastrointestinal tissue of patients with refractory celiac disease were not significantly lower than those of healthy control patients.

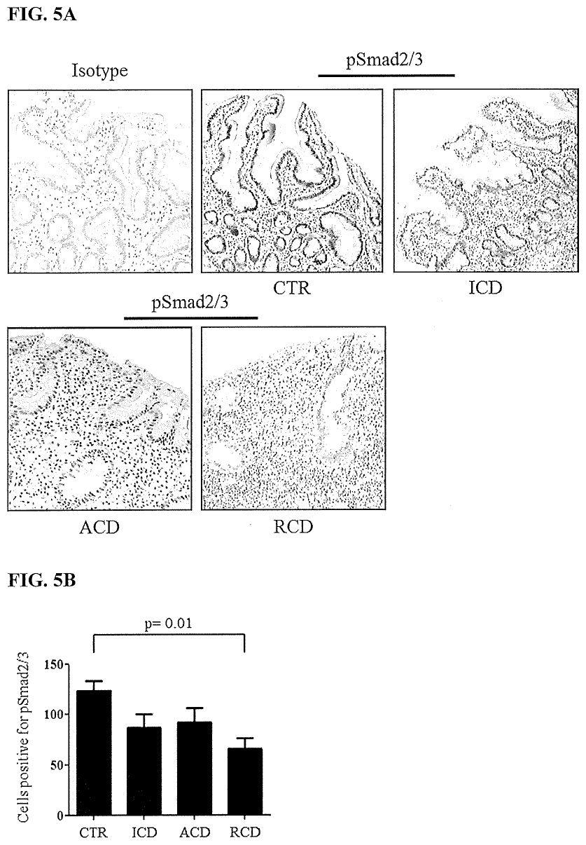

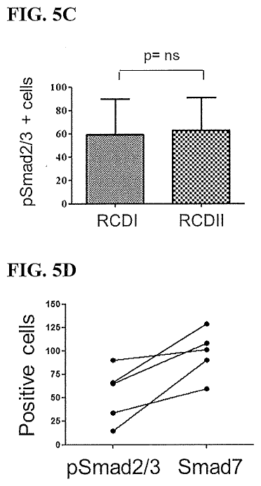

To analyze expression of active SMAD2/3 in gastrointestinal tissue, propria lamina and epithelial gastrointestinal tissue from CTR, ICD, ACD, RCDI, and RCDII patients were stained using an anti-phosphorylated SMAD2/3 (pSMAD2/3) antibody. Paraffin-embedded duodenal sections were prepared as above but were analyzed using an anti-human pSmad2/3 rabbit polyclonal antibody (Santa Cruz Biotechnology INC, Santa Cruz, Calif., USA, 1:1000 final dilution). pSMAD2/3 signal appeared less abundant in RCDI and RCDII tissue samples compared to CTR tissue samples (FIG. 5A). Isotype control staining of tissue revealed no appreciable signal. Quantification of pSMAD2/3 signal in stained tissue samples revealed that there were significantly fewer pSMAD2/3-expressing cells in tissue samples from RCD patients compared to CTR patients (FIG. 5B, CTR v. RCD, p=0.01), whereas no significant difference in the number of pSMAD2/3-expressing cells was revealed between RCDI and RCDII tissue samples (FIG. 5C). These results demonstrate that levels of active SMAD2/3 protein were significantly lower in gastrointestinal tissue of RCD patients compared to healthy control patients. Furthermore, a comparison of the number of pSMAD2/3-positive cells and SMAD7-positive cells in each RCD patient tissue sample revealed that the number of SMAD7-positive cells in each RCD patient tissue sample was higher than the number of pSMAD2/3-positive cells (FIG. 5D). This result demonstrates that SMAD7 protein expression was higher compared to active SMAD2/3 protein expression in gastrointestinal tissue of RCD patients.

### Example 4: IL-6 Induces SMAD7 Expression in Gastrointestinal Tissue

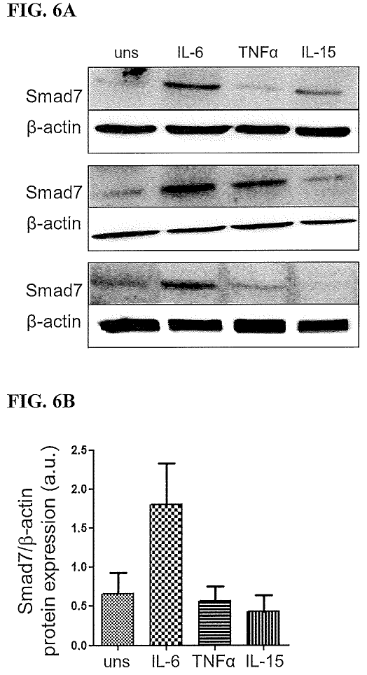

The ability of various cytokines to stimulate SMAD7 expression was analyzed. Duodenal biopsies of 5 healthy patients were placed on transwells (transwell permeable support, Costar, Corning incorporated) in 24-well plates containing X-VIVO medium supplemented with 1% penicillin/streptomycin (P/S) and 50□μg/ml gentamycin in the presence of IL-6 (10 μg/ml) (Prepotech, London, UK), tumor necrosis factor (TNF)-α (10 μg/ml) (R&D Systems, Minneapolis, Minn., USA), IL-15 (25 μg/ml) (R&D Systems, Minneapolis, Minn., USA), or medium only. The culture was performed in an organ culture chamber at 37□° C. in a 5% CO2/95% O2 atmosphere. After 24 hours, total proteins were extracted and Smad7 was detected by Western blotting as described above. IL-6 exposure consistently resulted in increased SMAD7 protein levels as revealed by Western blotting (FIG. 6A; 3 representative samples shown). Quantification of SMAD7 signal intensity (relative to β-actin control signal intensity) showed an increase in SMAD7 signal intensity following IL-6 stimulation in comparison to unstimulated control tissue (FIG. 6B). These results demonstrate that IL-6 exposure was able to stimulate increased SMAD7 protein expression in gastrointestinal tissue.

### Example 5: Smad7 Knockdown Reduces IL-6 and TNFα mRNA Expression in Gastrointestinal Tissue of Refractory Celiac Disease Patients

To analyze the effect of SMAD7 signaling on IL-6 expression in gastrointestinal tissue of refractory celiac disease patients, ex vivo mucosal explants from individual refractory celiac disease patients were cultured. Duodenal biopsies of 3 RCD patients were cultured under conditions similar to those described above, in the presence of either Smad7 sense oligonucleotide or Smad7 antisense oligonucleotide (both used at 10 μg/ml). Total RNA was extracted from each sample after 24 hours of culture, and mRNA levels of IL-6 and TNFα were analyzed, using β-actin levels as an internal control. The following PCR conditions were used: denaturation 1 minute at 95° C.; annealing 30 seconds at 61° C. for IL-6, at 62° C. for TNFα and at 60° C. for β-Actin. The following primers sequence were used: IL-6 forward, 5′-CCACTCACCTCTTCAGAACG-3 [SEQ ID NO: 9]; IL-6 reverse, 5′-GCCTCTTTGCTGCTTTCACAC-3′ [SEQ ID NO: 10]; TNFα forward, 5′-AGGCGGTGCTTGTTCCTCAG-3′ [SEQ ID NO: 11]; TNFα reverse, 5′-GGCTACAGGCTTGTCACTCG-3′ [SEQ ID NO: 12]; β-actin forward, 5′-AAGATGACCCAGATCATGTTTGAGACC-3′ [SEQ ID NO: 13]; β-actin reverse, 5′-AGCCAGTCCAGACGCAGGAT-3′ [SEQ ID NO: 14]. IL-6 mRNA and TNFα levels were decreased in mucosal explants exposed to SMAD7 antisense oligonucleotide compared to mucosal explants from the same individual patients exposed to SMAD7 sense oligonucleotide (Table 1). These results demonstrate that decreased levels of SMAD7 expression resulted in decreased IL-6 and TNFα mRNA expression in mucosal tissue of refractory celiac disease patients.

TABLE 1
Smad7 knockdown decreases IL-6 and TNFα mRNA expression

| | Smad7 sense | Smad7 antisense |
|---|---|---|
| IL-6 RNA | 371.6 | 64.6 |
| (relative expression) | 155.7 | 101.4 |
| | 29.6 | 10 |
| TNFα RNA | 223.2 | 144.6 |
| (relative expression) | 3.1 | 1.6 |
| | 5.4 | 1.2 |

### Example 6: Further Treatment of Mucosal Explants from Patients with Refractory Celiac Disease Using a SMAD7 Antisense Oligonucleotide

Mucosal explants from control patients and patient with refractory celiac disease will be established, as in Example 5. Explants will be left untreated, treated with vehicle, treated with a SMAD7 sense oligonucleotide, or treated with a SMAD7 antisense oligonucleotide, for example, Mongersen. Following treatment, levels of SMAD7, levels of inflammatory cytokines such as TNFα, and levels of TGF-β signaling components will be evaluated in explants.

### Example 7: Treatment of Mucosal Explants from Patients with Refractory Celiac Disease Using an Anti-IL-6 Therapy

To analyze the effect of IL-6 expression on TGF-β signaling activity in gastrointestinal tissue of refractory celiac disease patients, ex vivo mucosal explants from individual control patients and refractory celiac disease patients will be cultured as described in Example 5. Duodenal biopsies of patients will be left untreated, treated with vehicle, treated with a specific inhibitor of IL-6 (e.g., an IL-6 antisense oligonucleotide, a small molecule inhibitor of IL-6, or an antibody targeting IL-6) or a suitable control (e.g., a complementary IL-6 sense oligonucleotide). Following treatment, total RNA will be extracted from each sample, and mRNA levels of IL-6, various TGF-β signaling components (e.g., TGF-β1, TGF-β2, TGF-β3, SMAD2, SMAD3, SMAD4, p-SMAD2, p-SMAD3, and/or SMAD7) and/or various inhibitory cytokines (e.g., TNFα, IL-25, and/or IL-15) will be evaluated in explants.

### Example 8: Treatment of Mucosal Explants from Patients with Refractory Celiac Disease Using an Anti-TNFα Therapy

To analyze the effect of TNFα expression on TGF-β signaling activity in gastrointestinal tissue of refractory celiac disease patients, ex vivo mucosal explants from individual control patients and refractory celiac disease patients will be cultured as described in Example 5. Duodenal biopsies of patients will be left untreated, treated with vehicle, treated with a specific inhibitor of TNFα (e.g., a TNFα antisense oligonucleotide, a small molecule inhibitor of TNFα, or an antibody targeting TNFα) or a suitable control (e.g., a complementary TNFα sense oligonucleotide). Following treatment, total RNA will be extracted from each sample, and mRNA levels of TNFα, various TGF-β signaling components (e.g., TGF-β1, TGF-β2, TGF-β3, SMAD2, SMAD3, SMAD4, p-SMAD2, p-SMAD3, and/or SMAD7) and/or various inhibitory cytokines (e.g., IL-6, IL-25, and/or IL-15) will be evaluated in explants.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

Numbered clauses of the present disclosure are:

1. A method of treating or preventing celiac disease or enhancing Transforming Growth Factor-β (TGF-β) signaling in a cell of a patient with celiac disease, comprising inhibiting inhibiting Mothers Against Decapentaplegic Homolog 7 (SMAD7) in a patient suffering from celiac disease.
2. A method of treating or preventing celiac disease or enhancing TGF-β signaling in a cell of a patient with celiac disease, comprising inhibiting SMAD7 in an intestinal cell.
3. A method of treating celiac disease in a patient with celiac disease, comprising administering to the patient an effective amount of a specific inhibitor of SMAD7.
4. The method of claim 2, wherein the intestinal cell is a small intestinal cell.
5. The method of claim 2, wherein the intestinal cell is a large intestinal cell.
6. The method of claim 2, wherein the intestinal cell is a lamina propria mononuclear cell.
7. A method according to any one of the preceding claims, wherein the patient is not suffering from an inflammatory bowel disease.
8. A method according to any one of claims 1-6, wherein celiac disease is preceded by inflammatory bowel disease.
9. A method according to any one of claims 1-6, wherein the patient is suffering from an inflammatory bowel disease.
10. The method of claim 8 or 9, wherein the inflammatory bowel disease is Crohn's disease.
11. The method of claim 8 or 9, wherein the inflammatory bowel disease is ulcerative colitis.
12. The method of claim 3, wherein the inhibitor comprises a SMAD7 antisense oligonucleotide.
13. The method of claim 12, wherein the SMAD7 antisense oligonucleotide comprises an oligonucleotide selected from the group consisting of SEQ ID NO: 2, SEQ ID NO: 3, and SEQ ID NO: 4.
14. The method of claim 12, wherein SMAD7 antisense oligonucleotide comprises SEQ ID NO: 4.
15. The method claim 12, wherein the SMAD7 antisense oligonucleotide is administered parenterally.
16. The method of claim 12, wherein the SMAD7 antisense oligonucleotide is administered orally.
17. The method of claim 3, comprising administering a pharmaceutical composition comprising a SMAD7 antisense oligonucleotide and a pharmaceutically acceptable carrier.
18. The method of claim 17, wherein the SMAD7 antisense oligonucleotide is selected from the group consisting of SEQ ID NO: 2, SEQ ID NO: 3, and SEQ ID NO: 4.
19. The method of claim 18, wherein the SMAD7 antisense oligonucleotide comprises SEQ ID NO: 4.
20. The method of claim 17, wherein the pharmaceutical composition is administered parenterally.
21. The method of claim 17, wherein the pharmaceutical composition is administered orally.
22. The method of claim 17, wherein the pharmaceutical composition comprises an enteric coating comprising an ethylacrylate-methacrylic acid copolymer.
23. The method of claim 1 or 3, wherein the patient is a human.
24. The method of claim 12 or 17, comprising administering at least 100 μg of the antisense oligonucleotide.
25. The method of claim 24, comprising administering from 35 mg to 500 mg of the antisense oligonucleotide.
26. A method for treating celiac disease in a patient having celiac disease, wherein the method comprises (a) administering to the patient an initial dose of a SMAD7 antisense-oligonucleotide; (b) analyzing the level of a TGF-β (Transforming Growth Factor-β) signaling activity in the patient; and (c) if the level of TGF-β signaling activity is below normal levels of TGF-β signaling activity, then administering to the patient a subsequent dose that is greater than or equal to the initial dose, or, if the level of TGF-β signaling activity is above normal levels of TGF-β signaling activity, then administering to the patient a subsequent dose that is equal to or smaller than the initial dose.
27. A method for treating or managing celiac disease in a patient having celiac disease, wherein the method comprises (a) analyzing the level of a TGF-β signaling activity in the patient; and (b) if the level of TGF-β signaling activity is below normal levels of TGF-β signaling activity, then administering to the patient an initial dose of a SMAD7 antisense-oligonucleotide.
28. The method of claim 27, wherein the method further comprises: (c) analyzing the level of TGF-β signaling activity in the patient after said administering step; and (d) if the level of TGF-β signaling activity is below normal levels of TGF-β signaling activity then administering to the patient a subsequent dose that is greater than or equal to the initial dose, or, if the level of TGF-β signaling activity is above normal levels of TGF-β signaling activity then administering to the patient a subsequent dose that is equal to or smaller than the initial dose.
29. The method of claim 27, wherein the method further comprises: (c) analyzing the level of TGF-β signaling activity in the patient after said administering step; and (d) if the level of TGF-β signaling activity is increased after said administration step than the level of TGF-β signaling activity before said administration step, then administering to the patient a subsequent dose that is the same as the initial dose or smaller than the initial dose, or, if the level of TGF-β signaling activity is unchanged or decreased after said administration step compared to the level of TGF-β signaling activity before said administration step, then administering to the patient a subsequent dose that is the same as the initial dose or greater than the initial dose or terminating the treatment.
30. A method for treating or managing celiac disease in a patient having celiac disease, wherein the method comprises: (a) establishing a control level of a TGF-β signaling activity for the patient; (b) administering to the patient an initial dose of a SMAD7 antisense-oligonucleotide; (c) analyzing the level of TGF-β signaling activity in the patient; and (d) if the level of TGF-β signaling activity is higher than the control level, then administering to the patient a subsequent dose that is the same as the initial dose or smaller than the initial dose, or, if the level of TGF-β signaling activity is unchanged or decreased compared to the control level, then administering to the patient a subsequent dose that is the same as the initial dose or greater than the initial dose or terminating the treatment.
31. The method of claim 29 or 30, wherein, if the level of TGF-β signaling activity is at least 10%, at least 20%, at least 30%, at least 40%, at least 50%, or at least 70% increased after said administration step compared to the level of TGF-β signaling activity before said administration step, then administering to the patient a subsequent dose that is the same as the initial dose or smaller than the initial dose.
32. The method of claim 29 or 30, further comprising determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of the celiac disease for a time period of at least 1 week, at least 2 weeks, at least 3 weeks, at least 4 weeks, at least 6 weeks or at least 8 weeks, if the level of TGF-β signaling activity after said administering step is at least 10%, at least 20%, at least 30%, at least 40%, at least 50%, at least 60%, or at least 70% increased compared to the level of TGF-β signaling activity before said administration step.
33. The method of claim 26 or claim 28, wherein if the level of TGF-β signaling activity is below normal levels of TGF-β signaling activity, then administering to the patient a subsequent dose that is greater than the initial dose, or, if the level of TGF-β signaling activity is above normal levels of TGF-β signaling activity then administering to the patient a subsequent dose that is smaller than the initial dose.
34. The method of claim 28 or 30, wherein, if the subsequent dose is equal to or greater than the maximum tolerated dose (MTD), then terminating the treatment.
35. The method of claim 28 or 30, wherein the level of TGF-β signaling activity is analyzed at least 1 day, at least 3 days, at least 5 days, at least 1 week, at least 2 weeks, at least 3 weeks, at least 1 month, at least 2 months, at least 4 months, or at least 6 months after said administration step.
36. The method of claim 28 or 30, wherein the level of TGF-β signaling activity is analyzed immediately after said administration step.
37. The method of claim 28 or 30, wherein the level of TGF-β signaling activity is analyzed about 15 days or about 28 days after said administration step.
38. The methods of claim 26 or claim 27, wherein the normal levels of TGF-β signaling activity are median levels of TGF-β signaling activity in a healthy control group.
39. The methods of claim 30, wherein the control level of TGF-β signaling activity is a median level of TGF-β signaling activity in a healthy control group.
40. The method of claim 38 or 39, wherein the healthy control group and the patient having celiac disease are matched with respect to age, gender, ethnic origin, smoking habits, dietary habits, body-mass index (BMI), and/or exercise habits.
41. The method of claim 26 or claim 27, wherein the TGF-β signaling activity is analyzed by measuring the concentration of a TGF-β signaling analyte.
42. The method of claim 30, wherein the TGF-β signaling activity is analyzed by measuring the concentration of a TGF-β signaling analyte.
43. The method of claim 26, 27, or 30, wherein the initial dose is less than 100 mg/day, less than 90 mg/day, less than 80 mg/day, less than 70 mg/day, less than 60 mg/day, less than 50 mg/day, less than 40 mg/day or less than 30 mg/day.
44. The method of claim 26, 27, or 30, wherein the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day.
45. The method of claim 26, 27, or 30, wherein the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.
46. The method of claim 26, 27, or 30, wherein the initial dose is 10 mg/day, 40 mg/day, 80 mg/day, or 160 mg/day.
47. The method of claim 26 or 28, wherein, if TGF-β signaling activity levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.
48. The method of claim 30, wherein, if TGF-β signaling activity levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.
49. The method of claim 26 or 28, wherein, if TGF-β signaling activity levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.
50. The method of claim 30, wherein, if TGF-β signaling activity levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.
51. The method of claim 26, 28, or 30, wherein the initial dose is between about 10 mg/day and 100 mg/day and the subsequent dose is between about 30 mg/day and 200 mg/day.
52. The method of claim 26, 27, or 30, wherein the level of TGF-β signaling activity in the patient having celiac disease is determined in a sample obtained from the patient having celiac disease.
53. The method of claim 52, wherein the sample is a blood, serum, plasma, or intestinal tissue sample.
54. The method of claim 26, 27, or 30, wherein the level of TGF-β signaling activity is determined by immunochemistry or by nucleotide analysis.
55. The method of claim 54, wherein the level of TGF-β signaling activity is determined by an enzyme-linked immunosorbent assay (ELISA).
56. The method of claim 26, 27, or 30, further comprising determining a level of one or more additional analytes in the patient having celiac disease.
57. The method of claim 56, wherein the one or more additional analytes comprise Tumor Necrosis Factor α (TNFα), Interleukin-6 (IL-6), Interleukin-25 (IL-25), and/or Interleukin-15 (IL-15).
58. The method of claim 26, 27, or 30, wherein the SMAD7 antisense-oligonucleotide is administered orally to the patient having celiac disease.
59. The method of claim 26, 27, or 30, wherein the SMAD7 antisense oligonucleotide targets region 108-128 of human SMAD7 (SEQ ID NO: 1).
60. The method of claim 26, 27, or 30, wherein the SMAD7 antisense oligonucleotide targets nucleotides 403, 233, 294, 295, 296, 298, 299 or 533 of human SMAD7 (SEQ ID NO: 1).
61. The method of claim 26, 27, or 30, wherein the SMAD7 antisense oligonucleotide comprises the nucleotide sequence of SEQ ID NO: 2 (5′-GTCGCCCCTTCTCCCCGCAGC-3′).
62. The method of claim 26, 27, or 30, wherein the antisense oligonucleotide is a SMAD7 phosphorothioate antisense oligonucleotide comprising the following sequence: 5′-GTXGCCCCTTCTCCCXGCAG-3′ (SEQ ID NO: 3) wherein X is a nucleotide comprising 5-methyl-2′-deoxycytidine and wherein the internucleotide linkages are phosphorothioate linkages.
63. The method of claim 62, wherein the antisense oligonucleotide is a SMAD7 phosphorothioate antisense oligonucleotide comprising the following sequence: 5′-GTXGCCCCTTCTCCCXGCAGC-3′ (SEQ ID NO: 4) wherein X is a nucleotide comprising 5-methyl-2′-deoxycytidine and wherein the internucleotide linkages are phosphorothioate linkages.
64. A method for treating or managing celiac disease in a patient with celiac disease having below normal TGF-β signaling activity levels following administration of a dose of a SMAD7 antisense oligonucleotide, said method comprising administering to said patient a further dose of said oligonucleotide that is greater than or equal to the prior dose.
65. A method for treating or managing celiac disease in a patient with celiac disease having above normal TGF-β signaling activity levels following administration of a dose of SMAD7 antisense oligonucleotide, said method comprising administering to said patient a further dose of said oligonucleotide that is less than or equal to the prior dose.
66. A method of treating or managing celiac disease in a patient with celiac disease having below normal TGF-β signaling activity levels, said method comprising administering to said patient a dose of a SMAD7 antisense oligonucleotide.
67. The method of claim 66, wherein the administering is repeated until a TGF-β signaling activity level reaches a normal level.
68. A method of monitoring the treatment or management of celiac disease in a patient with celiac disease, the method comprising analyzing TGF-β signaling activity levels in the patient following each SMAD7 antisense oligonucleotide administration, wherein the absence of an increase in TGF-β signaling activity levels indicates that the treatment or management is not effective.
69. The method of claim 68, wherein TGF-β signaling activity levels are analyzed one time, two times, three times, four times, about five times, about 10 times, about 15 times, about 20 times, or about 30 times after each administration of SMAD7 antisense oligonucleotide.
70. The method of claim 68, wherein the TGF-β signaling activity levels are analyzed immediately after, about 1 hour after, about 3 hours after, about 6 hours after, about 12 hours after, about 1 day after, about 3 days after, about 1 week after, about 2 weeks after, and/or about 1 month after SMAD7 antisense oligonucleotide administration.
71. A method of treating or managing celiac disease in a patient with celiac disease having below normal levels of TGF-β signaling activity, comprising increasing the amount of a SMAD7 antisense oligonucleotide administered to the patient until TGF-β signaling activity levels in the patient increase.
72. The method of claim 71, wherein TGF-β signaling activity increases to about a normal level of TGF-β signaling activity or an above normal level of TGF-β signaling activity.
73. A SMAD7 antisense-oligonucleotide for use in a method for treating or managing celiac disease in a patient having celiac disease, wherein the method comprises analyzing the level of TGF-β signaling activity in the patient to determine appropriate levels of SMAD7 antisense oligonucleotide administration.
74. The SMAD7 antisense-oligonucleotide for use of claim 73, wherein the method comprises the steps of: (a) administering to the patient an initial dose of the SMAD7 antisense-oligonucleotide; (b) analyzing the level of a TGF-β signaling activity in the patient; and (c) if the level of TGF-β signaling activity is below normal levels of TGF-β signaling activity, then administering to the patient a subsequent dose of the SMAD7 antisense-oligonucleotide that is greater than or equal to the initial dose, or, if the level of TGF-β signaling activity is above normal levels of TGF-β signaling activity then administering to the patient a subsequent dose of the SMAD7 antisense-oligonucleotide that is equal to or smaller than the initial dose.
75. A SMAD7 antisense-oligonucleotide for use in a method for treating or managing celiac disease in a patient having celiac disease, wherein the method comprises (a) analyzing the level of TGF-β signaling activity in the patient; and (b) if the level of TGF-β signaling activity is below normal levels of TGF-β signaling activity, then administering to the patient an initial dose of the SMAD7 antisense-oligonucleotide.
76. The method of claim 41 or 42, wherein the TGF-β signaling analyte is a protein selected from the group consisting of Transforming Growth Factor-β1 (TGF-β1), Transforming Growth Factor-β2 (TGF-β2), Transforming Growth Factor-β3 (TGF-β3), Mothers Against Decapentaplegic Homolog 2 (SMAD2), Mothers Against Decapentaplegic Homolog 3 (SMAD3), Mothers Against Decapentaplegic Homolog 4 (SMAD4), phosphorylated SMAD2 (p-SMAD2), and phosphorylated SMAD3 (p-SMAD3).
77. The method of any one of claims 1-76, wherein the celiac disease is refractory celiac disease.
78. A method of treating or preventing celiac disease or enhancing TGF-β signaling in a cell of a patient with celiac disease, comprising inhibiting IL-6 in a patient suffering from celiac disease.
79. A method of treating or preventing celiac disease or enhancing TGF-β signaling in a cell of a patient with celiac disease, comprising inhibiting IL-6 in an intestinal cell.
80. A method of treating celiac disease in a patient with celiac disease, comprising administering to the patient an effective amount of a specific inhibitor of IL-6.
81. The method of claim 78, wherein the intestinal cell is a small intestinal cell.
82. The method of claim 78, wherein the intestinal cell is a large intestinal cell.
83. The method of claim 78, wherein the intestinal cell is a lamina propria mononuclear cell.
84. A method according to any one of claims 78-83, wherein the patient is not suffering from an inflammatory bowel disease.
85. A method according to any one of claims 78-83, wherein celiac disease is preceded by inflammatory bowel disease.
86. A method according to any one of claims 78-83, wherein the patient is suffering from an inflammatory bowel disease.
87. The method of claim 85 or 86, wherein the inflammatory bowel disease is Crohn's disease.
88. The method of claim 85 or 86, wherein the inflammatory bowel disease is ulcerative colitis.
89. The method of claim 80, wherein the inhibitor comprises an IL-6 antisense oligonucleotide.
90. The method claim 89, wherein the IL-6 antisense oligonucleotide is administered parenterally.
91. The method of claim 89, wherein the IL-6 antisense oligonucleotide is administered orally.
92. The method of claim 80, comprising administering a pharmaceutical composition comprising the specific inhibitor of IL-6 and a pharmaceutically acceptable carrier.
93. The method of claim 92, wherein the pharmaceutical composition is administered parenterally.
94. The method of claim 92, wherein the pharmaceutical composition is administered orally.
95. The method of claim 92, wherein the pharmaceutical composition comprises an enteric coating comprising an ethylacrylate-methacrylic acid copolymer.
96. The method of claim 78 or 80, wherein the patient is a human.
97. The method of claim 89, comprising administering at least 100 μg of the antisense oligonucleotide.
98. The method of claim 97, comprising administering from 35 mg to 500 mg of the antisense oligonucleotide.
99. A method for treating celiac disease in a patient having celiac disease, wherein the method comprises (a) administering to the patient an initial dose of specific inhibitor of IL-6; (b) analyzing the level of a TGF-β (Transforming Growth Factor-β) signaling activity in the patient; and (c) if the level of TGF-β signaling activity is below normal levels of TGF-β signaling activity, then administering to the patient a subsequent dose that is greater than or equal to the initial dose, or, if the level of TGF-β signaling activity is above normal levels of TGF-β signaling activity, then administering to the patient a subsequent dose that is equal to or smaller than the initial dose.
100. A method for treating or managing celiac disease in a patient having celiac disease, wherein the method comprises (a) analyzing the level of a TGF-β signaling activity in the patient; and (b) if the level of TGF-β signaling activity is below normal levels of TGF-β signaling activity, then administering to the patient an initial dose of a specific inhibitor of IL-6.
101. The method of claim 100, wherein the method further comprises: (c) analyzing the level of TGF-β signaling activity in the patient after said administering step; and (d) if the level of TGF-β signaling activity is below normal levels of TGF-β signaling activity then administering to the patient a subsequent dose that is greater than or equal to the initial dose, or, if the level of TGF-β signaling activity is above normal levels of TGF-β signaling activity then administering to the patient a subsequent dose that is equal to or smaller than the initial dose.
102. The method of claim 100, wherein the method further comprises: (c) analyzing the level of TGF-β signaling activity in the patient after said administering step; and (d) if the level of TGF-β signaling activity is increased after said administration step than the level of TGF-β signaling activity before said administration step, then administering to the patient a subsequent dose that is the same as the initial dose or smaller than the initial dose, or, if the level of TGF-β signaling activity is unchanged or decreased after said administration step compared to the level of TGF-β signaling activity before said administration step, then administering to the patient a subsequent dose that is the same as the initial dose or greater than the initial dose or terminating the treatment.
103. A method for treating or managing celiac disease in a patient having celiac disease, wherein the method comprises: (a) establishing a control level of a TGF-β signaling activity for the patient; (b) administering to the patient an initial dose of a specific inhibitor of IL-6; (c) analyzing the level of TGF-β signaling activity in the patient; and (d) if the level of TGF-β signaling activity is higher than the control level, then administering to the patient a subsequent dose that is the same as the initial dose or smaller than the initial dose, or, if the level of TGF-β signaling activity is unchanged or decreased compared to the control level, then administering to the patient a subsequent dose that is the same as the initial dose or greater than the initial dose or terminating the treatment.
104. The method of claim 102 or 103, wherein, if the level of TGF-β signaling activity is at least 10%, at least 20%, at least 30%, at least 40%, at least 50%, or at least 70% increased after said administration step compared to the level of TGF-β signaling activity before said administration step, then administering to the patient a subsequent dose that is the same as the initial dose or smaller than the initial dose.
105. The method of claim 102 or 103, further comprising determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of the celiac disease for a time period of at least 1 week, at least 2 weeks, at least 3 weeks, at least 4 weeks, at least 6 weeks or at least 8 weeks, if the level of TGF-β signaling activity after said administering step is at least 10%, at least 20%, at least 30%, at least 40%, at least 50%, at least 60%, or at least 70% increased compared to the level of TGF-β signaling activity before said administration step.
106. The method of claim 99 or claim 101, wherein if the level of TGF-β signaling activity is below normal levels of TGF-β signaling activity, then administering to the patient a subsequent dose that is greater than the initial dose, or, if the level of TGF-β signaling activity is above normal levels of TGF-β signaling activity then administering to the patient a subsequent dose that is smaller than the initial dose.
107. The method of claim 101 or 103, wherein, if the subsequent dose is equal to or greater than the maximum tolerated dose (MTD), then terminating the treatment.
108. The method of claim 101 or 103, wherein the level of TGF-β signaling activity is analyzed at least 1 day, at least 3 days, at least 5 days, at least 1 week, at least 2 weeks, at least 3 weeks, at least 1 month, at least 2 months, at least 4 months, or at least 6 months after said administration step.
109. The method of claim 101 or 103, wherein the level of TGF-β signaling activity is analyzed immediately after said administration step.
110. The method of claim 101 or 103, wherein the level of TGF-β signaling activity is analyzed about 15 days or about 28 days after said administration step.
111. The methods of claim 99 or claim 100, wherein the normal levels of TGF-β signaling activity are median levels of TGF-β signaling activity in a healthy control group.
112. The methods of claim 103, wherein the control level of TGF-β signaling activity is a median level of TGF-β signaling activity in a healthy control group.
113. The method of claim 111 or 112, wherein the healthy control group and the patient having celiac disease are matched with respect to age, gender, ethnic origin, smoking habits, dietary habits, body-mass index (BMI), and/or exercise habits.
114. The method of claim 99 or claim 100, wherein the TGF-β signaling activity is analyzed by measuring the concentration of a TGF-β signaling analyte.
115. The method of claim 103, wherein the TGF-β signaling activity is analyzed by measuring the concentration of a TGF-β signaling analyte.
116. The method of claim 99, 100, or 103, wherein the initial dose is less than 100 mg/day, less than 90 mg/day, less than 80 mg/day, less than 70 mg/day, less than 60 mg/day, less than 50 mg/day, less than 40 mg/day or less than 30 mg/day.
117. The method of claim 99, 100, or 103, wherein the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day.
118. The method of claim 99, 100, or 103, wherein the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.
119. The method of claim 99, 100, or 103, wherein the initial dose is 10 mg/day, 40 mg/day, 80 mg/day, or 160 mg/day.
120. The method of claim 99 or 101, wherein, if TGF-β signaling activity levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.
121. The method of claim 103, wherein, if TGF-β signaling activity levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.
122. The method of claim 99 or 101, wherein, if TGF-β signaling activity levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.
123. The method of claim 103, wherein, if TGF-β signaling activity levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.
124. The method of claim 99, 101, or 103, wherein the initial dose is between about 10 mg/day and 100 mg/day and the subsequent dose is between about 30 mg/day and 200 mg/day.
125. The method of claim 99, 100, or 103, wherein the level of TGF-β signaling activity in the patient having celiac disease is determined in a sample obtained from the patient having celiac disease.
126. The method of claim 125, wherein the sample is a blood, serum, plasma, or intestinal tissue sample.
127. The method of claim 99, 100, or 103, wherein the level of TGF-β signaling activity is determined by immunochemistry or by nucleotide analysis.
128. The method of claim 127, wherein the level of TGF-β signaling activity is determined by an enzyme-linked immunosorbent assay (ELISA).
129. The method of claim 99, 100, or 103, further comprising determining a level of one or more additional analytes in the patient having celiac disease.
130. The method of claim 129, wherein the one or more additional analytes comprise TNFα, SMAD7, IL-25, and/or IL-15.
131. The method of claim 99, 100, or 103, wherein the specific inhibitor of IL-6 is administered orally to the patient having celiac disease.
132. A method for treating or managing celiac disease in a patient with celiac disease having below normal TGF-β signaling activity levels following administration of a dose of a specific inhibitor of IL-6, said method comprising administering to said patient a further dose of said specific inhibitor of IL-6 that is greater than or equal to the prior dose.
133. A method for treating or managing celiac disease in a patient with celiac disease having above normal TGF-β signaling activity levels following administration of a dose of a specific inhibitor of IL-6, said method comprising administering to said patient a further dose of said specific inhibitor of IL-6 that is less than or equal to the prior dose.
134. A method of treating or managing celiac disease in a patient with celiac disease having below normal TGF-β signaling activity levels, said method comprising administering to said patient a dose of a specific inhibitor of IL-6.
135. The method of claim 134, wherein the administering is repeated until a TGF-β signaling activity level reaches a normal level.
136. A method of monitoring the treatment or management of celiac disease in a patient with celiac disease, the method comprising analyzing TGF-β signaling activity levels in the patient following each specific inhibitor of IL-6 administration, wherein the absence of an increase in TGF-β signaling activity levels indicates that the treatment or management is not effective.
137. The method of claim 136, wherein TGF-β signaling activity levels are analyzed one time, two times, three times, four times, about five times, about 10 times, about 15 times, about 20 times, or about 30 times after each administration of the specific inhibitor of IL-6.
138. The method of claim 136, wherein the TGF-β signaling activity levels are analyzed immediately after, about 1 hour after, about 3 hours after, about 6 hours after, about 12 hours after, about 1 day after, about 3 days after, about 1 week after, about 2 weeks after, and/or about 1 month after administration of the specific inhibitor of IL-6.
139. A method of treating or managing celiac disease in a patient with celiac disease having below normal levels of TGF-β signaling activity, comprising increasing the amount of a specific inhibitor of IL-6 administered to the patient until TGF-β signaling activity levels in the patient increase.
140. The method of claim 139, wherein TGF-β signaling activity increases to about a normal level of TGF-β signaling activity or an above normal level of TGF-β signaling activity.
141. A specific inhibitor of IL-6 for use in a method for treating or managing celiac disease in a patient having celiac disease, wherein the method comprises analyzing the level of TGF-β signaling activity in the patient to determine appropriate levels of specific inhibitor of IL-6 administration.
142. The specific inhibitor of IL-6 for use of claim 141, wherein the method comprises the steps of: (a) administering to the patient an initial dose of the specific inhibitor of IL-6; (b) analyzing the level of a TGF-β signaling activity in the patient; and (c) if the level of TGF-β signaling activity is below normal levels of TGF-β signaling activity, then administering to the patient a subsequent dose of the specific inhibitor of IL-6 that is greater than or equal to the initial dose, or, if the level of TGF-β signaling activity is above normal levels of TGF-β signaling activity then administering to the patient a subsequent dose of the specific inhibitor of IL-6 that is equal to or smaller than the initial dose.
143. A specific inhibitor of IL-6 for use in a method for treating or managing celiac disease in a patient having celiac disease, wherein the method comprises (a) analyzing the level of TGF-β signaling activity in the patient; and (b) if the level of TGF-β signaling activity is below normal levels of TGF-β signaling activity, then administering to the patient an initial dose of the specific inhibitor of IL-6.
144. The method of claim 114 or 115, wherein the TGF-β signaling analyte is a protein selected from the group consisting of TGF-β1, TGF-β2, TGF-β3, SMAD2, SMAD3, SMAD4, p-SMAD2, and p-SMAD3.
145. The method of any one of claims 78-144, wherein the celiac disease is refractory celiac disease.
146. A method of treating or preventing celiac disease or enhancing TGF-β signaling in a cell of a patient with celiac disease, comprising inhibiting TNFα in a patient suffering from celiac disease.
147. A method of treating or preventing celiac disease or enhancing TGF-β signaling in a cell of a patient with celiac disease, comprising inhibiting TNFα in an intestinal cell.
148. A method of treating celiac disease in a patient with celiac disease, comprising administering to the patient an effective amount of a specific inhibitor of TNFα.
149. The method of claim 146, wherein the intestinal cell is a small intestinal cell.
150. The method of claim 146, wherein the intestinal cell is a large intestinal cell.
151. The method of claim 146, wherein the intestinal cell is a lamina propria mononuclear cell.
152. A method according to any one of claims 146-151, wherein the patient is not suffering from an inflammatory bowel disease.
153. A method according to any one of claims 146-151, wherein celiac disease is preceded by inflammatory bowel disease.
154. A method according to any one of claims 146-151, wherein the patient is suffering from an inflammatory bowel disease.
155. The method of claim 153 or 154, wherein the inflammatory bowel disease is Crohn's disease.
156. The method of claim 153 or 154, wherein the inflammatory bowel disease is ulcerative colitis.
157. The method of claim 148, wherein the inhibitor comprises a TNFα antisense oligonucleotide.
158. The method claim 157, wherein the TNFα antisense oligonucleotide is administered parenterally.
159. The method of claim 157, wherein TNFα antisense oligonucleotide is administered orally.
160. The method of claim 148, comprising administering a pharmaceutical composition comprising a specific inhibitor of TNFα and a pharmaceutically acceptable carrier.
161. The method of claim 160, wherein the pharmaceutical composition is administered parenterally.
162. The method of claim 160, wherein the pharmaceutical composition is administered orally.
163. The method of claim 160, wherein the pharmaceutical composition comprises an enteric coating comprising an ethylacrylate-methacrylic acid copolymer.
164. The method of claim 146 or 148, wherein the patient is a human.
165. The method of claim 157, comprising administering at least 100 μg of the antisense oligonucleotide.
166. The method of claim 165, comprising administering from 35 mg to 500 mg of the antisense oligonucleotide.
167. A method for treating celiac disease in a patient having celiac disease, wherein the method comprises (a) administering to the patient an initial dose of specific inhibitor of TNFα; (b) analyzing the level of a TGF-β (Transforming Growth Factor-β) signaling activity in the patient; and (c) if the level of TGF-β signaling activity is below normal levels of TGF-β signaling activity, then administering to the patient a subsequent dose that is greater than or equal to the initial dose, or, if the level of TGF-β signaling activity is above normal levels of TGF-β signaling activity, then administering to the patient a subsequent dose that is equal to or smaller than the initial dose.
168. A method for treating or managing celiac disease in a patient having celiac disease, wherein the method comprises (a) analyzing the level of a TGF-β signaling activity in the patient; and (b) if the level of TGF-β signaling activity is below normal levels of TGF-β signaling activity, then administering to the patient an initial dose of a specific inhibitor of TNFα.
169. The method of claim 168, wherein the method further comprises: (c) analyzing the level of TGF-β signaling activity in the patient after said administering step; and (d) if the level of TGF-β signaling activity is below normal levels of TGF-β signaling activity then administering to the patient a subsequent dose that is greater than or equal to the initial dose, or, if the level of TGF-β signaling activity is above normal levels of TGF-β signaling activity then administering to the patient a subsequent dose that is equal to or smaller than the initial dose.
170. The method of claim 168, wherein the method further comprises: (c) analyzing the level of TGF-β signaling activity in the patient after said administering step; and (d) if the level of TGF-β signaling activity is increased after said administration step than the level of TGF-β signaling activity before said administration step, then administering to the patient a subsequent dose that is the same as the initial dose or smaller than the initial dose, or, if the level of TGF-β signaling activity is unchanged or decreased after said administration step compared to the level of TGF-β signaling activity before said administration step, then administering to the patient a subsequent dose that is the same as the initial dose or greater than the initial dose or terminating the treatment.
171. A method for treating or managing celiac disease in a patient having celiac disease, wherein the method comprises: (a) establishing a control level of a TGF-β signaling activity for the patient; (b) administering to the patient an initial dose of a specific inhibitor of TNFα; (c) analyzing the level of TGF-β signaling activity in the patient; and (d) if the level of TGF-β signaling activity is higher than the control level, then administering to the patient a subsequent dose that is the same as the initial dose or smaller than the initial dose, or, if the level of TGF-β signaling activity is unchanged or decreased compared to the control level, then administering to the patient a subsequent dose that is the same as the initial dose or greater than the initial dose or terminating the treatment.
172. The method of claim 170 or 171, wherein, if the level of TGF-β signaling activity is at least 10%, at least 20%, at least 30%, at least 40%, at least 50%, or at least 70% increased after said administration step compared to the level of TGF-β signaling activity before said administration step, then administering to the patient a subsequent dose that is the same as the initial dose or smaller than the initial dose.
173. The method of claim 170 or 171, further comprising determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of the celiac disease for a time period of at least 1 week, at least 2 weeks, at least 3 weeks, at least 4 weeks, at least 6 weeks or at least 8 weeks, if the level of TGF-β signaling activity after said administering step is at least 10%, at least 20%, at least 30%, at least 40%, at least 50%, at least 60%, or at least 70% increased compared to the level of TGF-β signaling activity before said administration step.
174. The method of claim 167 or claim 169, wherein if the level of TGF-β signaling activity is below normal levels of TGF-β signaling activity, then administering to the patient a subsequent dose that is greater than the initial dose, or, if the level of TGF-β signaling activity is above normal levels of TGF-β signaling activity then administering to the patient a subsequent dose that is smaller than the initial dose.
175. The method of claim 169 or 171, wherein, if the subsequent dose is equal to or greater than the maximum tolerated dose (MTD), then terminating the treatment.
176. The method of claim 169 or 171, wherein the level of TGF-β signaling activity is analyzed at least 1 day, at least 3 days, at least 5 days, at least 1 week, at least 2 weeks, at least 3 weeks, at least 1 month, at least 2 months, at least 4 months, or at least 6 months after said administration step.
177. The method of claim 169 or 171, wherein the level of TGF-β signaling activity is analyzed immediately after said administration step.
178. The method of claim 169 or 171, wherein the level of TGF-β signaling activity is analyzed about 15 days or about 28 days after said administration step.
179. The methods of claim 173 or claim 174, wherein the normal levels of TGF-β signaling activity are median levels of TGF-β signaling activity in a healthy control group.
180. The methods of claim 171, wherein the control level of TGF-β signaling activity is a median level of TGF-β signaling activity in a healthy control group.
181. The method of claim 179 or 180, wherein the healthy control group and the patient having celiac disease are matched with respect to age, gender, ethnic origin, smoking habits, dietary habits, body-mass index (BMI), and/or exercise habits.
182. The method of claim 167 or claim 168, wherein the TGF-β signaling activity is analyzed by measuring the concentration of a TGF-β signaling analyte.
183. The method of claim 171, wherein the TGF-β signaling activity is analyzed by measuring the concentration of a TGF-β signaling analyte.
184. The method of claim 167, 168, or 171, wherein the initial dose is less than 100 mg/day, less than 90 mg/day, less than 80 mg/day, less than 70 mg/day, less than 60 mg/day, less than 50 mg/day, less than 40 mg/day or less than 30 mg/day.
185. The method of claim 167, 168, or 171, wherein the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day.
186. The method of claim 167, 168, or 171, wherein the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.
187. The method of claim 167, 168, or 171, wherein the initial dose is 10 mg/day, 40 mg/day, 80 mg/day, or 160 mg/day.
188. The method of claim 167 or 169, wherein, if TGF-β signaling activity levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.
189. The method of claim 171, wherein, if TGF-β signaling activity levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.
190. The method of claim 167 or 169, wherein, if TGF-β signaling activity levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.
191. The method of claim 171, wherein, if TGF-β signaling activity levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.
192. The method of claim 167, 169, or 171, wherein the initial dose is between about 10 mg/day and 100 mg/day and the subsequent dose is between about 30 mg/day and 200 mg/day.
193. The method of claim 167, 168, or 171, wherein the level of TGF-β signaling activity in the patient having celiac disease is determined in a sample obtained from the patient having celiac disease.
194. The method of claim 193, wherein the sample is a blood, serum, plasma, or intestinal tissue sample.
195. The method of claim 167, 168, or 171, wherein the level of TGF-β signaling activity is determined by immunochemistry or by nucleotide analysis.
196. The method of claim 195, wherein the level of TGF-β signaling activity is determined by an enzyme-linked immunosorbent assay (ELISA).
197. The method of claim 167, 168, or 171, further comprising determining a level of one or more additional analytes in the patient having celiac disease.
198. The method of claim 197, wherein the one or more additional analytes comprise IL-6, SMAD7, IL-25, and/or IL-15.
199. The method of claim 167, 168, or 171, wherein the specific inhibitor of TNFα is administered orally to the patient having celiac disease.
200. A method for treating or managing celiac disease in a patient with celiac disease having below normal TGF-β signaling activity levels following administration of a dose of a specific inhibitor of TNFα, said method comprising administering to said patient a further dose of said specific inhibitor of TNFα that is greater than or equal to the prior dose.
201. A method for treating or managing celiac disease in a patient with celiac disease having above normal TGF-β signaling activity levels following administration of a dose of a specific inhibitor of TNFα, said method comprising administering to said patient a further dose of said specific inhibitor of TNFα that is less than or equal to the prior dose.
202. A method of treating or managing celiac disease in a patient with celiac disease having below normal TGF-β signaling activity levels, said method comprising administering to said patient a dose of a specific inhibitor of TNFα.
203. The method of claim 202, wherein the administering is repeated until a TGF-β signaling activity level reaches a normal level.
204. A method of monitoring the treatment or management of celiac disease in a patient with celiac disease, the method comprising analyzing TGF-β signaling activity levels in the patient following each specific inhibitor of TNFα administration, wherein the absence of an increase in TGF-β signaling activity levels indicates that the treatment or management is not effective.
205. The method of claim 204, wherein TGF-β signaling activity levels are analyzed one time, two times, three times, four times, about five times, about 10 times, about 15 times, about 20 times, or about 30 times after each administration of the specific inhibitor of TNFα.
206. The method of claim 204, wherein the TGF-β signaling activity levels are analyzed immediately after, about 1 hour after, about 3 hours after, about 6 hours after, about 12 hours after, about 1 day after, about 3 days after, about 1 week after, about 2 weeks after, and/or about 1 month after administration of the specific inhibitor of TNFα.
207. A method of treating or managing celiac disease in a patient with celiac disease having below normal levels of TGF-β signaling activity, comprising increasing the amount of a specific inhibitor of TNFα administered to the patient until TGF-β signaling activity levels in the patient increase.
208. The method of claim 207, wherein TGF-β signaling activity increases to about a normal level of TGF-β signaling activity or an above normal level of TGF-β signaling activity.
209. A specific inhibitor of TNFα for use in a method for treating or managing celiac disease in a patient having celiac disease, wherein the method comprises analyzing the level of TGF-β signaling activity in the patient to determine appropriate levels of specific inhibitor of TNFα administration.
210. The specific inhibitor of TNFα for use of claim 209, wherein the method comprises the steps of: (a) administering to the patient an initial dose of the specific inhibitor of TNFα; (b) analyzing the level of a TGF-β signaling activity in the patient; and (c) if the level of TGF-β signaling activity is below normal levels of TGF-β signaling activity, then administering to the patient a subsequent dose of the specific inhibitor of TNFα that is greater than or equal to the initial dose, or, if the level of TGF-β signaling activity is above normal levels of TGF-β signaling activity then administering to the patient a subsequent dose of the specific inhibitor of TNFα that is equal to or smaller than the initial dose.
211. A specific inhibitor of TNFα for use in a method for treating or managing celiac disease in a patient having celiac disease, wherein the method comprises (a) analyzing the level of TGF-β signaling activity in the patient; and (b) if the level of TGF-β signaling activity is below normal levels of TGF-β signaling activity, then administering to the patient an initial dose of the specific inhibitor of TNFα.
212. The method of claim 182 or 183, wherein the TGF-β signaling analyte is a protein selected from the group consisting of TGF-β1, TGF-β2, TGF-β3, SMAD2, SMAD3, SMAD4, p-SMAD2, and p-SMAD3.
213. The method of any one of claims 146-212, wherein the celiac disease is refractory celiac disease.
214. A method for treating celiac disease in a patient having celiac disease, wherein the method comprises (a) administering to the patient an initial dose of a SMAD7 antisense-oligonucleotide; (b) analyzing the level of SMAD7, IL-6, and/or TNFα in the patient; and (c) if the level of SMAD7, IL-6, and/or TNFα is above normal levels of SMAD7, IL-6, and/or TNFα, then administering to the patient a subsequent dose that is greater than or equal to the initial dose, or, if the level SMAD7, IL-6, and/or TNFα is below normal levels of SMAD7, IL-6, and/or TNFα, then administering to the patient a subsequent dose that is equal to or smaller than the initial dose.
215. A method for treating or managing celiac disease in a patient having celiac disease, wherein the method comprises (a) analyzing the level of a SMAD7, IL-6, and/or TNFα in the patient; and (b) if the level of SMAD7, IL-6, and/or TNFα is above normal levels of SMAD7, IL-6, and/or TNFα, then administering to the patient an initial dose of a SMAD7 antisense-oligonucleotide.
216. The method of claim 215, wherein the method further comprises: (c) analyzing the level of SMAD7, IL-6, and/or TNFα in the patient after said administering step; and (d) if the level of SMAD7, IL-6, and/or TNFα is above normal levels of SMAD7, IL-6, and/or TNFα then administering to the patient a subsequent dose that is greater than or equal to the initial dose, or, if the level of SMAD7, IL-6, and/or TNFα is below normal levels of SMAD7, IL-6, and/or TNFα then administering to the patient a subsequent dose that is equal to or smaller than the initial dose.
217. The method of claim 215, wherein the method further comprises: (c) analyzing the level of SMAD7, IL-6, and/or TNFα in the patient after said administering step; and (d) if the level of SMAD7, IL-6, and/or TNFα is decreased after said administration step compared to the level of SMAD7, IL-6, and/or TNFα before said administration step, then administering to the patient a subsequent dose that is the same as the initial dose or smaller than the initial dose, or, if the level of SMAD7, IL-6, and/or TNFα is unchanged or increased after said administration step compared to the level of SMAD7, IL-6, and/or TNFα before said administration step, then administering to the patient a subsequent dose that is the same as the initial dose or greater than the initial dose or terminating the treatment.
218. A method for treating or managing celiac disease in a patient having celiac disease, wherein the method comprises: (a) establishing a control level of a SMAD7, IL-6, and/or TNFα for the patient; (b) administering to the patient an initial dose of a SMAD7 antisense-oligonucleotide; (c) analyzing the level of SMAD7, IL-6, and/or TNFα in the patient; and (d) if the level of SMAD7, IL-6, and/or TNFα is lower than the control level, then administering to the patient a subsequent dose that is the same as the initial dose or smaller than the initial dose, or, if the level of SMAD7, IL-6, and/or TNFα is unchanged or increased compared to the control level, then administering to the patient a subsequent dose that is the same as the initial dose or greater than the initial dose or terminating the treatment.
219. The method of claim 217 or 218, wherein, if the level of SMAD7, IL-6, and/or TNFα is at least 10%, at least 20%, at least 30%, at least 40%, at least 50%, or at least 70% decreased after said administration step compared to the level of SMAD7, IL-6, and/or TNFα before said administration step, then administering to the patient a subsequent dose that is the same as the initial dose or smaller than the initial dose.
220. The method of claim 217 or 218, further comprising determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of the celiac disease for a time period of at least 1 week, at least 2 weeks, at least 3 weeks, at least 4 weeks, at least 6 weeks or at least 8 weeks, if the level of SMAD7, IL-6, and/or TNFα after said administering step is decreased at least 10%, at least 20%, at least 30%, at least 40%, at least 50%, at least 60%, or at least 70% compared to the level of SMAD7, IL-6, and/or TNFα before said administration step.
221. The method of claim 214 or claim 216, wherein if the level of SMAD7, IL-6, and/or TNFα is above normal levels of SMAD7, IL-6, and/or TNFα, then administering to the patient a subsequent dose that is greater than the initial dose, or, if the level of SMAD7, IL-6, and/or TNFα is below normal levels of SMAD7, IL-6, and/or TNFα then administering to the patient a subsequent dose that is smaller than the initial dose.
222. The method of claim 216 or 218, wherein, if the subsequent dose is equal to or greater than the maximum tolerated dose (MTD), then terminating the treatment.
223. The method of claim 216 or 218, wherein the level of SMAD7, IL-6, and/or TNFα is analyzed at least 1 day, at least 3 days, at least 5 days, at least 1 week, at least 2 weeks, at least 3 weeks, at least 1 month, at least 2 months, at least 4 months, or at least 6 months after said administration step.
224. The method of claim 216 or 218, wherein the level of SMAD7, IL-6, and/or TNFα is analyzed immediately after said administration step.
225. The method of claim 216 or 218, wherein the level of SMAD7, IL-6, and/or TNFα is analyzed about 15 days or about 28 days after said administration step.
226. The methods of claim 214 or claim 215, wherein the normal levels of SMAD7, IL-6, and/or TNFα are median levels of SMAD7, IL-6, and/or TNFα in a healthy control group.
227. The methods of claim 218, wherein the control level of SMAD7, IL-6, and/or TNFα is a median level of SMAD7, IL-6, and/or TNFα in a healthy control group.
228. The method of claim 226 or 227, wherein the healthy control group and the patient having celiac disease are matched with respect to age, gender, ethnic origin, smoking habits, dietary habits, body-mass index (BMI), and/or exercise habits.
229. The method of claim 214 or claim 215, wherein the SMAD7, IL-6, and/or TNFα is analyzed by measuring the concentration of SMAD7 protein, SMAD7 mRNA, IL-6 protein, IL-6 mRNA, TNFα protein, and/or TNFα mRNA.
230. The method of claim 218, wherein the SMAD7, IL-6, and/or TNFα is analyzed by measuring the concentration of SMAD7 protein, SMAD7 mRNA, IL-6 protein, IL-6 mRNA, TNFα protein, and/or TNFα mRNA.
231. The method of claim 214, 215, or 218, wherein the initial dose is less than 100 mg/day, less than 90 mg/day, less than 80 mg/day, less than 70 mg/day, less than 60 mg/day, less than 50 mg/day, less than 40 mg/day or less than 30 mg/day.
232. The method of claim 214, 215, or 218, wherein the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day.
233. The method of claim 214, 215, or 218, wherein the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.
234. The method of claim 214, 215, or 218, wherein the initial dose is 10 mg/day, 40 mg/day, 80 mg/day, or 160 mg/day.
235. The method of claim 214 or 216, wherein, if SMAD7, IL-6, and/or TNFα levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.
236. The method of claim 218, wherein, if SMAD7, IL-6, and/or TNFα levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.
237. The method of claim 214 or 16 wherein, if SMAD7, IL-6, and/or TNFα levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.
238. The method of claim 218, wherein, if SMAD7, IL-6, and/or TNFα levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.
239. The method of claim 214, 216, or 218, wherein the initial dose is between about 10 mg/day and 100 mg/day and the subsequent dose is between about 30 mg/day and 200 mg/day.
240. The method of claim 214, 215, or 218, wherein the level of SMAD7, IL-6, and/or TNFα in the patient having celiac disease is determined in a sample obtained from the patient having celiac disease.
241. The method of claim 240, wherein the sample is a blood, serum, plasma, or intestinal tissue sample.
242. The method of claim 214, 215, or 218, wherein the level of SMAD7, IL-6, and/or TNFα is determined by immunochemistry or by nucleotide analysis.
243. The method of claim 242, wherein the level of SMAD7, IL-6, and/or TNFα is determined by an enzyme-linked immunosorbent assay (ELISA).
244. The method of claim 214, 215, or 218, further comprising determining a level of one or more additional analytes in the patient having celiac disease.
245. The method of claim 244, wherein the one or more additional analytes comprise IL-25 and/or IL-15.
246. The method of claim 214, 215, or 218, wherein the SMAD7 antisense-oligonucleotide is administered orally to the patient having celiac disease.
247. The method of claim 214, 215, or 218, wherein the SMAD7 antisense oligonucleotide targets region 108-128 of human SMAD7 (SEQ ID NO: 1).
248. The method of claim 214, 215, or 218, wherein the SMAD7 antisense oligonucleotide targets nucleotides 403, 233, 294, 295, 296, 298, 299 or 533 of human SMAD7 (SEQ ID NO: 1).
249. The method of claim 214, 215, or 218, wherein the SMAD7 antisense oligonucleotide comprises the nucleotide sequence of SEQ ID NO: 2 (5′-GTCGCCCCTTCTCCCCGCAGC-3′).
250. The method of claim 214, 215, or 218, wherein the antisense oligonucleotide is a SMAD7 phosphorothioate antisense oligonucleotide comprising the following sequence: 5′-GTXGCCCCTTCTCCCXGCAG-3′ (SEQ ID NO: 3) wherein X is a nucleotide comprising 5-methyl-2′-deoxycytidine and wherein the internucleotide linkages are phosphorothioate linkages.
251. The method of claim 250, wherein the antisense oligonucleotide is a SMAD7 phosphorothioate antisense oligonucleotide comprising the following sequence: 5′-GTXGCCCCTTCTCCCXGCAGC-3′ (SEQ ID NO: 4) wherein X is a nucleotide comprising 5-methyl-2′-deoxycytidine and wherein the internucleotide linkages are phosphorothioate linkages.
252. A method for treating or managing celiac disease in a patient with celiac disease having above normal SMAD7, IL-6, and/or TNFα levels following administration of a dose of a SMAD7 antisense oligonucleotide, said method comprising administering to said patient a further dose of said oligonucleotide that is greater than or equal to the prior dose.
253. A method for treating or managing celiac disease in a patient with celiac disease having below normal SMAD7, IL-6, and/or TNFα levels following administration of a dose of SMAD7 antisense oligonucleotide, said method comprising administering to said patient a further dose of said oligonucleotide that is less than or equal to the prior dose.
254. A method of treating or managing celiac disease in a patient with celiac disease having above normal SMAD7, IL-6, and/or TNFα levels, said method comprising administering to said patient a dose of a SMAD7 antisense oligonucleotide.
255. The method of claim 254, wherein the administering is repeated until a SMAD7, IL-6, and/or TNFα level reaches a normal level.
256. A method of monitoring the treatment or management of celiac disease in a patient with celiac disease, the method comprising analyzing SMAD7, IL-6, and/or TNFα levels in the patient following each SMAD7 antisense oligonucleotide administration, wherein the absence of an decrease in SMAD7, IL-6, and/or TNFα levels indicates that the treatment or management is not effective.
257. The method of claim 256, wherein SMAD7, IL-6, and/or TNFα levels are analyzed one time, two times, three times, four times, about five times, about 10 times, about 15 times, about 20 times, or about 30 times after each administration of SMAD7 antisense oligonucleotide.
258. The method of claim 256, wherein the SMAD7, IL-6, and/or TNFα levels are analyzed immediately after, about 1 hour after, about 3 hours after, about 6 hours after, about 12 hours after, about 1 day after, about 3 days after, about 1 week after, about 2 weeks after, and/or about 1 month after SMAD7 antisense oligonucleotide administration.
259. A method of treating or managing celiac disease in a patient with celiac disease having above normal levels of SMAD7, IL-6, and/or TNFα, comprising increasing the amount of a SMAD7 antisense oligonucleotide administered to the patient until SMAD7, IL-6, and/or TNFα levels in the patient decrease.
260. The method of claim 259, wherein SMAD7, IL-6, and/or TNFα decreases to about a normal level of SMAD7, IL-6, and/or TNFα or a below normal level of SMAD7, IL-6, and/or TNFα.
261. A SMAD7 antisense-oligonucleotide for use in a method for treating or managing celiac disease in a patient having celiac disease, wherein the method comprises analyzing the level of SMAD7, IL-6, and/or TNFα in the patient to determine appropriate levels of SMAD7 antisense oligonucleotide administration.
262. The SMAD7 antisense-oligonucleotide for use of claim 261, wherein the method comprises the steps of: (a) administering to the patient an initial dose of the SMAD7 antisense-oligonucleotide; (b) analyzing the level of a SMAD7, IL-6, and/or TNFα in the patient; and (c) if the level of SMAD7, IL-6, and/or TNFα is above normal levels of SMAD7, IL-6, and/or TNFα, then administering to the patient a subsequent dose of the SMAD7 antisense-oligonucleotide that is greater than or equal to the initial dose, or, if the level of SMAD7, IL-6, and/or TNFα is below normal levels of SMAD7, IL-6, and/or TNFα then administering to the patient a subsequent dose of the SMAD7 antisense-oligonucleotide that is equal to or smaller than the initial dose.
263. A SMAD7 antisense-oligonucleotide for use in a method for treating or managing celiac disease in a patient having celiac disease, wherein the method comprises (a) analyzing the level of SMAD7, IL-6, and/or TNFα in the patient; and (b) if the level of SMAD7, IL-6, and/or TNFα is above normal levels of SMAD7, IL-6, and/or TNFα, then administering to the patient an initial dose of the SMAD7 antisense-oligonucleotide.
264. The method of any one of claims 214-263, wherein the celiac disease is refractory celiac disease.
265. A method for treating celiac disease in a patient having celiac disease, wherein the method comprises (a) administering to the patient an initial dose of a specific inhibitor of IL-6; (b) analyzing the level of SMAD7, IL-6, and/or TNFα in the patient; and (c) if the level of SMAD7, IL-6, and/or TNFα is above normal levels of SMAD7, IL-6, and/or TNFα, then administering to the patient a subsequent dose that is greater than or equal to the initial dose, or, if the level SMAD7, IL-6, and/or TNFα is below normal levels of SMAD7, IL-6, and/or TNFα, then administering to the patient a subsequent dose that is equal to or smaller than the initial dose.
266. A method for treating or managing celiac disease in a patient having celiac disease, wherein the method comprises (a) analyzing the level of a SMAD7, IL-6, and/or TNFα in the patient; and (b) if the level of SMAD7, IL-6, and/or TNFα is above normal levels of SMAD7, IL-6, and/or TNFα, then administering to the patient an initial dose of a specific inhibitor of IL-6.
267. The method of claim 266, wherein the method further comprises: (c) analyzing the level of SMAD7, IL-6, and/or TNFα in the patient after said administering step; and (d) if the level of SMAD7, IL-6, and/or TNFα is above normal levels of SMAD7, IL-6, and/or TNFα then administering to the patient a subsequent dose that is greater than or equal to the initial dose, or, if the level of SMAD7, IL-6, and/or TNFα is below normal levels of SMAD7, IL-6, and/or TNFα then administering to the patient a subsequent dose that is equal to or smaller than the initial dose.
268. The method of claim 266, wherein the method further comprises: (c) analyzing the level of SMAD7, IL-6, and/or TNFα in the patient after said administering step; and (d) if the level of SMAD7, IL-6, and/or TNFα is decreased after said administration step compared to the level of SMAD7, IL-6, and/or TNFα before said administration step, then administering to the patient a subsequent dose that is the same as the initial dose or smaller than the initial dose, or, if the level of SMAD7, IL-6, and/or TNFα is unchanged or increased after said administration step compared to the level of SMAD7, IL-6, and/or TNFα before said administration step, then administering to the patient a subsequent dose that is the same as the initial dose or greater than the initial dose or terminating the treatment.
269. A method for treating or managing celiac disease in a patient having celiac disease, wherein the method comprises: (a) establishing a control level of a SMAD7, IL-6, and/or TNFα for the patient; (b) administering to the patient an initial dose of a specific inhibitor of IL-6; (c) analyzing the level of SMAD7, IL-6, and/or TNFα in the patient; and (d) if the level of SMAD7, IL-6, and/or TNFα is lower than the control level, then administering to the patient a subsequent dose that is the same as the initial dose or smaller than the initial dose, or, if the level of SMAD7, IL-6, and/or TNFα is unchanged or increased compared to the control level, then administering to the patient a subsequent dose that is the same as the initial dose or greater than the initial dose or terminating the treatment.
270. The method of claim 268 or 269, wherein, if the level of SMAD7, IL-6, and/or TNFα is at least 10%, at least 20%, at least 30%, at least 40%, at least 50%, or at least 70% decreased after said administration step compared to the level of SMAD7, IL-6, and/or TNFα before said administration step, then administering to the patient a subsequent dose that is the same as the initial dose or smaller than the initial dose.
271. The method of claim 268 or 269, further comprising determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of the celiac disease for a time period of at least 1 week, at least 2 weeks, at least 3 weeks, at least 4 weeks, at least 6 weeks or at least 8 weeks, if the level of SMAD7, IL-6, and/or TNFα after said administering step is decreased at least 10%, at least 20%, at least 30%, at least 40%, at least 50%, at least 60%, or at least 70% compared to the level of SMAD7, IL-6, and/or TNFα before said administration step.
272. The method of claim 265 or claim 267, wherein if the level of SMAD7, IL-6, and/or TNFα is above normal levels of SMAD7, IL-6, and/or TNFα, then administering to the patient a subsequent dose that is greater than the initial dose, or, if the level of SMAD7, IL-6, and/or TNFα is below normal levels of SMAD7, IL-6, and/or TNFα then administering to the patient a subsequent dose that is smaller than the initial dose.
273. The method of claim 267 or 269, wherein, if the subsequent dose is equal to or greater than the maximum tolerated dose (MTD), then terminating the treatment.
274. The method of claim 267 or 269, wherein the level of SMAD7, IL-6, and/or TNFα is analyzed at least 1 day, at least 3 days, at least 5 days, at least 1 week, at least 2 weeks, at least 3 weeks, at least 1 month, at least 2 months, at least 4 months, or at least 6 months after said administration step.
275. The method of claim 267 or 269, wherein the level of SMAD7, IL-6, and/or TNFα is analyzed immediately after said administration step.
276. The method of claim 267 or 269, wherein the level of SMAD7, IL-6, and/or TNFα is analyzed about 15 days or about 28 days after said administration step.
277. The methods of claim 265 or claim 266, wherein the normal levels of SMAD7, IL-6, and/or TNFα are median levels of SMAD7, IL-6, and/or TNFα in a healthy control group.
278. The methods of claim 269, wherein the control level of SMAD7, IL-6, and/or TNFα is a median level of SMAD7, IL-6, and/or TNFα in a healthy control group.
279. The method of claim 277 or 278, wherein the healthy control group and the patient having celiac disease are matched with respect to age, gender, ethnic origin, smoking habits, dietary habits, body-mass index (BMI), and/or exercise habits.
280. The method of claim 265 or claim 266, wherein the SMAD7, IL-6, and/or TNFα is analyzed by measuring the concentration of SMAD7 protein, SMAD7 mRNA, IL-6 protein, IL-6 mRNA, TNFα protein, and/or TNFα mRNA.
281. The method of claim 269, wherein the SMAD7, IL-6, and/or TNFα is analyzed by measuring the concentration of SMAD7 protein, SMAD7 mRNA, IL-6 protein, IL-6 mRNA, TNFα protein, and/or TNFα mRNA.
282. The method of claim 265, 266, or 269, wherein the initial dose is less than 100 mg/day, less than 90 mg/day, less than 80 mg/day, less than 70 mg/day, less than 60 mg/day, less than 50 mg/day, less than 40 mg/day or less than 30 mg/day.
283. The method of claim 265, 266, or 269, wherein the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day.
284. The method of claim 265, 266, or 269, wherein the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.
285. The method of claim 265, 266, or 269, wherein the initial dose is 10 mg/day, 40 mg/day, 80 mg/day, or 160 mg/day.
286. The method of claim 265 or 267, wherein, if SMAD7, IL-6, and/or TNFα levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.
287. The method of claim 269, wherein, if SMAD7, IL-6, and/or TNFα levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.
288. The method of claim 265 or 267 wherein, if SMAD7, IL-6, and/or TNFα levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.
289. The method of claim 269, wherein, if SMAD7, IL-6, and/or TNFα levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.
290. The method of claim 265, 267, or 269, wherein the initial dose is between about 10 mg/day and 100 mg/day and the subsequent dose is between about 30 mg/day and 200 mg/day.
291. The method of claim 265, 266, or 269, wherein the level of SMAD7, IL-6, and/or TNFα in the patient having celiac disease is determined in a sample obtained from the patient having celiac disease.
292. The method of claim 291, wherein the sample is a blood, serum, plasma, or intestinal tissue sample.
293. The method of claim 265, 266, or 269, wherein the level of SMAD7, IL-6, and/or TNFα is determined by immunochemistry or by nucleotide analysis.
294. The method of claim 293, wherein the level of SMAD7, IL-6, and/or TNFα is determined by an enzyme-linked immunosorbent assay (ELISA).
295. The method of claim 265, 266, or 269, further comprising determining a level of one or more additional analytes in the patient having celiac disease.
296. The method of claim 295, wherein the one or more additional analytes comprise IL-25 and/or IL-15.
297. The method of claim 265, 266, or 269, wherein the specific inhibitor of IL-6 is administered orally to the patient having celiac disease.
298. A method for treating or managing celiac disease in a patient with celiac disease having above normal SMAD7, IL-6, and/or TNFα levels following administration of a dose of a specific inhibitor of IL-6, said method comprising administering to said patient a further dose of said specific inhibitor of IL-6 that is greater than or equal to the prior dose.
299. A method for treating or managing celiac disease in a patient with celiac disease having below normal SMAD7, IL-6, and/or TNFα levels following administration of a dose of specific inhibitor of IL-6, said method comprising administering to said patient a further dose of said specific inhibitor of IL-6 that is less than or equal to the prior dose.
300. A method of treating or managing celiac disease in a patient with celiac disease having above normal SMAD7, IL-6, and/or TNFα levels, said method comprising administering to said patient a dose of a specific inhibitor of IL-6.
301. The method of claim 300, wherein the administering is repeated until a SMAD7, IL-6, and/or TNFα level reaches a normal level.
302. A method of monitoring the treatment or management of celiac disease in a patient with celiac disease, the method comprising analyzing SMAD7, IL-6, and/or TNFα levels in the patient following each specific inhibitor of IL-6 administration, wherein the absence of an decrease in SMAD7, IL-6, and/or TNFα levels indicates that the treatment or management is not effective.
303. The method of claim 302, wherein SMAD7, IL-6, and/or TNFα levels are analyzed one time, two times, three times, four times, about five times, about 10 times, about 15 times, about 20 times, or about 30 times after each administration of specific inhibitor of IL-6.
304. The method of claim 302, wherein the SMAD7, IL-6, and/or TNFα levels are analyzed immediately after, about 1 hour after, about 3 hours after, about 6 hours after, about 12 hours after, about 1 day after, about 3 days after, about 1 week after, about 2 weeks after, and/or about 1 month after specific inhibitor of IL-6 administration.
305. A method of treating or managing celiac disease in a patient with celiac disease having above normal levels of SMAD7, IL-6, and/or TNFα, comprising increasing the amount of a specific inhibitor of IL-6 administered to the patient until SMAD7, IL-6, and/or TNFα levels in the patient decrease.
306. The method of claim 305, wherein SMAD7, IL-6, and/or TNFα decreases to about a normal level of SMAD7, IL-6, and/or TNFα or a below normal level of SMAD7, IL-6, and/or TNFα.
307. A specific inhibitor of IL-6 for use in a method for treating or managing celiac disease in a patient having celiac disease, wherein the method comprises analyzing the level of SMAD7, IL-6, and/or TNFα in the patient to determine appropriate levels of specific inhibitor of IL-6 administration.
308. The specific inhibitor of IL-6 for use of claim 307, wherein the method comprises the steps of: (a) administering to the patient an initial dose of the specific inhibitor of IL-6; (b) analyzing the level of a SMAD7, IL-6, and/or TNFα in the patient; and (c) if the level of SMAD7, IL-6, and/or TNFα is above normal levels of SMAD7, IL-6, and/or TNFα, then administering to the patient a subsequent dose of the specific inhibitor of IL-6 that is greater than or equal to the initial dose, or, if the level of SMAD7, IL-6, and/or TNFα is below normal levels of SMAD7, IL-6, and/or TNFα then administering to the patient a subsequent dose of the specific inhibitor of IL-6 that is equal to or smaller than the initial dose.
309. A specific inhibitor of IL-6 for use in a method for treating or managing celiac disease in a patient having celiac disease, wherein the method comprises (a) analyzing the level of SMAD7, IL-6, and/or TNFα in the patient; and (b) if the level of SMAD7, IL-6, and/or TNFα is above normal levels of SMAD7, IL-6, and/or TNFα, then administering to the patient an initial dose of the specific inhibitor of IL-6.
310. The method of any one of claims 265-309, wherein the celiac disease is refractory celiac disease.
311. A method for treating celiac disease in a patient having celiac disease, wherein the method comprises (a) administering to the patient an initial dose of a specific inhibitor of TNFα; (b) analyzing the level of SMAD7, IL-6, and/or TNFα in the patient; and (c) if the level of SMAD7, IL-6, and/or TNFα is above normal levels of SMAD7, IL-6, and/or TNFα, then administering to the patient a subsequent dose that is greater than or equal to the initial dose, or, if the level SMAD7, IL-6, and/or TNFα is below normal levels of SMAD7, IL-6, and/or TNFα, then administering to the patient a subsequent dose that is equal to or smaller than the initial dose.
312. A method for treating or managing celiac disease in a patient having celiac disease, wherein the method comprises (a) analyzing the level of a SMAD7, IL-6, and/or TNFα in the patient; and (b) if the level of SMAD7, IL-6, and/or TNFα is above normal levels of SMAD7, IL-6, and/or TNFα, then administering to the patient an initial dose of a specific inhibitor of TNFα.
313. The method of claim 312, wherein the method further comprises: (c) analyzing the level of SMAD7, IL-6, and/or TNFα in the patient after said administering step; and (d) if the level of SMAD7, IL-6, and/or TNFα is above normal levels of SMAD7, IL-6, and/or TNFα then administering to the patient a subsequent dose that is greater than or equal to the initial dose, or, if the level of SMAD7, IL-6, and/or TNFα is below normal levels of SMAD7, IL-6, and/or TNFα then administering to the patient a subsequent dose that is equal to or smaller than the initial dose.
314. The method of claim 312, wherein the method further comprises: (c) analyzing the level of SMAD7, IL-6, and/or TNFα in the patient after said administering step; and (d) if the level of SMAD7, IL-6, and/or TNFα is decreased after said administration step compared to the level of SMAD7, IL-6, and/or TNFα before said administration step, then administering to the patient a subsequent dose that is the same as the initial dose or smaller than the initial dose, or, if the level of SMAD7, IL-6, and/or TNFα is unchanged or increased after said administration step compared to the level of SMAD7, IL-6, and/or TNFα before said administration step, then administering to the patient a subsequent dose that is the same as the initial dose or greater than the initial dose or terminating the treatment.
315. A method for treating or managing celiac disease in a patient having celiac disease, wherein the method comprises: (a) establishing a control level of a SMAD7, IL-6, and/or TNFα for the patient; (b) administering to the patient an initial dose of a specific inhibitor of TNFα; (c) analyzing the level of SMAD7, IL-6, and/or TNFα in the patient; and (d) if the level of SMAD7, IL-6, and/or TNFα is lower than the control level, then administering to the patient a subsequent dose that is the same as the initial dose or smaller than the initial dose, or, if the level of SMAD7, IL-6, and/or TNFα is unchanged or increased compared to the control level, then administering to the patient a subsequent dose that is the same as the initial dose or greater than the initial dose or terminating the treatment.
316. The method of claim 314 or 315, wherein, if the level of SMAD7, IL-6, and/or TNFα is at least 10%, at least 20%, at least 30%, at least 40%, at least 50%, or at least 70% decreased after said administration step compared to the level of SMAD7, IL-6, and/or TNFα before said administration step, then administering to the patient a subsequent dose that is the same as the initial dose or smaller than the initial dose.
317. The method of claim 314 or 315, further comprising determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of the celiac disease for a time period of at least 1 week, at least 2 weeks, at least 3 weeks, at least 4 weeks, at least 6 weeks or at least 8 weeks, if the level of SMAD7, IL-6, and/or TNFα after said administering step is decreased at least 10%, at least 20%, at least 30%, at least 40%, at least 50%, at least 60%, or at least 70% compared to the level of SMAD7, IL-6, and/or TNFα before said administration step.
318. The method of claim 311 or claim 313, wherein if the level of SMAD7, IL-6, and/or TNFα is above normal levels of SMAD7, IL-6, and/or TNFα, then administering to the patient a subsequent dose that is greater than the initial dose, or, if the level of SMAD7, IL-6, and/or TNFα is below normal levels of SMAD7, IL-6, and/or TNFα then administering to the patient a subsequent dose that is smaller than the initial dose.
319. The method of claim 313 or 315, wherein, if the subsequent dose is equal to or greater than the maximum tolerated dose (MTD), then terminating the treatment.
320. The method of claim 313 or 315, wherein the level of SMAD7, IL-6, and/or TNFα is analyzed at least 1 day, at least 3 days, at least 5 days, at least 1 week, at least 2 weeks, at least 3 weeks, at least 1 month, at least 2 months, at least 4 months, or at least 6 months after said administration step.
321. The method of claim 313 or 315, wherein the level of SMAD7, IL-6, and/or TNFα is analyzed immediately after said administration step.
322. The method of claim 313 or 315, wherein the level of SMAD7, IL-6, and/or TNFα is analyzed about 15 days or about 28 days after said administration step.
323. The methods of claim 311 or claim 312, wherein the normal levels of SMAD7, IL-6, and/or TNFα are median levels of SMAD7, IL-6, and/or TNFα in a healthy control group.
324. The methods of claim 315, wherein the control level of SMAD7, IL-6, and/or TNFα is a median level of SMAD7, IL-6, and/or TNFα in a healthy control group.
325. The method of claim 323 or 324, wherein the healthy control group and the patient having celiac disease are matched with respect to age, gender, ethnic origin, smoking habits, dietary habits, body-mass index (BMI), and/or exercise habits.
326. The method of claim 311 or claim 312, wherein the SMAD7, IL-6, and/or TNFα is analyzed by measuring the concentration of SMAD7 protein, SMAD7 mRNA, IL-6 protein, IL-6 mRNA, TNFα protein, and/or TNFα mRNA.
327. The method of claim 315, wherein the SMAD7, IL-6, and/or TNFα is analyzed by measuring the concentration of SMAD7 protein, SMAD7 mRNA, IL-6 protein, IL-6 mRNA, TNFα protein, and/or TNFα mRNA.
328. The method of claim 311, 312, or 315, wherein the initial dose is less than 100 mg/day, less than 90 mg/day, less than 80 mg/day, less than 70 mg/day, less than 60 mg/day, less than 50 mg/day, less than 40 mg/day or less than 30 mg/day.
329. The method of claim 311, 312, or 315, wherein the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day.
330. The method of claim 311, 312, or 315, wherein the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.
331. The method of claim 311, 312, or 315, wherein the initial dose is 10 mg/day, 40 mg/day, 80 mg/day, or 160 mg/day.
332. The method of claim 311 or 313, wherein, if SMAD7, IL-6, and/or TNFα levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.
333. The method of claim 315, wherein, if SMAD7, IL-6, and/or TNFα levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.
334. The method of claim 311 or 313 wherein, if SMAD7, IL-6, and/or TNFα levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.
335. The method of claim 315, wherein, if SMAD7, IL-6, and/or TNFα levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.
336. The method of claim 311, 313, or 315, wherein the initial dose is between about 10 mg/day and 100 mg/day and the subsequent dose is between about 30 mg/day and 200 mg/day.
337. The method of claim 311, 312, or 315, wherein the level of SMAD7, IL-6, and/or TNFα in the patient having celiac disease is determined in a sample obtained from the patient having celiac disease.
338. The method of claim 337, wherein the sample is a blood, serum, plasma, or intestinal tissue sample.
339. The method of claim 311, 312, or 315, wherein the level of SMAD7, IL-6, and/or TNFα is determined by immunochemistry or by nucleotide analysis.
340. The method of claim 339, wherein the level of SMAD7, IL-6, and/or TNFα is determined by an enzyme-linked immunosorbent assay (ELISA).
341. The method of claim 311, 312, or 315, further comprising determining a level of one or more additional analytes in the patient having celiac disease.
342. The method of claim 341, wherein the one or more additional analytes comprise IL-25 and/or IL-15.
343. The method of claim 311, 312, or 315, wherein the specific inhibitor of TNFα is administered orally to the patient having celiac disease.
344. A method for treating or managing celiac disease in a patient with celiac disease having above normal SMAD7, IL-6, and/or TNFα levels following administration of a dose of a specific inhibitor of TNFα, said method comprising administering to said patient a further dose of said specific inhibitor that is greater than or equal to the prior dose.
345. A method for treating or managing celiac disease in a patient with celiac disease having below normal SMAD7, IL-6, and/or TNFα levels following administration of a dose of specific inhibitor of TNFα, said method comprising administering to said patient a further dose of said specific inhibitor that is less than or equal to the prior dose.
346. A method of treating or managing celiac disease in a patient with celiac disease having above normal SMAD7, IL-6, and/or TNFα levels, said method comprising administering to said patient a dose of a specific inhibitor of TNFα.
347. The method of claim 346, wherein the administering is repeated until a SMAD7, IL-6, and/or TNFα level reaches a normal level.
348. A method of monitoring the treatment or management of celiac disease in a patient with celiac disease, the method comprising analyzing SMAD7, IL-6, and/or TNFα levels in the patient following each specific inhibitor of TNFα administration, wherein the absence of an decrease in SMAD7, IL-6, and/or TNFα levels indicates that the treatment or management is not effective.
349. The method of claim 348, wherein SMAD7, IL-6, and/or TNFα levels are analyzed one time, two times, three times, four times, about five times, about 10 times, about 15 times, about 20 times, or about 30 times after each administration of specific inhibitor of TNFα.
350. The method of claim 348, wherein the SMAD7, IL-6, and/or TNFα levels are analyzed immediately after, about 1 hour after, about 3 hours after, about 6 hours after, about 12 hours after, about 1 day after, about 3 days after, about 1 week after, about 2 weeks after, and/or about 1 month after specific inhibitor of TNFα administration.
351. A method of treating or managing celiac disease in a patient with celiac disease having above normal levels of SMAD7, IL-6, and/or TNFα, comprising increasing the amount of a specific inhibitor of TNFα administered to the patient until SMAD7, IL-6, and/or TNFα levels in the patient decrease.
352. The method of claim 351, wherein SMAD7, IL-6, and/or TNFα decreases to about a normal level of SMAD7, IL-6, and/or TNFα or a below normal level of SMAD7, IL-6, and/or TNFα.
353. A specific inhibitor of TNFα for use in a method for treating or managing celiac disease in a patient having celiac disease, wherein the method comprises analyzing the level of SMAD7, IL-6, and/or TNFα in the patient to determine appropriate levels of specific inhibitor of TNFα administration.
354. The specific inhibitor of TNFα for use of claim 353, wherein the method comprises the steps of: (a) administering to the patient an initial dose of the specific inhibitor of TNFα; (b) analyzing the level of a SMAD7, IL-6, and/or TNFα in the patient; and (c) if the level of SMAD7, IL-6, and/or TNFα is above normal levels of SMAD7, IL-6, and/or TNFα, then administering to the patient a subsequent dose of the specific inhibitor of TNFα that is greater than or equal to the initial dose, or, if the level of SMAD7, IL-6, and/or TNFα is below normal levels of SMAD7, IL-6, and/or TNFα then administering to the patient a subsequent dose of the specific inhibitor of TNFα that is equal to or smaller than the initial dose.
355. A specific inhibitor of TNFα for use in a method for treating or managing celiac disease in a patient having celiac disease, wherein the method comprises (a) analyzing the level of SMAD7, IL-6, and/or TNFα in the patient; and (b) if the level of SMAD7, IL-6, and/or TNFα is above normal levels of SMAD7, IL-6, and/or TNFα, then administering to the patient an initial dose of the specific inhibitor of TNFα.
356. The method of any one of claims 311-355, wherein the celiac disease is refractory celiac disease.

### INCORPORATION BY REFERENCE

The entire disclosure of each of the patent documents and scientific articles cited herein is incorporated by reference for all purposes.

### EQUIVALENTS

The invention can be embodied in other specific forms with departing from the essential characteristics thereof. The foregoing embodiments therefore are to be considered illustrative rather than limiting on the invention described herein. The scope of the invention is indicated by the appended claims rather than by the foregoing description, and all changes that come within the meaning and range of equivalency of the claims are intended to be embraced therein.

1. A method of treating, or managing celiac disease or enhancing Transforming Growth Factor-β (TGF-β) signaling in a cell of a patient with celiac disease, comprising inhibiting Mothers Against Decapentaplegic Homolog 7 (SMAD7) in a patient suffering from celiac disease.

2. A method of treating or managing celiac disease or enhancing TGF-β signaling in a cell of a patient with celiac disease, comprising inhibiting SMAD7 in an intestinal cell.

3. A method of treating or managing celiac disease in a patient with celiac disease, comprising administering to the patient an effective amount of a specific inhibitor of SMAD7.

4. The method of claim 2, wherein the intestinal cell is a small intestinal cell.

5. The method of claim 2, wherein the intestinal cell is a large intestinal cell.

6. The method of claim 2, wherein the intestinal cell is a lamina propria mononuclear cell.

7. A method according to any one of the preceding claims, wherein the patient is not suffering from an inflammatory bowel disease.

8. A method according to any one of claims 1-6, wherein celiac disease is preceded by inflammatory bowel disease.

9. A method according to any one of claims 1-6, wherein the patient is suffering from an inflammatory bowel disease.

10. The method of claim 8 or 9, wherein the inflammatory bowel disease is Crohn's disease.

11. The method of claim 8 or 9, wherein the inflammatory bowel disease is ulcerative colitis.

12. The method of claim 3, wherein the inhibitor comprises a SMAD7 antisense oligonucleotide.

13. The method of claim 12, wherein the SMAD7 antisense oligonucleotide comprises an oligonucleotide selected from the group consisting of SEQ ID NO: 2, SEQ ID NO: 3, and SEQ ID NO: 4.

14. The method of claim 12, wherein SMAD7 antisense oligonucleotide comprises SEQ ID NO: 4.

15. The method claim 12, wherein the SMAD7 antisense oligonucleotide is administered parenterally.

16. The method of claim 12, wherein the SMAD7 antisense oligonucleotide is administered orally.

17. The method of claim 3, comprising administering a pharmaceutical composition comprising a SMAD7 antisense oligonucleotide and a pharmaceutically acceptable carrier.

18. The method of claim 17, wherein the SMAD7 antisense oligonucleotide is selected from the group consisting of SEQ ID NO: 2, SEQ ID NO: 3, and SEQ ID NO: 4.

19. The method of claim 18, wherein the SMAD7 antisense oligonucleotide comprises SEQ ID NO: 4.

20. The method of claim 17, wherein the pharmaceutical composition is administered parenterally.

21. The method of claim 17, wherein the pharmaceutical composition is administered orally.

22. The method of claim 17, wherein the pharmaceutical composition comprises an enteric coating comprising an ethylacrylate-methacrylic acid copolymer.

23. The method of claim 1 or 3, wherein the patient is a human.

24. The method of claim 12 or 17, comprising administering at least 100 μg of the antisense oligonucleotide.

25. The method of claim 24, comprising administering from 35 mg to 500 mg of the antisense oligonucleotide.

26. A method for treating or managing celiac disease in a patient having celiac disease, wherein the method comprises (a) administering to the patient an initial dose of a SMAD7 antisense oligonucleotide; (b) analyzing the level of a TGF-β (Transforming Growth Factor-β) signaling activity in the patient; and (c) if the level of TGF-β signaling activity is below normal levels of TGF-β signaling activity, then administering to the patient a subsequent dose that is greater than or equal to the initial dose, or, if the level of TGF-β signaling activity is above normal levels of TGF-β signaling activity, then administering to the patient a subsequent dose that is equal to or smaller than the initial dose.

27. A method for treating or managing celiac disease in a patient having celiac disease, wherein the method comprises (a) analyzing the level of a TGF-β signaling activity in the patient; and (b) if the level of TGF-β signaling activity is below normal levels of TGF-β signaling activity, then administering to the patient an initial dose of a SMAD7 antisense oligonucleotide.

28. The method of claim 27, wherein the method further comprises: (c) analyzing the level of TGF-β signaling activity in the patient after said administering step; and (d) if the level of TGF-β signaling activity is below normal levels of TGF-β signaling activity then administering to the patient a subsequent dose that is greater than or equal to the initial dose, or, if the level of TGF-β signaling activity is above normal levels of TGF-β signaling activity then administering to the patient a subsequent dose that is equal to or smaller than the initial dose.

29. The method of claim 27, wherein the method further comprises: (c) analyzing the level of TGF-β signaling activity in the patient after said administering step; and (d) if the level of TGF-β signaling activity is increased after said administration step than the level of TGF-β signaling activity before said administration step, then administering to the patient a subsequent dose that is the same as the initial dose or smaller than the initial dose, or, if the level of TGF-β signaling activity is unchanged or decreased after said administration step compared to the level of TGF-β signaling activity before said administration step, then administering to the patient a subsequent dose that is the same as the initial dose or greater than the initial dose or terminating the treatment.

30. A method for treating or managing celiac disease in a patient having celiac disease, wherein the method comprises: (a) establishing a control level of a TGF-β signaling activity for the patient; (b) administering to the patient an initial dose of a SMAD7 antisense oligonucleotide; (c) analyzing the level of TGF-β signaling activity in the patient; and (d) if the level of TGF-β signaling activity is higher than the control level, then administering to the patient a subsequent dose that is the same as the initial dose or smaller than the initial dose, or, if the level of TGF-β signaling activity is unchanged or decreased compared to the control level, then administering to the patient a subsequent dose that is the same as the initial dose or greater than the initial dose or terminating the treatment.

31. The method of claim 29 or 30, wherein, if the level of TGF-β signaling activity is at least 10%, at least 20%, at least 30%, at least 40%, at least 50%, or at least 70% increased after said administration step compared to the level of TGF-β signaling activity before said administration step, then administering to the patient a subsequent dose that is the same as the initial dose or smaller than the initial dose.

32. The method of claim 29 or 30, further comprising determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of the celiac disease for a time period of at least 1 week, at least 2 weeks, at least 3 weeks, at least 4 weeks, at least 6 weeks or at least 8 weeks, if the level of TGF-β signaling activity after said administering step is at least 10%, at least 20%, at least 30%, at least 40%, at least 50%, at least 60%, or at least 70% increased compared to the level of TGF-β signaling activity before said administration step.

33. The method of claim 26 or claim 28, wherein if the level of TGF-β signaling activity is below normal levels of TGF-β signaling activity, then administering to the patient a subsequent dose that is greater than the initial dose, or, if the level of TGF-β signaling activity is above normal levels of TGF-β signaling activity then administering to the patient a subsequent dose that is smaller than the initial dose.

34. The method of claim 28 or 30, wherein, if the subsequent dose is equal to or greater than the maximum tolerated dose (MTD), then terminating the treatment.

35. The method of claim 28 or 30, wherein the level of TGF-β signaling activity is analyzed at least 1 day, at least 3 days, at least 5 days, at least 1 week, at least 2 weeks, at least 3 weeks, at least 1 month, at least 2 months, at least 4 months, or at least 6 months after said administration step.

36. The method of claim 28 or 30, wherein the level of TGF-β signaling activity is analyzed immediately after said administration step.

37. The method of claim 28 or 30, wherein the level of TGF-β signaling activity is analyzed about 15 days or about 28 days after said administration step.

38. The methods of claim 26 or claim 27, wherein the normal levels of TGF-β signaling activity are median levels of TGF-β signaling activity in a healthy control group.

39. The methods of claim 30, wherein the control level of TGF-β signaling activity is a median level of TGF-β signaling activity in a healthy control group.

40. The method of claim 38 or 39, wherein the healthy control group and the patient having celiac disease are matched with respect to age, gender, ethnic origin, smoking habits, dietary habits, body-mass index (BMI), and/or exercise habits.

41. The method of claim 26 or claim 27, wherein the TGF-β signaling activity is analyzed by measuring the concentration of a TGF-β signaling analyte.

42. The method of claim 30, wherein the TGF-β signaling activity is analyzed by measuring the concentration of a TGF-β signaling analyte.

43. The method of claim 26, 27, or 30, wherein the initial dose is less than 100 mg/day, less than 90 mg/day, less than 80 mg/day, less than 70 mg/day, less than 60 mg/day, less than 50 mg/day, less than 40 mg/day or less than 30 mg/day.

44. The method of claim 26, 27, or 30, wherein the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day.

45. The method of claim 26, 27, or 30, wherein the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.

46. The method of claim 26, 27, or 30, wherein the initial dose is 10 mg/day, 40 mg/day, 80 mg/day, or 160 mg/day.

47. The method of claim 26 or 28, wherein, if TGF-β signaling activity levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

48. The method of claim 30, wherein, if TGF-β signaling activity levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

49. The method of claim 26 or 28, wherein, if TGF-β signaling activity levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.

50. The method of claim 30, wherein, if TGF-β signaling activity levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.

51. The method of claim 26, 28, or 30, wherein the initial dose is between about 10 mg/day and 100 mg/day and the subsequent dose is between about 30 mg/day and 200 mg/day.

52. The method of claim 26, 27, or 30, wherein the level of TGF-β signaling activity in the patient having celiac disease is determined in a sample obtained from the patient having celiac disease.

53. The method of claim 52, wherein the sample is a blood, serum, plasma, or intestinal tissue sample.

54. The method of claim 26, 27, or 30, wherein the level of TGF-β signaling activity is determined by immunochemistry or by nucleotide analysis.

55. The method of claim 54, wherein the level of TGF-β signaling activity is determined by an enzyme-linked immunosorbent assay (ELISA).

56. The method of claim 26, 27, or 30, further comprising determining a level of one or more additional analytes in the patient having celiac disease.

57. The method of claim 56, wherein the one or more additional analytes comprise Tumor Necrosis Factor α (TNFα), Interleukin-6 (IL-6), Interleukin-25 (IL-25), and/or Interleukin-15 (IL-15).

58. The method of claim 26, 27, or 30, wherein the SMAD7 antisense oligonucleotide is administered orally to the patient having celiac disease.

59. The method of claim 26, 27, or 30, wherein the SMAD7 antisense oligonucleotide targets region 108-128 of human SMAD7 (SEQ ID NO: 1).

60. The method of claim 26, 27, or 30, wherein the SMAD7 antisense oligonucleotide targets nucleotides 403, 233, 294, 295, 296, 298, 299 or 533 of human SMAD7 (SEQ ID NO: 1).

61. The method of claim 26, 27, or 30, wherein the SMAD7 antisense oligonucleotide comprises the nucleotide sequence of SEQ ID NO: 2 (5′-GTCGCCCCTTCTCCCCGCAGC-3′).

62. The method of claim 26, 27, or 30, wherein the antisense oligonucleotide is a SMAD7 phosphorothioate antisense oligonucleotide comprising the following sequence: 5′-GTXGCCCCTTCTCCCXGCAG-3′ (SEQ ID NO: 3) wherein X is a nucleotide comprising 5-methyl-2′-deoxycytidine and wherein the internucleotide linkages are phosphorothioate linkages.

63. The method of claim 62, wherein the antisense oligonucleotide is a SMAD7 phosphorothioate antisense oligonucleotide comprising the following sequence: 5′-GTXGCCCCTTCTCCCXGCAGC-3′ (SEQ ID NO: 4) wherein X is a nucleotide comprising 5-methyl-2′-deoxycytidine and wherein the internucleotide linkages are phosphorothioate linkages.

64. A method for treating or managing celiac disease in a patient with celiac disease having below normal TGF-β signaling activity levels following administration of a dose of a SMAD7 antisense oligonucleotide, said method comprising administering to said patient a further dose of said oligonucleotide that is greater than or equal to the prior dose.

65. A method for treating or managing celiac disease in a patient with celiac disease having above normal TGF-β signaling activity levels following administration of a dose of SMAD7 antisense oligonucleotide, said method comprising administering to said patient a further dose of said oligonucleotide that is less than or equal to the prior dose.

66. A method of treating or managing celiac disease in a patient with celiac disease having below normal TGF-β signaling activity levels, said method comprising administering to said patient a dose of a SMAD7 antisense oligonucleotide.

67. The method of claim 66, wherein the administering is repeated until a TGF-β signaling activity level reaches a normal level.

68. A method of monitoring the treatment or management of celiac disease in a patient with celiac disease, the method comprising analyzing TGF-β signaling activity levels in the patient following each SMAD7 antisense oligonucleotide administration, wherein the absence of an increase in TGF-β signaling activity levels indicates that the treatment or management is not effective.

69. The method of claim 68, wherein TGF-β signaling activity levels are analyzed one time, two times, three times, four times, about five times, about 10 times, about 15 times, about 20 times, or about 30 times after each administration of SMAD7 antisense oligonucleotide.

70. The method of claim 68, wherein the TGF-β signaling activity levels are analyzed immediately after, about 1 hour after, about 3 hours after, about 6 hours after, about 12 hours after, about 1 day after, about 3 days after, about 1 week after, about 2 weeks after, and/or about 1 month after SMAD7 antisense oligonucleotide administration.

71. A method of treating or managing celiac disease in a patient with celiac disease having below normal levels of TGF-β signaling activity, comprising increasing the amount of a SMAD7 antisense oligonucleotide administered to the patient until TGF-β signaling activity levels in the patient increase.

72. The method of claim 71, wherein TGF-β signaling activity increases to about a normal level of TGF-β signaling activity or an above normal level of TGF-β signaling activity.

73. A SMAD7 antisense oligonucleotide for use in a method for treating or managing celiac disease in a patient having celiac disease, wherein the method comprises analyzing the level of TGF-β signaling activity in the patient to determine appropriate levels of SMAD7 antisense oligonucleotide administration.

74. The SMAD7 antisense oligonucleotide for use of claim 73, wherein the method comprises the steps of: (a) administering to the patient an initial dose of the SMAD7 antisense oligonucleotide; (b) analyzing the level of a TGF-β signaling activity in the patient; and (c) if the level of TGF-β signaling activity is below normal levels of TGF-β signaling activity, then administering to the patient a subsequent dose of the SMAD7 antisense oligonucleotide that is greater than or equal to the initial dose, or, if the level of TGF-β signaling activity is above normal levels of TGF-β signaling activity then administering to the patient a subsequent dose of the SMAD7 antisense oligonucleotide that is equal to or smaller than the initial dose.

75. A SMAD7 antisense oligonucleotide for use in a method for treating or managing celiac disease in a patient having celiac disease, wherein the method comprises (a) analyzing the level of TGF-β signaling activity in the patient; and (b) if the level of TGF-β signaling activity is below normal levels of TGF-β signaling activity, then administering to the patient an initial dose of the SMAD7 antisense oligonucleotide.

76. The method of claim 41 or 42, wherein the TGF-β signaling analyte is a protein selected from the group consisting of Transforming Growth Factor-β1 (TGF-β1), Transforming Growth Factor-β2 (TGF-β2), Transforming Growth Factor-β3 (TGF-β3), Mothers Against Decapentaplegic Homolog 2 (SMAD2), Mothers Against Decapentaplegic Homolog 3 (SMAD3), Mothers Against Decapentaplegic Homolog 4 (SMAD4), phosphorylated SMAD2 (p-SMAD2), and phosphorylated SMAD3 (p-SMAD3).

77. The method of any one of claims 1-76, wherein the celiac disease is refractory celiac disease.

78. A method of treating or managing celiac disease or enhancing TGF-β signaling in a cell of a patient with celiac disease, comprising inhibiting IL-6 in a patient suffering from celiac disease.

79. A method of treating or managing celiac disease or enhancing TGF-β signaling in a cell of a patient with celiac disease, comprising inhibiting IL-6 in an intestinal cell.

80. A method of treating or managing celiac disease in a patient with celiac disease, comprising administering to the patient an effective amount of a specific inhibitor of IL-6.

81. The method of claim 78, wherein the intestinal cell is a small intestinal cell.

82. The method of claim 78, wherein the intestinal cell is a large intestinal cell.

83. The method of claim 78, wherein the intestinal cell is a lamina propria mononuclear cell.

84. A method according to any one of claims 78-83, wherein the patient is not suffering from an inflammatory bowel disease.

85. A method according to any one of claims 78-83, wherein celiac disease is preceded by inflammatory bowel disease.

86. A method according to any one of claims 78-83, wherein the patient is suffering from an inflammatory bowel disease.

87. The method of claim 85 or 86, wherein the inflammatory bowel disease is Crohn's disease.

88. The method of claim 85 or 86, wherein the inflammatory bowel disease is ulcerative colitis.

89. The method of claim 80, wherein the inhibitor comprises an IL-6 antisense oligonucleotide.

90. The method claim 89, wherein the IL-6 antisense oligonucleotide is administered parenterally.

91. The method of claim 89, wherein the IL-6 antisense oligonucleotide is administered orally.

92. The method of claim 80, comprising administering a pharmaceutical composition comprising the specific inhibitor of IL-6 and a pharmaceutically acceptable carrier.

93. The method of claim 92, wherein the pharmaceutical composition is administered parenterally.

94. The method of claim 92, wherein the pharmaceutical composition is administered orally.

95. The method of claim 92, wherein the pharmaceutical composition comprises an enteric coating comprising an ethylacrylate-methacrylic acid copolymer.

96. The method of claim 78 or 80, wherein the patient is a human.

97. The method of claim 89, comprising administering at least 100 μg of the antisense oligonucleotide.

98. The method of claim 97, comprising administering from 35 mg to 500 mg of the antisense oligonucleotide.

99. A method for treating or managing celiac disease in a patient having celiac disease, wherein the method comprises (a) administering to the patient an initial dose of specific inhibitor of IL-6; (b) analyzing the level of a TGF-β (Transforming Growth Factor-β) signaling activity in the patient; and (c) if the level of TGF-β signaling activity is below normal levels of TGF-β signaling activity, then administering to the patient a subsequent dose that is greater than or equal to the initial dose, or, if the level of TGF-β signaling activity is above normal levels of TGF-β signaling activity, then administering to the patient a subsequent dose that is equal to or smaller than the initial dose.

100. A method for treating or managing celiac disease in a patient having celiac disease, wherein the method comprises (a) analyzing the level of a TGF-β signaling activity in the patient; and (b) if the level of TGF-β signaling activity is below normal levels of TGF-β signaling activity, then administering to the patient an initial dose of a specific inhibitor of IL-6.

101. The method of claim 100, wherein the method further comprises: (c) analyzing the level of TGF-β signaling activity in the patient after said administering step; and (d) if the level of TGF-β signaling activity is below normal levels of TGF-β signaling activity then administering to the patient a subsequent dose that is greater than or equal to the initial dose, or, if the level of TGF-β signaling activity is above normal levels of TGF-β signaling activity then administering to the patient a subsequent dose that is equal to or smaller than the initial dose.

102. The method of claim 100, wherein the method further comprises: (c) analyzing the level of TGF-β signaling activity in the patient after said administering step; and (d) if the level of TGF-β signaling activity is increased after said administration step than the level of TGF-β signaling activity before said administration step, then administering to the patient a subsequent dose that is the same as the initial dose or smaller than the initial dose, or, if the level of TGF-β signaling activity is unchanged or decreased after said administration step compared to the level of TGF-β signaling activity before said administration step, then administering to the patient a subsequent dose that is the same as the initial dose or greater than the initial dose or terminating the treatment.

103. A method for treating or managing celiac disease in a patient having celiac disease, wherein the method comprises: (a) establishing a control level of a TGF-β signaling activity for the patient; (b) administering to the patient an initial dose of a specific inhibitor of IL-6; (c) analyzing the level of TGF-β signaling activity in the patient; and (d) if the level of TGF-β signaling activity is higher than the control level, then administering to the patient a subsequent dose that is the same as the initial dose or smaller than the initial dose, or, if the level of TGF-β signaling activity is unchanged or decreased compared to the control level, then administering to the patient a subsequent dose that is the same as the initial dose or greater than the initial dose or terminating the treatment.

104. The method of claim 102 or 103, wherein, if the level of TGF-β signaling activity is at least 10%, at least 20%, at least 30%, at least 40%, at least 50%, or at least 70% increased after said administration step compared to the level of TGF-β signaling activity before said administration step, then administering to the patient a subsequent dose that is the same as the initial dose or smaller than the initial dose.

105. The method of claim 102 or 103, further comprising determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of the celiac disease for a time period of at least 1 week, at least 2 weeks, at least 3 weeks, at least 4 weeks, at least 6 weeks or at least 8 weeks, if the level of TGF-β signaling activity after said administering step is at least 10%, at least 20%, at least 30%, at least 40%, at least 50%, at least 60%, or at least 70% increased compared to the level of TGF-β signaling activity before said administration step.

106. The method of claim 99 or claim 101, wherein if the level of TGF-β signaling activity is below normal levels of TGF-β signaling activity, then administering to the patient a subsequent dose that is greater than the initial dose, or, if the level of TGF-β signaling activity is above normal levels of TGF-β signaling activity then administering to the patient a subsequent dose that is smaller than the initial dose.

107. The method of claim 101 or 103, wherein, if the subsequent dose is equal to or greater than the maximum tolerated dose (MTD), then terminating the treatment.

108. The method of claim 101 or 103, wherein the level of TGF-β signaling activity is analyzed at least 1 day, at least 3 days, at least 5 days, at least 1 week, at least 2 weeks, at least 3 weeks, at least 1 month, at least 2 months, at least 4 months, or at least 6 months after said administration step.

109. The method of claim 101 or 103, wherein the level of TGF-β signaling activity is analyzed immediately after said administration step.

110. The method of claim 101 or 103, wherein the level of TGF-β signaling activity is analyzed about 15 days or about 28 days after said administration step.

111. The methods of claim 99 or claim 100, wherein the normal levels of TGF-β signaling activity are median levels of TGF-β signaling activity in a healthy control group.

112. The methods of claim 103, wherein the control level of TGF-β signaling activity is a median level of TGF-β signaling activity in a healthy control group.

113. The method of claim 111 or 112, wherein the healthy control group and the patient having celiac disease are matched with respect to age, gender, ethnic origin, smoking habits, dietary habits, body-mass index (BMI), and/or exercise habits.

114. The method of claim 99 or claim 100, wherein the TGF-β signaling activity is analyzed by measuring the concentration of a TGF-β signaling analyte.

115. The method of claim 103, wherein the TGF-β signaling activity is analyzed by measuring the concentration of a TGF-β signaling analyte.

116. The method of claim 99, 100, or 103, wherein the initial dose is less than 100 mg/day, less than 90 mg/day, less than 80 mg/day, less than 70 mg/day, less than 60 mg/day, less than 50 mg/day, less than 40 mg/day or less than 30 mg/day.

117. The method of claim 99, 100, or 103, wherein the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day.

118. The method of claim 99, 100, or 103, wherein the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.

119. The method of claim 99, 100, or 103, wherein the initial dose is 10 mg/day, 40 mg/day, 80 mg/day, or 160 mg/day.

120. The method of claim 99 or 101, wherein, if TGF-β signaling activity levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

121. The method of claim 103, wherein, if TGF-β signaling activity levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

122. The method of claim 99 or 101, wherein, if TGF-β signaling activity levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.

123. The method of claim 103, wherein, if TGF-β signaling activity levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.

124. The method of claim 99, 101, or 103, wherein the initial dose is between about 10 mg/day and 100 mg/day and the subsequent dose is between about 30 mg/day and 200 mg/day.

125. The method of claim 99, 100, or 103, wherein the level of TGF-β signaling activity in the patient having celiac disease is determined in a sample obtained from the patient having celiac disease.

126. The method of claim 125, wherein the sample is a blood, serum, plasma, or intestinal tissue sample.

127. The method of claim 99, 100, or 103, wherein the level of TGF-β signaling activity is determined by immunochemistry or by nucleotide analysis.

128. The method of claim 127, wherein the level of TGF-β signaling activity is determined by an enzyme-linked immunosorbent assay (ELISA).

129. The method of claim 99, 100, or 103, further comprising determining a level of one or more additional analytes in the patient having celiac disease.

130. The method of claim 129, wherein the one or more additional analytes comprise TNFα, SMAD7, IL-25, and/or IL-15.

131. The method of claim 99, 100, or 103, wherein the specific inhibitor of IL-6 is administered orally to the patient having celiac disease.

132. A method for treating or managing celiac disease in a patient with celiac disease having below normal TGF-β signaling activity levels following administration of a dose of a specific inhibitor of IL-6, said method comprising administering to said patient a further dose of said specific inhibitor of IL-6 that is greater than or equal to the prior dose.

133. A method for treating or managing celiac disease in a patient with celiac disease having above normal TGF-β signaling activity levels following administration of a dose of a specific inhibitor of IL-6, said method comprising administering to said patient a further dose of said specific inhibitor of IL-6 that is less than or equal to the prior dose.

134. A method of treating or managing celiac disease in a patient with celiac disease having below normal TGF-β signaling activity levels, said method comprising administering to said patient a dose of a specific inhibitor of IL-6.

135. The method of claim 134, wherein the administering is repeated until a TGF-β signaling activity level reaches a normal level.

136. A method of monitoring the treatment or management of celiac disease in a patient with celiac disease, the method comprising analyzing TGF-β signaling activity levels in the patient following each specific inhibitor of IL-6 administration, wherein the absence of an increase in TGF-β signaling activity levels indicates that the treatment or management is not effective.

137. The method of claim 136, wherein TGF-β signaling activity levels are analyzed one time, two times, three times, four times, about five times, about 10 times, about 15 times, about 20 times, or about 30 times after each administration of the specific inhibitor of IL-6.

138. The method of claim 136, wherein the TGF-β signaling activity levels are analyzed immediately after, about 1 hour after, about 3 hours after, about 6 hours after, about 12 hours after, about 1 day after, about 3 days after, about 1 week after, about 2 weeks after, and/or about 1 month after administration of the specific inhibitor of IL-6.

139. A method of treating or managing celiac disease in a patient with celiac disease having below normal levels of TGF-β signaling activity, comprising increasing the amount of a specific inhibitor of IL-6 administered to the patient until TGF-β signaling activity levels in the patient increase.

140. The method of claim 139, wherein TGF-β signaling activity increases to about a normal level of TGF-β signaling activity or an above normal level of TGF-β signaling activity.

141. A specific inhibitor of IL-6 for use in a method for treating or managing celiac disease in a patient having celiac disease, wherein the method comprises analyzing the level of TGF-β signaling activity in the patient to determine appropriate levels of specific inhibitor of IL-6 administration.

142. The specific inhibitor of IL-6 for use of claim 141, wherein the method comprises the steps of: (a) administering to the patient an initial dose of the specific inhibitor of IL-6; (b) analyzing the level of a TGF-β signaling activity in the patient; and (c) if the level of TGF-β signaling activity is below normal levels of TGF-β signaling activity, then administering to the patient a subsequent dose of the specific inhibitor of IL-6 that is greater than or equal to the initial dose, or, if the level of TGF-β signaling activity is above normal levels of TGF-β signaling activity then administering to the patient a subsequent dose of the specific inhibitor of IL-6 that is equal to or smaller than the initial dose.

143. A specific inhibitor of IL-6 for use in a method for treating or managing celiac disease in a patient having celiac disease, wherein the method comprises (a) analyzing the level of TGF-β signaling activity in the patient; and (b) if the level of TGF-β signaling activity is below normal levels of TGF-β signaling activity, then administering to the patient an initial dose of the specific inhibitor of IL-6.

144. The method of claim 114 or 115, wherein the TGF-β signaling analyte is a protein selected from the group consisting of TGF-β1, TGF-β2, TGF-β3, SMAD2, SMAD3, SMAD4, p-SMAD2, and p-SMAD3.

145. The method of any one of claims 78-144, wherein the celiac disease is refractory celiac disease.

146. A method of treating or managing celiac disease or enhancing TGF-β signaling in a cell of a patient with celiac disease, comprising inhibiting TNFα in a patient suffering from celiac disease.

147. A method of treating or managing celiac disease or enhancing TGF-β signaling in a cell of a patient with celiac disease, comprising inhibiting TNFα in an intestinal cell.

148. A method of treating or managing celiac disease in a patient with celiac disease, comprising administering to the patient an effective amount of a specific inhibitor of TNFα.

149. The method of claim 146, wherein the intestinal cell is a small intestinal cell.

150. The method of claim 146, wherein the intestinal cell is a large intestinal cell.

151. The method of claim 146, wherein the intestinal cell is a lamina propria mononuclear cell.

152. A method according to any one of claims 146-151, wherein the patient is not suffering from an inflammatory bowel disease.

153. A method according to any one of claims 146-151, wherein celiac disease is preceded by inflammatory bowel disease.

154. A method according to any one of claims 146-151, wherein the patient is suffering from an inflammatory bowel disease.

155. The method of claim 153 or 154, wherein the inflammatory bowel disease is Crohn's disease.

156. The method of claim 153 or 154, wherein the inflammatory bowel disease is ulcerative colitis.

157. The method of claim 148, wherein the inhibitor comprises a TNFα antisense oligonucleotide.

158. The method claim 157, wherein the TNFα antisense oligonucleotide is administered parenterally.

159. The method of claim 157, wherein TNFα antisense oligonucleotide is administered orally.

160. The method of claim 148, comprising administering a pharmaceutical composition comprising a specific inhibitor of TNFα and a pharmaceutically acceptable carrier.

161. The method of claim 160, wherein the pharmaceutical composition is administered parenterally.

162. The method of claim 160, wherein the pharmaceutical composition is administered orally.

163. The method of claim 160, wherein the pharmaceutical composition comprises an enteric coating comprising an ethylacrylate-methacrylic acid copolymer.

164. The method of claim 146 or 148, wherein the patient is a human.

165. The method of claim 157, comprising administering at least 100 μg of the antisense oligonucleotide.

166. The method of claim 165, comprising administering from 35 mg to 500 mg of the antisense oligonucleotide.

167. A method for treating or managing celiac disease in a patient having celiac disease, wherein the method comprises (a) administering to the patient an initial dose of specific inhibitor of TNFα; (b) analyzing the level of a TGF-β (Transforming Growth Factor-β) signaling activity in the patient; and (c) if the level of TGF-β signaling activity is below normal levels of TGF-β signaling activity, then administering to the patient a subsequent dose that is greater than or equal to the initial dose, or, if the level of TGF-β signaling activity is above normal levels of TGF-β signaling activity, then administering to the patient a subsequent dose that is equal to or smaller than the initial dose.

168. A method for treating or managing celiac disease in a patient having celiac disease, wherein the method comprises (a) analyzing the level of a TGF-β signaling activity in the patient; and (b) if the level of TGF-β signaling activity is below normal levels of TGF-β signaling activity, then administering to the patient an initial dose of a specific inhibitor of TNFα.

169. The method of claim 168, wherein the method further comprises: (c) analyzing the level of TGF-β signaling activity in the patient after said administering step; and (d) if the level of TGF-β signaling activity is below normal levels of TGF-β signaling activity then administering to the patient a subsequent dose that is greater than or equal to the initial dose, or, if the level of TGF-β signaling activity is above normal levels of TGF-β signaling activity then administering to the patient a subsequent dose that is equal to or smaller than the initial dose.

170. The method of claim 168, wherein the method further comprises: (c) analyzing the level of TGF-β signaling activity in the patient after said administering step; and (d) if the level of TGF-β signaling activity is increased after said administration step than the level of TGF-β signaling activity before said administration step, then administering to the patient a subsequent dose that is the same as the initial dose or smaller than the initial dose, or, if the level of TGF-β signaling activity is unchanged or decreased after said administration step compared to the level of TGF-β signaling activity before said administration step, then administering to the patient a subsequent dose that is the same as the initial dose or greater than the initial dose or terminating the treatment.

171. A method for treating or managing celiac disease in a patient having celiac disease, wherein the method comprises: (a) establishing a control level of a TGF-β signaling activity for the patient; (b) administering to the patient an initial dose of a specific inhibitor of TNFα; (c) analyzing the level of TGF-β signaling activity in the patient; and (d) if the level of TGF-β signaling activity is higher than the control level, then administering to the patient a subsequent dose that is the same as the initial dose or smaller than the initial dose, or, if the level of TGF-β signaling activity is unchanged or decreased compared to the control level, then administering to the patient a subsequent dose that is the same as the initial dose or greater than the initial dose or terminating the treatment.

172. The method of claim 170 or 171, wherein, if the level of TGF-β signaling activity is at least 10%, at least 20%, at least 30%, at least 40%, at least 50%, or at least 70% increased after said administration step compared to the level of TGF-β signaling activity before said administration step, then administering to the patient a subsequent dose that is the same as the initial dose or smaller than the initial dose.

173. The method of claim 170 or 171, further comprising determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of the celiac disease for a time period of at least 1 week, at least 2 weeks, at least 3 weeks, at least 4 weeks, at least 6 weeks or at least 8 weeks, if the level of TGF-β signaling activity after said administering step is at least 10%, at least 20%, at least 30%, at least 40%, at least 50%, at least 60%, or at least 70% increased compared to the level of TGF-β signaling activity before said administration step.

174. The method of claim 167 or claim 169, wherein if the level of TGF-β signaling activity is below normal levels of TGF-β signaling activity, then administering to the patient a subsequent dose that is greater than the initial dose, or, if the level of TGF-β signaling activity is above normal levels of TGF-β signaling activity then administering to the patient a subsequent dose that is smaller than the initial dose.

175. The method of claim 169 or 171, wherein, if the subsequent dose is equal to or greater than the maximum tolerated dose (MTD), then terminating the treatment.

176. The method of claim 169 or 171, wherein the level of TGF-β signaling activity is analyzed at least 1 day, at least 3 days, at least 5 days, at least 1 week, at least 2 weeks, at least 3 weeks, at least 1 month, at least 2 months, at least 4 months, or at least 6 months after said administration step.

177. The method of claim 169 or 171, wherein the level of TGF-β signaling activity is analyzed immediately after said administration step.

178. The method of claim 169 or 171, wherein the level of TGF-β signaling activity is analyzed about 15 days or about 28 days after said administration step.

179. The methods of claim 173 or claim 174, wherein the normal levels of TGF-β signaling activity are median levels of TGF-β signaling activity in a healthy control group.

180. The methods of claim 171, wherein the control level of TGF-β signaling activity is a median level of TGF-β signaling activity in a healthy control group.

181. The method of claim 179 or 180, wherein the healthy control group and the patient having celiac disease are matched with respect to age, gender, ethnic origin, smoking habits, dietary habits, body-mass index (BMI), and/or exercise habits.

182. The method of claim 167 or claim 168, wherein the TGF-β signaling activity is analyzed by measuring the concentration of a TGF-β signaling analyte.

183. The method of claim 171, wherein the TGF-β signaling activity is analyzed by measuring the concentration of a TGF-β signaling analyte.

184. The method of claim 167, 168, or 171, wherein the initial dose is less than 100 mg/day, less than 90 mg/day, less than 80 mg/day, less than 70 mg/day, less than 60 mg/day, less than 50 mg/day, less than 40 mg/day or less than 30 mg/day.

185. The method of claim 167, 168, or 171, wherein the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day.

186. The method of claim 167, 168, or 171, wherein the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.

187. The method of claim 167, 168, or 171, wherein the initial dose is 10 mg/day, 40 mg/day, 80 mg/day, or 160 mg/day.

188. The method of claim 167 or 169, wherein, if TGF-β signaling activity levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

189. The method of claim 171, wherein, if TGF-β signaling activity levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

190. The method of claim 167 or 169, wherein, if TGF-β signaling activity levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.

191. The method of claim 171, wherein, if TGF-β signaling activity levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.

192. The method of claim 167, 169, or 171, wherein the initial dose is between about 10 mg/day and 100 mg/day and the subsequent dose is between about 30 mg/day and 200 mg/day.

193. The method of claim 167, 168, or 171, wherein the level of TGF-β signaling activity in the patient having celiac disease is determined in a sample obtained from the patient having celiac disease.

194. The method of claim 193, wherein the sample is a blood, serum, plasma, or intestinal tissue sample.

195. The method of claim 167, 168, or 171, wherein the level of TGF-β signaling activity is determined by immunochemistry or by nucleotide analysis.

196. The method of claim 195, wherein the level of TGF-β signaling activity is determined by an enzyme-linked immunosorbent assay (ELISA).

197. The method of claim 167, 168, or 171, further comprising determining a level of one or more additional analytes in the patient having celiac disease.

198. The method of claim 197, wherein the one or more additional analytes comprise IL-6, SMAD7, IL-25, and/or IL-15.

199. The method of claim 167, 168, or 171, wherein the specific inhibitor of TNFα is administered orally to the patient having celiac disease.

200. A method for treating or managing celiac disease in a patient with celiac disease having below normal TGF-β signaling activity levels following administration of a dose of a specific inhibitor of TNFα, said method comprising administering to said patient a further dose of said specific inhibitor of TNFα that is greater than or equal to the prior dose.

201. A method for treating or managing celiac disease in a patient with celiac disease having above normal TGF-β signaling activity levels following administration of a dose of a specific inhibitor of TNFα, said method comprising administering to said patient a further dose of said specific inhibitor of TNFα that is less than or equal to the prior dose.

202. A method of treating or managing celiac disease in a patient with celiac disease having below normal TGF-β signaling activity levels, said method comprising administering to said patient a dose of a specific inhibitor of TNFα.

203. The method of claim 202, wherein the administering is repeated until a TGF-β signaling activity level reaches a normal level.

204. A method of monitoring the treatment or management of celiac disease in a patient with celiac disease, the method comprising analyzing TGF-β signaling activity levels in the patient following each specific inhibitor of TNFα administration, wherein the absence of an increase in TGF-β signaling activity levels indicates that the treatment or management is not effective.

205. The method of claim 204, wherein TGF-β signaling activity levels are analyzed one time, two times, three times, four times, about five times, about 10 times, about 15 times, about 20 times, or about 30 times after each administration of the specific inhibitor of TNFα.

206. The method of claim 204, wherein the TGF-β signaling activity levels are analyzed immediately after, about 1 hour after, about 3 hours after, about 6 hours after, about 12 hours after, about 1 day after, about 3 days after, about 1 week after, about 2 weeks after, and/or about 1 month after administration of the specific inhibitor of TNFα.

207. A method of treating or managing celiac disease in a patient with celiac disease having below normal levels of TGF-β signaling activity, comprising increasing the amount of a specific inhibitor of TNFα administered to the patient until TGF-β signaling activity levels in the patient increase.

208. The method of claim 207, wherein TGF-β signaling activity increases to about a normal level of TGF-β signaling activity or an above normal level of TGF-β signaling activity.

209. A specific inhibitor of TNFα for use in a method for treating or managing celiac disease in a patient having celiac disease, wherein the method comprises analyzing the level of TGF-β signaling activity in the patient to determine appropriate levels of specific inhibitor of TNFα administration.

210. The specific inhibitor of TNFα for use of claim 209, wherein the method comprises the steps of: (a) administering to the patient an initial dose of the specific inhibitor of TNFα; (b) analyzing the level of a TGF-β signaling activity in the patient; and (c) if the level of TGF-β signaling activity is below normal levels of TGF-β signaling activity, then administering to the patient a subsequent dose of the specific inhibitor of TNFα that is greater than or equal to the initial dose, or, if the level of TGF-β signaling activity is above normal levels of TGF-β signaling activity then administering to the patient a subsequent dose of the specific inhibitor of TNFα that is equal to or smaller than the initial dose.

211. A specific inhibitor of TNFα for use in a method for treating or managing celiac disease in a patient having celiac disease, wherein the method comprises (a) analyzing the level of TGF-β signaling activity in the patient; and (b) if the level of TGF-β signaling activity is below normal levels of TGF-β signaling activity, then administering to the patient an initial dose of the specific inhibitor of TNFα.

212. The method of claim 182 or 183, wherein the TGF-β signaling analyte is a protein selected from the group consisting of TGF-β1, TGF-β2, TGF-β3, SMAD2, SMAD3, SMAD4, p-SMAD2, and p-SMAD3.

213. The method of any one of claims 146-212, wherein the celiac disease is refractory celiac disease.

214. A method for treating or managing celiac disease in a patient having celiac disease, wherein the method comprises (a) administering to the patient an initial dose of a SMAD7 antisense oligonucleotide; (b) analyzing the level of SMAD7, IL-6, and/or TNFα in the patient; and (c) if the level of SMAD7, IL-6, and/or TNFα is above normal levels of SMAD7, IL-6, and/or TNFα, then administering to the patient a subsequent dose that is greater than or equal to the initial dose, or, if the level SMAD7, IL-6, and/or TNFα is below normal levels of SMAD7, IL-6, and/or TNFα, then administering to the patient a subsequent dose that is equal to or smaller than the initial dose.

215. A method for treating or managing celiac disease in a patient having celiac disease, wherein the method comprises (a) analyzing the level of a SMAD7, IL-6, and/or TNFα in the patient; and (b) if the level of SMAD7, IL-6, and/or TNFα is above normal levels of SMAD7, IL-6, and/or TNFα, then administering to the patient an initial dose of a SMAD7 antisense oligonucleotide.

216. The method of claim 215, wherein the method further comprises: (c) analyzing the level of SMAD7, IL-6, and/or TNFα in the patient after said administering step; and (d) if the level of SMAD7, IL-6, and/or TNFα is above normal levels of SMAD7, IL-6, and/or TNFα then administering to the patient a subsequent dose that is greater than or equal to the initial dose, or, if the level of SMAD7, IL-6, and/or TNFα is below normal levels of SMAD7, IL-6, and/or TNFα then administering to the patient a subsequent dose that is equal to or smaller than the initial dose.

217. The method of claim 215, wherein the method further comprises: (c) analyzing the level of SMAD7, IL-6, and/or TNFα in the patient after said administering step; and (d) if the level of SMAD7, IL-6, and/or TNFα is decreased after said administration step compared to the level of SMAD7, IL-6, and/or TNFα before said administration step, then administering to the patient a subsequent dose that is the same as the initial dose or smaller than the initial dose, or, if the level of SMAD7, IL-6, and/or TNFα is unchanged or increased after said administration step compared to the level of SMAD7, IL-6, and/or TNFα before said administration step, then administering to the patient a subsequent dose that is the same as the initial dose or greater than the initial dose or terminating the treatment.

218. A method for treating or managing celiac disease in a patient having celiac disease, wherein the method comprises: (a) establishing a control level of a SMAD7, IL-6, and/or TNFα for the patient; (b) administering to the patient an initial dose of a SMAD7 antisense oligonucleotide; (c) analyzing the level of SMAD7, IL-6, and/or TNFα in the patient; and (d) if the level of SMAD7, IL-6, and/or TNFα is lower than the control level, then administering to the patient a subsequent dose that is the same as the initial dose or smaller than the initial dose, or, if the level of SMAD7, IL-6, and/or TNFα is unchanged or increased compared to the control level, then administering to the patient a subsequent dose that is the same as the initial dose or greater than the initial dose or terminating the treatment.

219. The method of claim 217 or 218, wherein, if the level of SMAD7, IL-6, and/or TNFα is at least 10%, at least 20%, at least 30%, at least 40%, at least 50%, or at least 70% decreased after said administration step compared to the level of SMAD7, IL-6, and/or TNFα before said administration step, then administering to the patient a subsequent dose that is the same as the initial dose or smaller than the initial dose.

220. The method of claim 217 or 218, further comprising determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of the celiac disease for a time period of at least 1 week, at least 2 weeks, at least 3 weeks, at least 4 weeks, at least 6 weeks or at least 8 weeks, if the level of SMAD7, IL-6, and/or TNFα after said administering step is decreased at least 10%, at least 20%, at least 30%, at least 40%, at least 50%, at least 60%, or at least 70% compared to the level of SMAD7, IL-6, and/or TNFα before said administration step.

221. The method of claim 214 or claim 216, wherein if the level of SMAD7, IL-6, and/or TNFα is above normal levels of SMAD7, IL-6, and/or TNFα, then administering to the patient a subsequent dose that is greater than the initial dose, or, if the level of SMAD7, IL-6, and/or TNFα is below normal levels of SMAD7, IL-6, and/or TNFα then administering to the patient a subsequent dose that is smaller than the initial dose.

222. The method of claim 216 or 218, wherein, if the subsequent dose is equal to or greater than the maximum tolerated dose (MTD), then terminating the treatment.

223. The method of claim 216 or 218, wherein the level of SMAD7, IL-6, and/or TNFα is analyzed at least 1 day, at least 3 days, at least 5 days, at least 1 week, at least 2 weeks, at least 3 weeks, at least 1 month, at least 2 months, at least 4 months, or at least 6 months after said administration step.

224. The method of claim 216 or 218, wherein the level of SMAD7, IL-6, and/or TNFα is analyzed immediately after said administration step.

225. The method of claim 216 or 218, wherein the level of SMAD7, IL-6, and/or TNFα is analyzed about 15 days or about 28 days after said administration step.

226. The methods of claim 214 or claim 215, wherein the normal levels of SMAD7, IL-6, and/or TNFα are median levels of SMAD7, IL-6, and/or TNFα in a healthy control group.

227. The methods of claim 218, wherein the control level of SMAD7, IL-6, and/or TNFα is a median level of SMAD7, IL-6, and/or TNFα in a healthy control group.

228. The method of claim 226 or 227, wherein the healthy control group and the patient having celiac disease are matched with respect to age, gender, ethnic origin, smoking habits, dietary habits, body-mass index (BMI), and/or exercise habits.

229. The method of claim 214 or claim 215, wherein the SMAD7, IL-6, and/or TNFα is analyzed by measuring the concentration of SMAD7 protein, SMAD7 mRNA, IL-6 protein, IL-6 mRNA, TNFα protein, and/or TNFα mRNA.

230. The method of claim 218, wherein the SMAD7, IL-6, and/or TNFα is analyzed by measuring the concentration of SMAD7 protein, SMAD7 mRNA, IL-6 protein, IL-6 mRNA, TNFα protein, and/or TNFα mRNA.

231. The method of claim 214, 215, or 218, wherein the initial dose is less than 100 mg/day, less than 90 mg/day, less than 80 mg/day, less than 70 mg/day, less than 60 mg/day, less than 50 mg/day, less than 40 mg/day or less than 30 mg/day.

232. The method of claim 214, 215, or 218, wherein the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day.

233. The method of claim 214, 215, or 218, wherein the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.

234. The method of claim 214, 215, or 218, wherein the initial dose is 10 mg/day, 40 mg/day, 80 mg/day, or 160 mg/day.

235. The method of claim 214 or 216, wherein, if SMAD7, IL-6, and/or TNFα levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

236. The method of claim 218, wherein, if SMAD7, IL-6, and/or TNFα levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

237. The method of claim 214 or 16 wherein, if SMAD7, IL-6, and/or TNFα levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.

238. The method of claim 218, wherein, if SMAD7, IL-6, and/or TNFα levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.

239. The method of claim 214, 216, or 218, wherein the initial dose is between about 10 mg/day and 100 mg/day and the subsequent dose is between about 30 mg/day and 200 mg/day.

240. The method of claim 214, 215, or 218, wherein the level of SMAD7, IL-6, and/or TNFα in the patient having celiac disease is determined in a sample obtained from the patient having celiac disease.

241. The method of claim 240, wherein the sample is a blood, serum, plasma, or intestinal tissue sample.

242. The method of claim 214, 215, or 218, wherein the level of SMAD7, IL-6, and/or TNFα is determined by immunochemistry or by nucleotide analysis.

243. The method of claim 242, wherein the level of SMAD7, IL-6, and/or TNFα is determined by an enzyme-linked immunosorbent assay (ELISA).

244. The method of claim 214, 215, or 218, further comprising determining a level of one or more additional analytes in the patient having celiac disease.

245. The method of claim 244, wherein the one or more additional analytes comprise IL-25 and/or IL-15.

246. The method of claim 214, 215, or 218, wherein the SMAD7 antisense oligonucleotide is administered orally to the patient having celiac disease.

247. The method of claim 214, 215, or 218, wherein the SMAD7 antisense oligonucleotide targets region 108-128 of human SMAD7 (SEQ ID NO: 1).

248. The method of claim 214, 215, or 218, wherein the SMAD7 antisense oligonucleotide targets nucleotides 403, 233, 294, 295, 296, 298, 299 or 533 of human SMAD7 (SEQ ID NO: 1).

249. The method of claim 214, 215, or 218, wherein the SMAD7 antisense oligonucleotide comprises the nucleotide sequence of SEQ ID NO: 2 (5′-GTCGCCCCTTCTCCCCGCAGC-3′).

250. The method of claim 214, 215, or 218, wherein the antisense oligonucleotide is a SMAD7 phosphorothioate antisense oligonucleotide comprising the following sequence: 5′-GTXGCCCCTTCTCCCXGCAG-3′ (SEQ ID NO: 3) wherein X is a nucleotide comprising 5-methyl-2′-deoxycytidine and wherein the internucleotide linkages are phosphorothioate linkages.

251. The method of claim 250, wherein the antisense oligonucleotide is a SMAD7 phosphorothioate antisense oligonucleotide comprising the following sequence: 5′-GTXGCCCCTTCTCCCXGCAGC-3′ (SEQ ID NO: 4) wherein X is a nucleotide comprising 5-methyl-2′-deoxycytidine and wherein the internucleotide linkages are phosphorothioate linkages.

252. A method for treating or managing celiac disease in a patient with celiac disease having above normal SMAD7, IL-6, and/or TNFα levels following administration of a dose of a SMAD7 antisense oligonucleotide, said method comprising administering to said patient a further dose of said oligonucleotide that is greater than or equal to the prior dose.

253. A method for treating or managing celiac disease in a patient with celiac disease having below normal SMAD7, IL-6, and/or TNFα levels following administration of a dose of SMAD7 antisense oligonucleotide, said method comprising administering to said patient a further dose of said oligonucleotide that is less than or equal to the prior dose.

254. A method of treating or managing celiac disease in a patient with celiac disease having above normal SMAD7, IL-6, and/or TNFα levels, said method comprising administering to said patient a dose of a SMAD7 antisense oligonucleotide.

255. The method of claim 254, wherein the administering is repeated until a SMAD7, IL-6, and/or TNFα level reaches a normal level.

256. A method of monitoring the treatment or management of celiac disease in a patient with celiac disease, the method comprising analyzing SMAD7, IL-6, and/or TNFα levels in the patient following each SMAD7 antisense oligonucleotide administration, wherein the absence of an decrease in SMAD7, IL-6, and/or TNFα levels indicates that the treatment or management is not effective.

257. The method of claim 256, wherein SMAD7, IL-6, and/or TNFα levels are analyzed one time, two times, three times, four times, about five times, about 10 times, about 15 times, about 20 times, or about 30 times after each administration of SMAD7 antisense oligonucleotide.

258. The method of claim 256, wherein the SMAD7, IL-6, and/or TNFα levels are analyzed immediately after, about 1 hour after, about 3 hours after, about 6 hours after, about 12 hours after, about 1 day after, about 3 days after, about 1 week after, about 2 weeks after, and/or about 1 month after SMAD7 antisense oligonucleotide administration.

259. A method of treating or managing celiac disease in a patient with celiac disease having above normal levels of SMAD7, IL-6, and/or TNFα, comprising increasing the amount of a SMAD7 antisense oligonucleotide administered to the patient until SMAD7, IL-6, and/or TNFα levels in the patient decrease.

260. The method of claim 259, wherein SMAD7, IL-6, and/or TNFα decreases to about a normal level of SMAD7, IL-6, and/or TNFα or a below normal level of SMAD7, IL-6, and/or TNFα.

261. A SMAD7 antisense oligonucleotide for use in a method for treating or managing celiac disease in a patient having celiac disease, wherein the method comprises analyzing the level of SMAD7, IL-6, and/or TNFα in the patient to determine appropriate levels of SMAD7 antisense oligonucleotide administration.

262. The SMAD7 antisense oligonucleotide for use of claim 261, wherein the method comprises the steps of: (a) administering to the patient an initial dose of the SMAD7 antisense oligonucleotide; (b) analyzing the level of a SMAD7, IL-6, and/or TNFα in the patient; and (c) if the level of SMAD7, IL-6, and/or TNFα is above normal levels of SMAD7, IL-6, and/or TNFα, then administering to the patient a subsequent dose of the SMAD7 antisense oligonucleotide that is greater than or equal to the initial dose, or, if the level of SMAD7, IL-6, and/or TNFα is below normal levels of SMAD7, IL-6, and/or TNFα then administering to the patient a subsequent dose of the SMAD7 antisense oligonucleotide that is equal to or smaller than the initial dose.

263. A SMAD7 antisense oligonucleotide for use in a method for treating or managing celiac disease in a patient having celiac disease, wherein the method comprises (a) analyzing the level of SMAD7, IL-6, and/or TNFα in the patient; and (b) if the level of SMAD7, IL-6, and/or TNFα is above normal levels of SMAD7, IL-6, and/or TNFα, then administering to the patient an initial dose of the SMAD7 antisense oligonucleotide.

264. The method of any one of claims 214-260 or the antisense oligonucleotide of any one of claims 261-263, wherein the celiac disease is refractory celiac disease.

265. A method for treating or managing celiac disease in a patient having celiac disease, wherein the method comprises (a) administering to the patient an initial dose of a specific inhibitor of IL-6; (b) analyzing the level of SMAD7, IL-6, and/or TNFα in the patient; and (c) if the level of SMAD7, IL-6, and/or TNFα is above normal levels of SMAD7, IL-6, and/or TNFα, then administering to the patient a subsequent dose that is greater than or equal to the initial dose, or, if the level SMAD7, IL-6, and/or TNFα is below normal levels of SMAD7, IL-6, and/or TNFα, then administering to the patient a subsequent dose that is equal to or smaller than the initial dose.

266. A method for treating or managing celiac disease in a patient having celiac disease, wherein the method comprises (a) analyzing the level of a SMAD7, IL-6, and/or TNFα in the patient; and (b) if the level of SMAD7, IL-6, and/or TNFα is above normal levels of SMAD7, IL-6, and/or TNFα, then administering to the patient an initial dose of a specific inhibitor of IL-6.

267. The method of claim 266, wherein the method further comprises: (c) analyzing the level of SMAD7, IL-6, and/or TNFα in the patient after said administering step; and (d) if the level of SMAD7, IL-6, and/or TNFα is above normal levels of SMAD7, IL-6, and/or TNFα then administering to the patient a subsequent dose that is greater than or equal to the initial dose, or, if the level of SMAD7, IL-6, and/or TNFα is below normal levels of SMAD7, IL-6, and/or TNFα then administering to the patient a subsequent dose that is equal to or smaller than the initial dose.

268. The method of claim 266, wherein the method further comprises: (c) analyzing the level of SMAD7, IL-6, and/or TNFα in the patient after said administering step; and (d) if the level of SMAD7, IL-6, and/or TNFα is decreased after said administration step compared to the level of SMAD7, IL-6, and/or TNFα before said administration step, then administering to the patient a subsequent dose that is the same as the initial dose or smaller than the initial dose, or, if the level of SMAD7, IL-6, and/or TNFα is unchanged or increased after said administration step compared to the level of SMAD7, IL-6, and/or TNFα before said administration step, then administering to the patient a subsequent dose that is the same as the initial dose or greater than the initial dose or terminating the treatment.

269. A method for treating or managing celiac disease in a patient having celiac disease, wherein the method comprises: (a) establishing a control level of a SMAD7, IL-6, and/or TNFα for the patient; (b) administering to the patient an initial dose of a specific inhibitor of IL-6; (c) analyzing the level of SMAD7, IL-6, and/or TNFα in the patient; and (d) if the level of SMAD7, IL-6, and/or TNFα is lower than the control level, then administering to the patient a subsequent dose that is the same as the initial dose or smaller than the initial dose, or, if the level of SMAD7, IL-6, and/or TNFα is unchanged or increased compared to the control level, then administering to the patient a subsequent dose that is the same as the initial dose or greater than the initial dose or terminating the treatment.

270. The method of claim 268 or 269, wherein, if the level of SMAD7, IL-6, and/or TNFα is at least 10%, at least 20%, at least 30%, at least 40%, at least 50%, or at least 70% decreased after said administration step compared to the level of SMAD7, IL-6, and/or TNFα before said administration step, then administering to the patient a subsequent dose that is the same as the initial dose or smaller than the initial dose.

271. The method of claim 268 or 269, further comprising determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of the celiac disease for a time period of at least 1 week, at least 2 weeks, at least 3 weeks, at least 4 weeks, at least 6 weeks or at least 8 weeks, if the level of SMAD7, IL-6, and/or TNFα after said administering step is decreased at least 10%, at least 20%, at least 30%, at least 40%, at least 50%, at least 60%, or at least 70% compared to the level of SMAD7, IL-6, and/or TNFα before said administration step.

272. The method of claim 265 or claim 267, wherein if the level of SMAD7, IL-6, and/or TNFα is above normal levels of SMAD7, IL-6, and/or TNFα, then administering to the patient a subsequent dose that is greater than the initial dose, or, if the level of SMAD7, IL-6, and/or TNFα is below normal levels of SMAD7, IL-6, and/or TNFα then administering to the patient a subsequent dose that is smaller than the initial dose.

273. The method of claim 267 or 269, wherein, if the subsequent dose is equal to or greater than the maximum tolerated dose (MTD), then terminating the treatment.

274. The method of claim 267 or 269, wherein the level of SMAD7, IL-6, and/or TNFα is analyzed at least 1 day, at least 3 days, at least 5 days, at least 1 week, at least 2 weeks, at least 3 weeks, at least 1 month, at least 2 months, at least 4 months, or at least 6 months after said administration step.

275. The method of claim 267 or 269, wherein the level of SMAD7, IL-6, and/or TNFα is analyzed immediately after said administration step.

276. The method of claim 267 or 269, wherein the level of SMAD7, IL-6, and/or TNFα is analyzed about 15 days or about 28 days after said administration step.

277. The methods of claim 265 or claim 266, wherein the normal levels of SMAD7, IL-6, and/or TNFα are median levels of SMAD7, IL-6, and/or TNFα in a healthy control group.

278. The methods of claim 269, wherein the control level of SMAD7, IL-6, and/or TNFα is a median level of SMAD7, IL-6, and/or TNFα in a healthy control group.

279. The method of claim 277 or 278, wherein the healthy control group and the patient having celiac disease are matched with respect to age, gender, ethnic origin, smoking habits, dietary habits, body-mass index (BMI), and/or exercise habits.

280. The method of claim 265 or claim 266, wherein the SMAD7, IL-6, and/or TNFα is analyzed by measuring the concentration of SMAD7 protein, SMAD7 mRNA, IL-6 protein, IL-6 mRNA, TNFα protein, and/or TNFα mRNA.

281. The method of claim 269, wherein the SMAD7, IL-6, and/or TNFα is analyzed by measuring the concentration of SMAD7 protein, SMAD7 mRNA, IL-6 protein, IL-6 mRNA, TNFα protein, and/or TNFα mRNA.

282. The method of claim 265, 266, or 269, wherein the initial dose is less than 100 mg/day, less than 90 mg/day, less than 80 mg/day, less than 70 mg/day, less than 60 mg/day, less than 50 mg/day, less than 40 mg/day or less than 30 mg/day.

283. The method of claim 265, 266, or 269, wherein the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day.

284. The method of claim 265, 266, or 269, wherein the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.

285. The method of claim 265, 266, or 269, wherein the initial dose is 10 mg/day, 40 mg/day, 80 mg/day, or 160 mg/day.

286. The method of claim 265 or 267, wherein, if SMAD7, IL-6, and/or TNFα levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

287. The method of claim 269, wherein, if SMAD7, IL-6, and/or TNFα levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

288. The method of claim 265 or 267 wherein, if SMAD7, IL-6, and/or TNFα levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.

289. The method of claim 269, wherein, if SMAD7, IL-6, and/or TNFα levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.

290. The method of claim 265, 267, or 269, wherein the initial dose is between about 10 mg/day and 100 mg/day and the subsequent dose is between about 30 mg/day and 200 mg/day.

291. The method of claim 265, 266, or 269, wherein the level of SMAD7, IL-6, and/or TNFα in the patient having celiac disease is determined in a sample obtained from the patient having celiac disease.

292. The method of claim 291, wherein the sample is a blood, serum, plasma, or intestinal tissue sample.

293. The method of claim 265, 266, or 269, wherein the level of SMAD7, IL-6, and/or TNFα is determined by immunochemistry or by nucleotide analysis.

294. The method of claim 293, wherein the level of SMAD7, IL-6, and/or TNFα is determined by an enzyme-linked immunosorbent assay (ELISA).

295. The method of claim 265, 266, or 269, further comprising determining a level of one or more additional analytes in the patient having celiac disease.

296. The method of claim 295, wherein the one or more additional analytes comprise IL-25 and/or IL-15.

297. The method of claim 265, 266, or 269, wherein the specific inhibitor of IL-6 is administered orally to the patient having celiac disease.

298. A method for treating or managing celiac disease in a patient with celiac disease having above normal SMAD7, IL-6, and/or TNFα levels following administration of a dose of a specific inhibitor of IL-6, said method comprising administering to said patient a further dose of said specific inhibitor of IL-6 that is greater than or equal to the prior dose.

299. A method for treating or managing celiac disease in a patient with celiac disease having below normal SMAD7, IL-6, and/or TNFα levels following administration of a dose of specific inhibitor of IL-6, said method comprising administering to said patient a further dose of said specific inhibitor of IL-6 that is less than or equal to the prior dose.

300. A method of treating or managing celiac disease in a patient with celiac disease having above normal SMAD7, IL-6, and/or TNFα levels, said method comprising administering to said patient a dose of a specific inhibitor of IL-6.

301. The method of claim 300, wherein the administering is repeated until a SMAD7, IL-6, and/or TNFα level reaches a normal level.

302. A method of monitoring the treatment or management of celiac disease in a patient with celiac disease, the method comprising analyzing SMAD7, IL-6, and/or TNFα levels in the patient following each specific inhibitor of IL-6 administration, wherein the absence of an decrease in SMAD7, IL-6, and/or TNFα levels indicates that the treatment or management is not effective.

303. The method of claim 302, wherein SMAD7, IL-6, and/or TNFα levels are analyzed one time, two times, three times, four times, about five times, about 10 times, about 15 times, about 20 times, or about 30 times after each administration of specific inhibitor of IL-6.

304. The method of claim 302, wherein the SMAD7, IL-6, and/or TNFα levels are analyzed immediately after, about 1 hour after, about 3 hours after, about 6 hours after, about 12 hours after, about 1 day after, about 3 days after, about 1 week after, about 2 weeks after, and/or about 1 month after specific inhibitor of IL-6 administration.

305. A method of treating or managing celiac disease in a patient with celiac disease having above normal levels of SMAD7, IL-6, and/or TNFα, comprising increasing the amount of a specific inhibitor of IL-6 administered to the patient until SMAD7, IL-6, and/or TNFα levels in the patient decrease.

306. The method of claim 305, wherein SMAD7, IL-6, and/or TNFα decreases to about a normal level of SMAD7, IL-6, and/or TNFα or a below normal level of SMAD7, IL-6, and/or TNFα.

307. A specific inhibitor of IL-6 for use in a method for treating or managing celiac disease in a patient having celiac disease, wherein the method comprises analyzing the level of SMAD7, IL-6, and/or TNFα in the patient to determine appropriate levels of specific inhibitor of IL-6 administration.

308. The specific inhibitor of IL-6 for use of claim 307, wherein the method comprises the steps of: (a) administering to the patient an initial dose of the specific inhibitor of IL-6; (b) analyzing the level of a SMAD7, IL-6, and/or TNFα in the patient; and (c) if the level of SMAD7, IL-6, and/or TNFα is above normal levels of SMAD7, IL-6, and/or TNFα, then administering to the patient a subsequent dose of the specific inhibitor of IL-6 that is greater than or equal to the initial dose, or, if the level of SMAD7, IL-6, and/or TNFα is below normal levels of SMAD7, IL-6, and/or TNFα then administering to the patient a subsequent dose of the specific inhibitor of IL-6 that is equal to or smaller than the initial dose.

309. A specific inhibitor of IL-6 for use in a method for treating or managing celiac disease in a patient having celiac disease, wherein the method comprises (a) analyzing the level of SMAD7, IL-6, and/or TNFα in the patient; and (b) if the level of SMAD7, IL-6, and/or TNFα is above normal levels of SMAD7, IL-6, and/or TNFα, then administering to the patient an initial dose of the specific inhibitor of IL-6.

310. The method of any one of claims 265-306 or the specific inhibitor of IL-6 of any one of claims 307-309, wherein the celiac disease is refractory celiac disease.

311. A method for treating or managing celiac disease in a patient having celiac disease, wherein the method comprises (a) administering to the patient an initial dose of a specific inhibitor of TNFα; (b) analyzing the level of SMAD7, IL-6, and/or TNFα in the patient; and (c) if the level of SMAD7, IL-6, and/or TNFα is above normal levels of SMAD7, IL-6, and/or TNFα, then administering to the patient a subsequent dose that is greater than or equal to the initial dose, or, if the level SMAD7, IL-6, and/or TNFα is below normal levels of SMAD7, IL-6, and/or TNFα, then administering to the patient a subsequent dose that is equal to or smaller than the initial dose.

312. A method for treating or managing celiac disease in a patient having celiac disease, wherein the method comprises (a) analyzing the level of a SMAD7, IL-6, and/or TNFα in the patient; and (b) if the level of SMAD7, IL-6, and/or TNFα is above normal levels of SMAD7, IL-6, and/or TNFα, then administering to the patient an initial dose of a specific inhibitor of TNFα.

313. The method of claim 312, wherein the method further comprises: (c) analyzing the level of SMAD7, IL-6, and/or TNFα in the patient after said administering step; and (d) if the level of SMAD7, IL-6, and/or TNFα is above normal levels of SMAD7, IL-6, and/or TNFα then administering to the patient a subsequent dose that is greater than or equal to the initial dose, or, if the level of SMAD7, IL-6, and/or TNFα is below normal levels of SMAD7, IL-6, and/or TNFα then administering to the patient a subsequent dose that is equal to or smaller than the initial dose.

314. The method of claim 312, wherein the method further comprises: (c) analyzing the level of SMAD7, IL-6, and/or TNFα in the patient after said administering step; and (d) if the level of SMAD7, IL-6, and/or TNFα is decreased after said administration step compared to the level of SMAD7, IL-6, and/or TNFα before said administration step, then administering to the patient a subsequent dose that is the same as the initial dose or smaller than the initial dose, or, if the level of SMAD7, IL-6, and/or TNFα is unchanged or increased after said administration step compared to the level of SMAD7, IL-6, and/or TNFα before said administration step, then administering to the patient a subsequent dose that is the same as the initial dose or greater than the initial dose or terminating the treatment.

315. A method for treating or managing celiac disease in a patient having celiac disease, wherein the method comprises: (a) establishing a control level of a SMAD7, IL-6, and/or TNFα for the patient; (b) administering to the patient an initial dose of a specific inhibitor of TNFα; (c) analyzing the level of SMAD7, IL-6, and/or TNFα in the patient; and (d) if the level of SMAD7, IL-6, and/or TNFα is lower than the control level, then administering to the patient a subsequent dose that is the same as the initial dose or smaller than the initial dose, or, if the level of SMAD7, IL-6, and/or TNFα is unchanged or increased compared to the control level, then administering to the patient a subsequent dose that is the same as the initial dose or greater than the initial dose or terminating the treatment.

316. The method of claim 314 or 315, wherein, if the level of SMAD7, IL-6, and/or TNFα is at least 10%, at least 20%, at least 30%, at least 40%, at least 50%, or at least 70% decreased after said administration step compared to the level of SMAD7, IL-6, and/or TNFα before said administration step, then administering to the patient a subsequent dose that is the same as the initial dose or smaller than the initial dose.

317. The method of claim 314 or 315, further comprising determining that the patient having celiac disease has a greater than 20%, greater than 30%, greater than 40%, greater than 50%, greater than 60%, greater than 70%, greater than 80%, greater than 90% or greater than 100% chance of experiencing clinical amelioration of the celiac disease for a time period of at least 1 week, at least 2 weeks, at least 3 weeks, at least 4 weeks, at least 6 weeks or at least 8 weeks, if the level of SMAD7, IL-6, and/or TNFα after said administering step is decreased at least 10%, at least 20%, at least 30%, at least 40%, at least 50%, at least 60%, or at least 70% compared to the level of SMAD7, IL-6, and/or TNFα before said administration step.

318. The method of claim 311 or claim 313, wherein if the level of SMAD7, IL-6, and/or TNFα is above normal levels of SMAD7, IL-6, and/or TNFα, then administering to the patient a subsequent dose that is greater than the initial dose, or, if the level of SMAD7, IL-6, and/or TNFα is below normal levels of SMAD7, IL-6, and/or TNFα then administering to the patient a subsequent dose that is smaller than the initial dose.

319. The method of claim 313 or 315, wherein, if the subsequent dose is equal to or greater than the maximum tolerated dose (MTD), then terminating the treatment.

320. The method of claim 313 or 315, wherein the level of SMAD7, IL-6, and/or TNFα is analyzed at least 1 day, at least 3 days, at least 5 days, at least 1 week, at least 2 weeks, at least 3 weeks, at least 1 month, at least 2 months, at least 4 months, or at least 6 months after said administration step.

321. The method of claim 313 or 315, wherein the level of SMAD7, IL-6, and/or TNFα is analyzed immediately after said administration step.

322. The method of claim 313 or 315, wherein the level of SMAD7, IL-6, and/or TNFα is analyzed about 15 days or about 28 days after said administration step.

323. The methods of claim 311 or claim 312, wherein the normal levels of SMAD7, IL-6, and/or TNFα are median levels of SMAD7, IL-6, and/or TNFα in a healthy control group.

324. The methods of claim 315, wherein the control level of SMAD7, IL-6, and/or TNFα is a median level of SMAD7, IL-6, and/or TNFα in a healthy control group.

325. The method of claim 323 or 324, wherein the healthy control group and the patient having celiac disease are matched with respect to age, gender, ethnic origin, smoking habits, dietary habits, body-mass index (BMI), and/or exercise habits.

326. The method of claim 311 or claim 312, wherein the SMAD7, IL-6, and/or TNFα is analyzed by measuring the concentration of SMAD7 protein, SMAD7 mRNA, IL-6 protein, IL-6 mRNA, TNFα protein, and/or TNFα mRNA.

327. The method of claim 315, wherein the SMAD7, IL-6, and/or TNFα is analyzed by measuring the concentration of SMAD7 protein, SMAD7 mRNA, IL-6 protein, IL-6 mRNA, TNFα protein, and/or TNFα mRNA.

328. The method of claim 311, 312, or 315, wherein the initial dose is less than 100 mg/day, less than 90 mg/day, less than 80 mg/day, less than 70 mg/day, less than 60 mg/day, less than 50 mg/day, less than 40 mg/day or less than 30 mg/day.

329. The method of claim 311, 312, or 315, wherein the initial dose is at least 10 mg/day, at least 20 mg/day, at least 30 mg/day, at least 40 mg/day, at least 50 mg/day, at least 60 mg/day, at least 70 mg/day, at least 80 mg/day, or at least 90 mg/day.

330. The method of claim 311, 312, or 315, wherein the initial dose is about 10 mg/day, about 20 mg/day, about 30 mg/day, about 40 mg/day, about 50 mg/day, about 60 mg/day, about 70 mg/day, about 80 mg/day, about 90 mg/day, or about 100 mg/day.

331. The method of claim 311, 312, or 315, wherein the initial dose is 10 mg/day, 40 mg/day, 80 mg/day, or 160 mg/day.

332. The method of claim 311 or 313, wherein, if SMAD7, IL-6, and/or TNFα levels are above normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

333. The method of claim 315, wherein, if SMAD7, IL-6, and/or TNFα levels are above a control level, the subsequent dose is at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day greater than the initial dose.

334. The method of claim 311 or 313 wherein, if SMAD7, IL-6, and/or TNFα levels are below normal levels, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.

335. The method of claim 315, wherein, if SMAD7, IL-6, and/or TNFα levels are below a control level, the subsequent dose is at least about 10 mg/day, at least about 20 mg/day, at least about 30 mg/day, at least about 40 mg/day, at least about 50 mg/day, at least about 60 mg/day, at least about 70 mg/day, or at least about 80 mg/day smaller than the initial dose.

336. The method of claim 311, 313, or 315, wherein the initial dose is between about 10 mg/day and 100 mg/day and the subsequent dose is between about 30 mg/day and 200 mg/day.

337. The method of claim 311, 312, or 315, wherein the level of SMAD7, IL-6, and/or TNFα in the patient having celiac disease is determined in a sample obtained from the patient having celiac disease.

338. The method of claim 337, wherein the sample is a blood, serum, plasma, or intestinal tissue sample.

339. The method of claim 311, 312, or 315, wherein the level of SMAD7, IL-6, and/or TNFα is determined by immunochemistry or by nucleotide analysis.

340. The method of claim 339, wherein the level of SMAD7, IL-6, and/or TNFα is determined by an enzyme-linked immunosorbent assay (ELISA).

341. The method of claim 311, 312, or 315, further comprising determining a level of one or more additional analytes in the patient having celiac disease.

342. The method of claim 341, wherein the one or more additional analytes comprise IL-25 and/or IL-15.

343. The method of claim 311, 312, or 315, wherein the specific inhibitor of TNFα is administered orally to the patient having celiac disease.

344. A method for treating or managing celiac disease in a patient with celiac disease having above normal SMAD7, IL-6, and/or TNFα levels following administration of a dose of a specific inhibitor of TNFα, said method comprising administering to said patient a further dose of said specific inhibitor that is greater than or equal to the prior dose.

345. A method for treating or managing celiac disease in a patient with celiac disease having below normal SMAD7, IL-6, and/or TNFα levels following administration of a dose of specific inhibitor of TNFα, said method comprising administering to said patient a further dose of said specific inhibitor that is less than or equal to the prior dose.

346. A method of treating or managing celiac disease in a patient with celiac disease having above normal SMAD7, IL-6, and/or TNFα levels, said method comprising administering to said patient a dose of a specific inhibitor of TNFα.

347. The method of claim 346, wherein the administering is repeated until a SMAD7, IL-6, and/or TNFα level reaches a normal level.

348. A method of monitoring the treatment or management of celiac disease in a patient with celiac disease, the method comprising analyzing SMAD7, IL-6, and/or TNFα levels in the patient following each specific inhibitor of TNFα administration, wherein the absence of an decrease in SMAD7, IL-6, and/or TNFα levels indicates that the treatment or management is not effective.

349. The method of claim 348, wherein SMAD7, IL-6, and/or TNFα levels are analyzed one time, two times, three times, four times, about five times, about 10 times, about 15 times, about 20 times, or about 30 times after each administration of specific inhibitor of TNFα.

350. The method of claim 348, wherein the SMAD7, IL-6, and/or TNFα levels are analyzed immediately after, about 1 hour after, about 3 hours after, about 6 hours after, about 12 hours after, about 1 day after, about 3 days after, about 1 week after, about 2 weeks after, and/or about 1 month after specific inhibitor of TNFα administration.

351. A method of treating or managing celiac disease in a patient with celiac disease having above normal levels of SMAD7, IL-6, and/or TNFα, comprising increasing the amount of a specific inhibitor of TNFα administered to the patient until SMAD7, IL-6, and/or TNFα levels in the patient decrease.

352. The method of claim 351, wherein SMAD7, IL-6, and/or TNFα decreases to about a normal level of SMAD7, IL-6, and/or TNFα or a below normal level of SMAD7, IL-6, and/or TNFα.

353. A specific inhibitor of TNFα for use in a method for treating or managing celiac disease in a patient having celiac disease, wherein the method comprises analyzing the level of SMAD7, IL-6, and/or TNFα in the patient to determine appropriate levels of specific inhibitor of TNFα administration.

354. The specific inhibitor of TNFα for use of claim 353, wherein the method comprises the steps of: (a) administering to the patient an initial dose of the specific inhibitor of TNFα; (b) analyzing the level of a SMAD7, IL-6, and/or TNFα in the patient; and (c) if the level of SMAD7, IL-6, and/or TNFα is above normal levels of SMAD7, IL-6, and/or TNFα, then administering to the patient a subsequent dose of the specific inhibitor of TNFα that is greater than or equal to the initial dose, or, if the level of SMAD7, IL-6, and/or TNFα is below normal levels of SMAD7, IL-6, and/or TNFα then administering to the patient a subsequent dose of the specific inhibitor of TNFα that is equal to or smaller than the initial dose.

355. A specific inhibitor of TNFα for use in a method for treating or managing celiac disease in a patient having celiac disease, wherein the method comprises (a) analyzing the level of SMAD7, IL-6, and/or TNFα in the patient; and (b) if the level of SMAD7, IL-6, and/or TNFα is above normal levels of SMAD7, IL-6, and/or TNFα, then administering to the patient an initial dose of the specific inhibitor of TNFα.

356. The method of any one of claims 311-352 or the specific inhibitor of TNFα of any one of claims 353-355, wherein the celiac disease is refractory celiac disease.

357. A SMAD7 antisense oligonucleotide for use as a medicament.

358. A SMAD7 antisense oligonucleotide for use in treating celiac disease.

359. The SMAD7 antisense oligonucleotide for use as claimed in claim 358, wherein treatment of celiac disease is by a method as claimed in any one of claims 26 to 71 or 214 to 260.

360. The SMAD7 antisense oligonucleotide for use as claimed in any one of claim 73-75 or 261-263, wherein the SMAD7 antisense oligonucleotide comprises the nucleotide sequence of SEQ ID NO: 2 (5′-GTCGCCCCTTCTCCCCGCAGC-3′).

361. The SMAD7 antisense oligonucleotide for use as claimed in any one of claim 73-75 or 261-263, wherein the antisense oligonucleotide is a SMAD7 phosphorothioate antisense oligonucleotide comprising the following sequence: 5′-GTXGCCCCTTCTCCCXGCAG-3′ (SEQ ID NO: 3) wherein X is a nucleotide comprising 5-methyl-2′-deoxycytidine and wherein the internucleotide linkages are phosphorothioate linkages.

362. The SMAD7 antisense oligonucleotide for use as claimed in any one of claim 73-75 or 261-263, wherein the antisense oligonucleotide is a SMAD7 phosphorothioate antisense oligonucleotide comprising the following sequence: 5′-GTXGCCCCTTCTCCCXGCAGC-3′ (SEQ ID NO: 4) wherein X is a nucleotide comprising 5-methyl-2′-deoxycytidine and wherein the internucleotide linkages are phosphorothioate linkages.

363. The method of any one of claim 214-258, 264-297, 302-304, 310, 311-343, 348-350, or 356, wherein the level of SMAD7 is analyzed by measuring the concentration of SMAD7 protein.

364. The method of any one of claim 259, 260, 264, 298-301, 305, 306, 310, 344-347, 351, 352, or 356, further comprising analyzing the level of SMAD7 by measuring the concentration of SMAD7 protein.

365. The SMAD7 antisense oligonucleotide for use of claims 261-264, wherein the method comprises analyzing the level of SMAD7 by measuring the concentration of SMAD7 protein.

366. The specific inhibitor of IL-6 for use of claims 307-310, wherein the method comprises analyzing the level of SMAD7 by measuring the concentration of SMAD7 protein.

367. The specific inhibitor of TNFα for use of claims 353-356, wherein the method comprises analyzing the level of SMAD7 by measuring the concentration of SMAD7 protein.
